(19) United States
(12) Patent Application Publication
(10) Pub. No.: US 2025/0304655 A1
(43) Pub. Date: Oct. 2, 2025

(54) Expression System for Producing a Recombinant Haptoglobin (Hp) Beta Chain

(71) Applicants: CSL Behring AG, Bern (CH); Universitaet Zuerich, Zuerich (CH)

(72) Inventors: Rebecca Butcher, Williamstown, Victoria (AU); Catherine Owczarek, Northcote, Victoria (AU); Thomas Gentinetta, Bern (CH); Dominik Schaer, Zuerich (CH); Michael Hugelshofer, Mannheim (DE)

(73) Assignees: CSL Behring AG, Bern (CH); Universitaet Zuerich, Zuerich (CH)

(21) Appl. No.: 18/557,126

(22) PCT Filed: May 6, 2022
(86) PCT No.: PCT/EP2022/062257

§ 371 Date: Oct. 25, 2023

(30) Foreign Application Priority Data
May 7, 2021 (AU) 2021901366

(51) Int. Cl. C07K 14/805; A61K 38/42; C12N 15/85

(52) U.S. Cl. CPC C07K 14/805; A61K 38/42; C12N 15/85; C07K 2319/50; C12N 2510/02

(57) ABSTRACT

The present invention relates to an expression system for producing a recombinant haptoglobin (Hp) beta chain, or a haemoglobin-binding fragment thereof, recombinant Hp molecules and uses thereof for treating and/or preventing a condition associated with cell-free haemoglobin (Hb).

### CROSS-REFERENCE TO RELATED APPLICATIONS

This application is a national phase entry pursuant to 35 U.S.C. § 371 of International Application No. PCT/EP2022/062257, filed May 6, 2022, which claims priority to Australian Application No. 2021901366, filed May 7, 2021, the contents of which are incorporated by reference in their entireties.

### SEQUENCE LISTING

The instant application contains a Sequence Listing which has been submitted electronically in text format and is hereby incorporated by reference in its entirety. Said text copy, created on Apr. 16, 2024, is named “2024-04-16-01113-0027-00US-Corrected-ST25.txt” and is 92,280 bytes in size.

### TECHNICAL FIELD

The present invention relates generally to an expression system for producing a recombinant haptoglobin (Hp) beta chain, or a haemoglobin-binding fragment thereof, recombinant Hp molecules and uses thereof for treating and/or preventing conditions associated with aberrant levels of cell-free hemoglobin (Hb).

### BACKGROUND

Erythrolyis is characterised by the rupture of red blood cells (erythocytes), causing the release of hemoglobin (Hb) into blood plasma and is a hall-mark of anaemic disorders associated with red blood cell abnormalities, such as enzyme defects, haemoglobinopathies (e.g., thalassemias), hereditary spherocytosis, paroxysmal nocturnal haemoglobinuria and spur cell anaemia, as well as extrinsic factors such as splenomegaly, autoimmune disorders (e.g., Haemolytic disease of the newborn), genetic disorders (e.g., Sickle-cell disease or G6PD deficiency), microangiopathic haemolysis, Gram-positive bacterial infection (e.g., Streptococcus, Enterococcus and Staphylococcus), parasite infection (e.g., Plasmodium), toxins, trauma (e.g., burns), haemorrhagic stroke, sepsis, atherosclerosis, blood transfusions (in particular massive blood transfusions) and in patients using an extracorporeal cardio-pulmonary support (see Hoppe et al. (1998, Curr Opin Pediatr, 10(1):49-52); Roumenina (2016; Trends in Molecular Medicine, 22(3):200-213); Merle (2019, PNAS 116 (13):6280-6285); Larsen (2010, Science Translational Medicine: 2(51):51ra71); and Balla G (2019, Int J Mol Sci; 20(15):3675).

The adverse effects seen in patients with conditions associated with haemolysis are largely attributed to the release of iron and iron-containing compounds from red blood cells, such as Hb and heme. Under physiological conditions, cell-free haemoglobin is typically bound by soluble proteins such as haptoglobin (Hp) (see C. B. F. Andersen et al., 2012, Nature, 489(7416):456-459). and transported to macrophages and hepatocytes. However, in circumstances where the incidence of haemolysis is accelerated and/or becomes pathological in nature, the buffering capacity of Hp is overwhelmed. As a result, Hb is quickly oxidised to ferri-haemoglobin, which in turn releases free heme (comprising protoporphyrin IX and iron; see Schaer et al. (2014; frontiers in PHYSIOLOGY, 5:1-13)). Whilst heme plays a critical role in several biological processes (e.g., as part of essential proteins such as haemoglobin and myoglobin), free heme is highly toxic. For instance, free heme is a source of redox-active iron, which in turn produces highly toxic reactive oxygen species (ROS) that damages lipid membranes (see Deuel et al. (2015; Free Radical Biology and Medicine, 89:931-943), proteins and nucleic acids. Heme toxicity is further exacerbated by its ability to intercalate into lipid membranes, where it causes oxidation of membrane components and promotes cell lysis and death (see Jeney et al. (2002; Blood, 100(3):879-87).

The evolutionary pressure of continuous low-level extracellular Hb/heme exposure has led to compensatory mechanisms that control the adverse effects of free Hb/heme under physiological steady-state conditions and during mild haemolysis. These systems include the release of a group of plasma proteins that bind Hb or heme, including the Hb scavenger Hp and heme scavenger proteins, such as hemopexin (Hpx) and α1-microglobulin (see Schaer et al. 2013; Blood, 121(8):1276-84).

As noted above, plasma Hp acts a scavenger for cell-free Hb, binding to cell-free Hb to form a neutralised Hb:Hp complex (see Shim et al. 1965, Nature, 207:1264-1267). However, when the amount of Hb exceeds the scavenging capacity of plasma Hp, local accumulation of Hb, particularly in vascular and renal tissues, results in oxidative stresses that may lead to adverse secondary outcomes for patients. The protection provided by Hp attenuates at least two toxicological consequences of Hb. First, the large molecular size of the Hb:Hp complex prevents extravasation of cell free Hb. This mechanism protects renal function and preserves vascular nitric oxide (NO) homeostasis by limiting access of free Hb into the vascular wall (see Azarov et al., 2008, Nitric Oxide; 18(4):296-302). Secondly, Hb:Hp complex formation stabilizes the structure of the Hb molecule in a way that limits transfer of heme from its globin chains to proteins and reactive lipids (see Schaer et al. (2014; Frontiers in Physiology, 5:1-13). These mechanisms are largely responsible for the anti-oxidative function of Hp following haemolysis.

While endogenous Hp could principally provide significant protection against cell free Hb toxicity, it is rapidly consumed and depleted during more pronounced acute or prolonged haemolysis (see Boretti et al., 2014; Frontiers in Physiology, 5:385). Replacement of Hp has therefore being considered as a therapeutic modality demonstrating preclinical proof-of-concept in vitro and in animal models of haemolysis. For the most part, preclinical studies have evaluated the therapeutic potential of Hp purified from pooled human plasma fractions. However, this approach has several limitations that are relevant to clinical practice, such as (1) the mixture of different Hp phenotypes (1-1, 2-1 and 2-2) may trigger neutralizing antibody responses in some patients during prolonged replacement therapy, (2) differing phenotypes may afford differing efficacy and (3) phenotypic forms may demonstrate different pharmacokinetics. Considering the potential limitations of plasma derived Hp, recombinant protein production may therefore offer a relevant therapeutic strategy that avoids or otherwise alleviates at least some of the aforementioned limitations of plasma-derived Hp. Additionally, recombinant protein-production strategies may generate therapeutics with enhanced functionality, bioavailability and pharmacokinetics. However, recent attempts to produce recombinant Hp by expressing the precursor molecule (proHp) have noted reduced binding to Hb (see Heinderyckx et al., 1988-1989, Mol Biol Rep; 13(4):225-32). Hence, there remains an ongoing need for alternative or improved therapies to treat and/or prevent conditions associated with cell-free Hb in which the Hb-scavenging properties of Hp would be beneficial.

### SUMMARY

The present invention is predicated, at least in part, on the inventors' surprising finding that a functional haptoglobin beta chain, or a haemoglobin-binding fragment thereof, can be produced in a mammalian expression system from an N-terminal truncated pro-haptoglobin (proHp). Moreover, the N-terminal truncated proHp may be advantageously modified to carry a functional moiety, such as Hpx, Fc or albumin, thereby producing a construct with improved therapeutic properties.

Thus, in one aspect disclosed herein, there is provided an expression system for producing a recombinant haptoglobin beta chain, or a haemoglobin-binding fragment thereof, in a mammalian cell, the expression system comprising:
(a) a first nucleic acid sequence encoding an N-terminal truncated pro-haptoglobin (proHp), wherein the N-terminal truncated proHp comprises (i) at least 14 contiguous C-terminal amino acid residues of a haptoglobin alpha chain and (ii) a haptoglobin beta chain, or a haemoglobin-binding fragment thereof, and wherein the N-terminal truncated proHp comprises an internal enzymatic cleavage site between the at least 14 contiguous C-terminal amino acid residues of a haptoglobin alpha chain and the haptoglobin beta chain, or haemoglobin-binding fragment thereof, and
(b) a second nucleic acid sequence encoding an enzyme capable of cleaving the N-terminal truncated proHp at the enzymatic cleavage site;
   wherein, upon introduction of the first nucleic acid sequence and the second nucleic acid sequence into a mammalian cell, and subsequent expression of the N-terminal truncated proHp and the enzyme in the cell, the enzyme is capable of cleaving the N-terminal truncated proHp at the internal enzymatic cleavage site, thereby releasing the haptoglobin beta chain, or haemoglobin-binding fragment thereof, from the N-terminal truncated proHp.

In another aspect disclosed herein, there is provided an expression vector for producing a recombinant haptoglobin beta chain, or a haemoglobin-binding fragment thereof, in a mammalian cell, wherein the vector comprises:
(a) the first nucleic acid sequence as herein described; and
(b) the second nucleic acid sequence as herein described.

The present disclosure also extends to a mammalian cell comprising the expression system or the expression vector as herein described.

In another aspect disclosed herein, there is provided a method of producing a recombinant haptoglobin beta chain, or a haemoglobin-binding fragment thereof, the method comprising:
(a) introducing into a mammalian cell the expression system as herein described to produce a modified mammalian cell;
(b) culturing the modified mammalian cell produced in step (a) under conditions and for a period of time sufficient to allow production of the recombinant haptoglobin beta chain, or the haemoglobin-binding fragment thereof; and
(c) collecting the recombinant haptoglobin beta chain, or the haemoglobin-binding fragment thereof produced in step (b).

In another aspect disclosed herein, there is provided a recombinant haptoglobin beta chain, or a haemoglobin-binding fragment thereof, produced by the methods as herein described.

In another aspect disclosed herein, there is provided a recombinant haemoglobin-binding molecule comprising (i) a haptoglobin beta chain, or a haemoglobin-binding fragment thereof, and (ii) an N-terminal truncated haptoglobin alpha chain, wherein the N-terminal truncated haptoglobin alpha chain comprises at least 14 contiguous C-terminal amino acid residues of the haptoglobin alpha chain, wherein the at least 14 contiguous C-terminal amino acid residues of the haptoglobin alpha chain is non-contiguous to the haptoglobin beta chain, or the haemoglobin-binding fragment thereof, and wherein the N-terminal truncated haptoglobin alpha chain is attached to the haptoglobin beta chain, or the haemoglobin-binding fragment thereof.

The present disclosure also extends to a pharmaceutical composition comprising a therapeutically effective amount of the recombinant haemoglobin-binding molecule, as herein described, or the recombinant haptoglobin beta chain, or a haemoglobin-binding fragment thereof, as herein described, and a pharmaceutically acceptable carrier.

In another aspect disclosed herein, there is provided a method of treating or preventing a condition associated with cell-free haemoglobin (Hb) in a subject, the method comprising administering to a subject in need thereof a therapeutically effective amount of the recombinant haemoglobin-binding molecule, as herein described, or the recombinant haptoglobin beta chain, or haemoglobin-binding fragment thereof, as herein described, and for a period of time sufficient to allow the haptoglobin beta chain, or haemoglobin-binding fragment thereof, to form a complex with, and thereby neutralise, the cell-free Hb. In an embodiment, the condition is associated with erythrolysis.

Also disclosed herein is a pharmaceutical composition for use in treating or preventing a condition associated with cell-free haemoglobin (Hb) in a subject, the composition comprising a therapeutically effective amount of the recombinant haemoglobin-binding molecule, as herein described, or the recombinant haptoglobin beta chain, or a haemoglobin-binding fragment thereof, as herein described, and a pharmaceutically acceptable carrier.

In another aspect disclosed herein, there is provided use of a therapeutically effective amount of the recombinant haemoglobin-binding molecule, as herein described, or the recombinant haptoglobin beta chain, or haemoglobin-binding fragment thereof, as herein described, in the manufacture of a medicament for treating or preventing a condition associated with cell-free haemoglobin (Hb) in a subject.

The present disclosure also extends to a therapeutically effective amount of the recombinant haemoglobin-binding molecule, as herein described, or the recombinant haptoglobin beta chain, or haemoglobin-binding fragment thereof, as herein described, for use in the treatment or prevention of a condition associated with cell-free haemoglobin (Hb) in a subject.

All references, including any patents or patent application, cited in this specification are hereby incorporated by reference to enable full understanding of the invention. Nevertheless, the reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

### BRIEF DESCRIPTION OF THE DRAWINGS

Embodiments of the invention will now be described with reference to the following Figures, which are intended to be exemplary only, and in which:

FIG. 1 (FIGS. 1A-1B) shows the structure of an illustrative example of human haptoglobin; (FIG. 1A) a schematic representation of human Hp1 (SEQ ID NO: 20) and Hp2 (SEQ ID NO: 21). The amino acid sequence that is common to the a-chain of Hp1 and Hp2 is shown and highlighted in green (SEQ ID NOs:14 and 17). The amino acid sequence shaded in blue within the α-chain of Hp2 (second [middle]line of Hp2 sequence) determines the distinct molecular phenotypes. The asterisks specify the cysteine residues required for disulphide bond formation. The arrow indicates the C1rLP cleavage site. (FIG. 1B) the protein quaternary structure of the Hp 1-1 homo-dimer with one inter-α-chain disulphide bond and three variants of Hp 2-2 cyclic homo-multimers with two inter-a-disulphide bonds.

FIG. 2 depicts spectral deconvolution in order to follow the transition of heme-albumin to heme-Hpx. Serial UV-VIS spectra were recorded over time (3h at 37° C.) with reaction mixtures containing 12.5 μM heme-albumin and human Hpx. The first spectrum (t0) is highlighted in orange (light) and the last spectrum (t 3h) in blue (dark).

FIG. 3 (FIGS. 3A-3D) shows (FIG. 3A) the amino acid sequence of human prohaptoglobin 2FS (SEQ ID NO: 1). The signal peptide (amino acid residues 1-18 of SEQ ID NO: 1; MSALGAVIALLLWGQLFA) is highlighted in yellow. The C1rLP cleavage site after Arg161 (R) is indicated by an arrow. The alpha (α) chain is highlighted in blue (amino acid residues 19-161) and the beta (β) chain is highlighted in light green (amino acid residues 162-406). The cysteine residues that form the inter- and intra-chain disulphide bonds are at amino acid positions 33, 52, 86, 92, 149, 266, 309, 340, 351 and 381. The CD163 binding site is identified by amino acid residues 318, 320, 322 and 323. The amino acid residues of the variants described herein are numbered with +1 as the initiator methionine and are based on the amino acid sequence of Hp2FS. Amino acids Asp70, Lys71, Asn129 and Glu130 are highlighted in that regard. (FIG. 3B) Schematic representation of the processing of the pro-haptoglobin 2FS polypeptide chain into an alpha and beta chain. The location of the inter- and intra-chain disulphide bonds (S—S) are indicated. (FIG. 3C) Coomassie stained reducing SDS-PAGE of rHp1S and rHp2FS produced in transfected FS293F cells. Hp α-chain appears at 12 kDa or 19 kDa and Hp β-chain appears at 47 kDa. Additionally, uncleaved proHp1 and proHp2 appear at their expected size of 53 kDa or 57 kDa. With co-expression of C1r-LP (+) all proHp is efficiently cleaved into its subunits. C1r-LP appears at 68 kDa. (FIG. 3D) Anti-8His (SEQ ID NO: 18) Western blot showing uncleaved proHp in the absence of C1r-LP and smaller Hp β-chain as well as the His-tagged protease in the presence of C1r-LP co-expression.

FIG. 4 (FIGS. 4A-4D) shows haptoglobin beta fragment expression in Expi293F cells and purification. (FIG. 4A) Schematic diagram depicting recombinant beta fragment constructs each with C-terminal 8×His tags (SEQ ID NO: 18). The amino acids and the location of the intra-chain disulphide bonds (S—S) are indicated. (FIG. 4B) Schematic diagram depicting recombinant beta fragment construct with additional 14 amino acids with a C-terminal 8×His tag (SEQ ID NO: 18). The processing of the pre-protein by C1r-LP, amino acids and the location of the inter-chain and intra-chain disulphide bonds (S—S) are indicated. (FIG. 4C) (Left panel) Coomassie stained reducing SDS-PAGE of recombinant Hp beta fragment constructs produced in transiently transfected Expi293F cells. The construct encoding the N-terminally extended beta fragment HuHaptoglobin2FS(148-406)-8His (SEQ ID NO: 18) was co-transfected with a construct encoding Hu-C1r-LP-FLAG to ensure cleavage of the nascent polypeptide. (Middle panel) Anti-His western blot of reducing SDS-PAGE of recombinant Hp beta fragment constructs produced in transiently transfected Expi293F cells. (Right panel) Anti-Hp Western blot of reducing SDS-PAGE of recombinant Hp beta fragment constructs produced in transiently transfected Expi293F cells. (FIG. 4D) (Left panel) Analytical SEC chromatogram of nickel-affinity purified, N-terminally extended HuHaptoglobin2FS(148-406)-8His (SEQ ID NO: 18) performed on an Agilent 1260 Infinity HPLC with a Superdex 200 Increase 5/150 column and MT-PBS mobile phase. (Right panel) A 4-12%, Bis-Tris SDS-PAGE gel showing the characteristic glycoform, doublet band of haptoglobin running above its backbone Mw of 29.9 kDa.

FIG. 5 (FIGS. 5A-5B) provides schematics showing haptoglobin beta fragment fusion protein molecule design. (FIG. 5A) Schematic diagram depicting recombinant Hp beta fragment constructs (amino acids 162-406) with either N-terminal or C-terminal fusion partners. The amino acids and the location of the intra-chain disulphide bonds (S—S) are indicated. (FIG. 5B) Schematic diagram depicting recombinant Hp beta fragment constructs with additional N-terminal 14 amino acids (amino acids 148-406) with either N-terminal or C-terminal fusion partners. The processing of the pre-protein by C1r-LP. Amino acids and the location of the inter-chain and intra-chain disulphide bonds (S—S) are indicated.

FIGS. 6 (FIGS. 6A-6C) shows Hu-Hemopexin-Hu-Haptoglobin beta fusion protein expression in Expi293F cells and purification. (FIG. 6A) Schematic diagram depicting recombinant beta fragment constructs containing (i) human hemopexin (Hpx, amino acids 1-462) at the N-terminus, followed by a Gly-Ser linker and then fused to: the human Hp beta fragment encoding amino acids 162-406); (ii) the human Hp beta fragment encoding amino acids 162-406, where the unpaired cysteine at amino acid 266 was mutated to alanine; (iii) the human Hp beta fragment encoding amino acids 148-406 that retains the C1r-LP cleavage site and the cysteine required for the intra-chain disulphide bond. The amino acids and the location of the inter-chain disulphide bonds (S—S) are indicated. (FIG. 6B) (Left panel) Coomassie stained reducing SDS-PAGE of recombinant Hpx-Hp beta fragment constructs produced in transiently transfected Expi293F cells. The construct encoding the N-terminally extended beta fragment HuHaptoglobin2FS(148-406) was co-transfected with a construct encoding Hu-C1r-LP-FLAG to ensure cleavage of the nascent polypeptide. (Middle panel) Anti-His western blot of reducing SDS-PAGE of recombinant Hpx-Hp beta fragment constructs produced in transiently transfected Expi293F cells. (Right panel) Anti-Hp Western blot of reducing SDS-PAGE of recombinant Hp beta fragment constructs produced in transiently transfected Expi293F cells. (FIG. 6C) Analysis of aggregate content and protein processing by SEC and SDS-PAGE. (i) Preparative SEC chromatogram of nickel affinity purified HuHemopexin-HuHaptoglobin2FS(162-406)-8His (SEQ ID NO: 18) performed on an ÄKTAxpress system with a Superdex 200 16/600 column and MT-PBS mobile phase. The position of the arrow shows the peak containing fusion protein of the expected size. (ii) Analytical SEC chromatogram of nickel-affinity purified, N-terminally extended HuHemopexin-HuHaptoglobin2FS(148-406)-8His (SEQ ID NO: 18)/Hu-C1r-LP-FLAG as performed on an Agilent 1260 Infinity HPLC system with a Superdex 200 Increase 5/150 column and MT-PBS mobile phase. The position of the arrow shows the peak containing fusion protein of the expected size. (iii) Reducing and non-reducing SDS-PAGE gels (4-12%, Bis-Tris) showing purity and correct processing of nickel-affinity purified HuHemopexin-HuHaptoglobin2FS(148-406)-8His (SEQ ID NO: 18).

FIG. 7 (FIGS. 7A-7C) shows HSA-Hu-Haptoglobin beta fusion protein expression in Expi293F cells and purification. (FIG. 7A) Schematic diagram depicting recombinant beta fragment constructs containing human serum albumin (HSA) at the N-terminus and fused to (i) a Gly-Ser linker followed by the human Hp beta fragment encoding amino acids 162-406); (ii) a Gly-Ser linker followed by the human Hp beta fragment encoding amino acids 162-406 where the unpaired cysteine at amino acid 266 was mutated to alanine; (iii) the human Hp beta fragment encoding amino acids 148-406 that retains the C1r-LP cleavage site and the cysteine required for the intra-chain disulphide bond. The amino acids and the location of the inter-chain disulphide bonds (S—S) are indicated. (FIG. 7B) (Left panel) Coomassie stained reducing SDS-PAGE of recombinant HSA-Hp beta fragment constructs produced in transiently transfected Expi293F cells. The construct encoding the N-terminally extended beta fragment HuHaptoglobin2FS(148-406) was co-transfected with a construct encoding Hu-C1r-LP-FLAG to ensure cleavage of the nascent polypeptide. (Middle panel) Anti-HSA western blot of reducing SDS-PAGE of recombinant HSA-Hp beta fragment constructs produced in transiently transfected Expi293F cells.(Right panel) Anti-Hp Western blot of reducing SDS-PAGE of recombinant Hp beta fragment constructs produced in transiently transfected Expi293F cells. (FIG. 7C) Analysis of aggregate content and protein processing by SEC and SDS-PAGE. (i) Preparative SEC chromatogram of HSA-affinity purified HSA-GS13-HuHaptoglobin(162-406) performed on an ÄKTAxpress system with a Superdex 200 16/600 column and MT-PBS mobile phase. The position of the arrow shows the peak containing fusion protein of the expected size. (ii) Analytical SEC chromatogram of HSA-affinity purified, N-terminally extended HSA-HuHaptoglobin2FS(148-406/Hu-C1r-LP-FLAG performed on an Agilent 1260 Infinity HPLC system with a Superdex 200 Increase 5/150 column and MT-PBS mobile phase. The position of the arrow shows the peak containing fusion protein of the expected size. (iii) Reducing and non-reducing SDS-PAGE gels (4-12%, Bis-Tris) showing purity and correct processing of HSA-affinity purified HSA-HuHaptoglobin2FS(148-406).

FIG. 8 (FIGS. 8A-8C) shows Fc-Hu-Haptoglobin beta fusion protein expression in Expi293F cells and purification. (FIG. 8A). Schematic diagram depicting recombinant beta fragment constructs containing i) human IgG1 Fc fused to the N-terminus of the human Hp beta fragment encoding amino acids 162-406); (ii) mouse IgG2a followed by the human Hp beta fragment encoding amino acids 148-406 that retains the C1r-LP cleavage site and the cysteine required for the intra-chain disulphide bond. The amino acids and the location of the inter-chain disulphide bonds (S—S) are indicated. (FIG. 8B) (Left panel) Coomassie stained reducing SDS-PAGE of recombinant Fc-Hp beta fragment constructs produced in transiently transfected Expi293F cells. The construct encoding the N-terminally extended beta fragment HuHaptoglobin2FS(148-406) was co-transfected with a construct encoding Hu-C1r-LP-FLAG to ensure cleavage of the nascent polypeptide. (Middle panel) Anti-Fc western blot of reducing SDS-PAGE of recombinant Fc-Hp beta fragment constructs produced in transiently transfected Expi293F cells. (Right panel) Anti-Hp Western blot of reducing SDS-PAGE of recombinant Hp beta fragment constructs produced in transiently transfected Expi293F cells. (FIG. 8C) Analysis of aggregate content and protein processing by SEC and SDS-PAGE. (i) Analytical SEC chromatogram of Protein A affinity purified, N-terminally extended muIgG2aFc-HuHaptoglobin2FS(148-406)/Hu-C1r-LP-FLAG as performed on an Agilent 1260 Infinity system with a Superdex 200 Increase 5/150 column and MT-PBS mobile phase. The position of the arrow shows the peak containing fusion protein of the expected size. (ii) Reducing and non-reducing SDS-PAGE gels (4-12%, Bis-Tris) showing purity and correct processing of Protein A affinity purified muIgG2aFc-HuHaptoglobin2FS(148-406).

FIG. 9 (FIGS. 9A-9C) shows Hemopexin-MSA-Hu-Haptoglobin beta fusion protein expression in Expi293F cells and purification. (FIG. 9A). Schematic diagram depicting recombinant Hp beta fragment constructs containing human hemopexin (Hpx, amino acids 1-462) at the N-terminus, followed by mouse serum albumin (msa) and then fused to i) the human Hp beta fragment encoding amino acids 162-406 ii) human Hp beta fragment encoding amino acids 148-406 that retains the C1r-LP cleavage site and the cysteine required for the intra-chain disulphide bond. The amino acids and the location of the inter-chain disulphide bonds (S—S) are indicated. (FIG. 9B). (Left panel) Coomassie stained reducing SDS-PAGE of recombinant Hpx-msa-Hp beta fragment constructs produced in transiently transfected Expi293F cells. The construct encoding the N-terminally extended beta fragment HuHaptoglobin2FS(148-406) was co-transfected with a construct encoding Hu-C1r-LP-FLAG to ensure cleavage of the nascent polypeptide. (Middle panel) Anti-MSA Western blot of reducing SDS-PAGE of recombinant Fc-Hp beta fragment constructs produced in transiently transfected Expi293F cells. (Right panel) Anti-Hp Western blot of reducing SDS-PAGE of recombinant Hp beta fragment constructs produced in transiently transfected Expi293F cells. (FIG. 9C) Analysis of aggregate content and protein processing by SEC and SDS-PAGE. (i) Preparative SEC chromatogram of CaptureSelect HSA affinity purified HuHemopexin-HSA-HuHaptoglobin2FS(162-406) performed on an ÄKTAxpress system with a Superdex 200 16/600 column and MT-PBS mobile phase. The position of the arrow shows the peak containing fusion protein of the expected size. (ii) Analytical SEC chromatogram of Mimetic Blue affinity purified, N-terminally extended HuHemopexin-MSA-HuHaptoglobin2FS(148-406)/Hu-C1r-LP-FLAG as performed on an Agilent 1260 Infinity HPLC system with a Superdex 200 Increase 5/150 column and MT-PBS mobile phase. The position of the arrow shows the peak containing fusion protein of the expected size. (iii) Reducing and non-reducing SDS-PAGE gels (4-12%, Bis-Tris) showing purity and correct processing of Mimetic Blue affinity purified HuHemopexin-MSA-HuHaptoglobin2FS(148-406).

FIG. 10 (FIGS. 10A-10C) shows HuHemopexin-mIgG2aFc-HuHaptoglobin2FS(148-406) fusion protein expression in Expi293F cells and purification. (FIG. 10A). Schematic diagram depicting a recombinant Hp beta fragment construct containing human human hemopexin (Hpx, amino acids 1-462) at the N-terminus, followed by a Gly-Ser linker, mouse IgG2aFc and then fused to the human Hp beta fragment encoding amino acids 148-406 that retains the C1r-LP cleavage site and the cysteine required for the intra-chain disulphide bond. The amino acids and the location of the inter-chain disulphide bonds (S—S) are indicated. (FIG. 10B). (Left panel) Coomassie stained reducing SDS-PAGE of recombinant HuHemopexin-mIgG2aFc-HuHaptoglobin2FS(148-406) construct produced in transiently transfected Expi293F cells. The construct was co-transfected with a construct encoding Hu-C1r-LP-FLAG to ensure cleavage of the nascent polypeptide. (Middle panel) Anti-Fc western blot of reducing SDS-PAGE of recombinant Fc-Hp beta fragment constructs produced in transiently transfected Expi293F cells. (Right panel) Anti-Hp Western blot of reducing SDS-PAGE of recombinant Hp beta fragment constructs produced in transiently transfected Expi293F cells. (FIG. 10C) Analysis of aggregate content and protein processing by SEC and SDS-PAGE. (i) Analytical SEC chromatogram of Protein A affinity purified, N-terminally extended HuHemopexin-mIgG2aFc-HuHaptoglobin2FS(148-406)/Hu-C1r-LP-FLAG as performed on an Agilent 1260 Infinity system with a Superdex 200 Increase 5/150 column and MT-PBS mobile phase. (ii) Reducing and non-reducing SDS-PAGE gels (4-12%, Bis-Tris) showing purity and correct processing of Protein A affinity purified HuHemopexin-mIgG2aFc-HuHaptoglobin2FS(148-406).

FIG. 11 (FIGS. 11A-11B) shows qualitative Hb binding data based on size exclusion HPLC chromatograms. The blue lines represent the signals (405 nm) of the Hb+rHp mixtures (at equimolar concentrations) (FIG. 11A). HuHaptoglobin(148-406)-8His (SEQ ID NO: 18), HSA-HuHaptoglobin(148-406)-8His (SEQ ID NO: 18) and muIgG2aFc-HuHaptoglobin(148-406)-8His (SEQ ID NO: 18). (FIG. 11B) HuHemopexin-HuHaptoglobin(148-406)-8His (SEQ ID NO: 18), HuHemopexin-msa-HuHaptoglobin2FS(148-406)-8His (SEQ ID NO: 18) and HuHemopexin-mIgG2aFc-HuHaptoglobin2FS(148-406)-His. Hemopexin was used as negative control. The red line shows the signal of Hb alone recorded at 405 nm. All size exclusion HPLC traces are scaled identically to fit to the red Hb peak chromatogram.

FIG. 12 (FIGS. 12A-12C) shows representative sensograms for each Hp variant analysed regarding its ability to bind hemoglobin. Individual Hp variants were immobilized on the biosensor surface. After baseline recording seven concentrations of hemoglobin were tested (15, 7.5, 3.75, 1.88, 0.94, 0.47 and 0.32 nM). After reference subtraction (assay buffer) the data was processed and globally fitted using a 1:1 binding model. The fitting accuracy was described by Chi2 and R2. Hb is shown in grey curves, fitted curve as solid red line. (FIG. 12A) HuHaptoglobin 1-1. (FIG. 12B) HuHaptoglobin2FS(148-406)-8His (SEQ ID NO: 18). (FIG. 12C) HuHemopexin-HuHaptoglobin2FS(148-406)-8His (SEQ ID NO: 18).

FIG. 13 shows the heme binding capacity of different haptoglobin variants containing a hemopexin domain. Heme release from heme-albumin was measured in presence of different Hp variants as indicated using an by recording serial UV-VIS spectra over time (5 h at 37° C.) with reaction mixtures containing 12.5 μM Hb(Fe3+) and 5 μM Hp protein (except for HuHemopexin-msa-HuHaptoglobn2FS(148-406), 4 μM was used). Heme-albumin (blue curve) shows concentration of heme bound to Hb at any given timepoint. Hemopexin:heme (red curve) shows the concentration of heme transferred from heme-albumin at any given timepoint.

FIGS. 14 (FIGS. 14A-14C) shows representative sensograms for each Hp variant analysed regarding its ability to bind to the scavenger receptor CD163. The human CD163 receptor was immobilized on the biosensor surface. After baseline recording six concentrations of complexes were tested. After reference subtraction (assay buffer), data was processed and globally fitted using a 1:1 binding model. The fitting accuracy was described by Chi2 and R2. Hb is shown in grey curves, fitted curve as solid red line. In addition the KD was determined by steady state analysis for the two recombinant variants. (FIG. 14A) HuHaptoglobin 1-1:Hb complex (50-1.56 nM) (FIG. 14B) HuHaptoglobin2FS(148-406):Hb complex (2000-31.25 nM). (FIG. 14C) HuHemopexin-HuHaptoglobin2FS(148-406):Hb complex (1500-234.4 nM).

FIG. 15 shows vascular function comparing the rescue of NO-dependent vasodilation after the addition of different Hp-variants. The vasodilatory response to NO was measured after the addition of Hb and again after the subsequent addition of a Hb-scavenger. The effect of the Hp-variants (plasma Hp1-1, recHp1-1, recHpCD163low, miniHp, and SuperScavenger) were compared to the benchmark, Hp2-2 (blue; dataset on the left of each window).

FIG. 16 (FIGS. 16A-16B) shows lipid peroxidation comparing the protective effect of different Hp-variants. (FIG. 16A) Generation of MDA in a mixture of Hb with equimolar concentrations of Hp-variants and rLP was measured using fluorescence emission after a 4 hour incubation at 37° C. Hb without any scavenger proteins was used as positive control and rLP alone as negative control. (FIG. 16B) Different Hb scavengers (10 μM) and rLP (2 g/L) were incubated with a range of Hb concentrations (0 to 100 μM) for 4 hours at 37° C. Lipid peroxidation was quantified using a TBARS assay.

FIG. 17 (FIGS. 17A-17B) shows (FIG. 17A) Binding affinity of plasma derived heme:Hx and (FIG. 17B) heme:Hx-Hp complex and uncomplexed (insert) scavenger proteins to biotinylated LRP1 Cluster Ill. The grey lines represent sensorgrams from a range of ligand concentrations in the fluid-phase (all 2000-31.25 nM). The fits are indicated by the red lines.

### DETAILED DESCRIPTION

Unless defined otherwise, all technical and scientific terms used herein have the same meanings as commonly understood by one of ordinary skill in the art to which this invention belongs.

Unless otherwise specified, the indefinite articles “a”, “an” and “the” as used herein, include plural aspects. Thus, for example, reference to “an agent” includes a single agent, as well as two or more agents; reference to “the composition” includes a single composition, as well as two or more compositions; and so forth.

As used herein, the term “about” means±10% of the recited value.

Throughout this specification and the claims that follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The term “consisting of” means “consisting only of”, that is, including and limited to the integer or step or group of integers or steps, and excluding any other integer or step or group of integers or steps.

The term “consisting essentially of means the inclusion of the stated integer or step or group of integers or steps, but other integer or step or group of integers or steps that do not materially alter or contribute to the working of the invention may also be included.

In the absence of any indication to the contrary, reference made to a “%” content throughout this specification is to be taken as meaning % w/w (weight/weight). For example, a solution comprising a haptoglobin content of at least 80% of total protein is taken to mean a composition comprising a haptoglobin content of at least 80% w/w of total protein.

As noted elsewhere herein, the present invention is predicated, at least in part, on the inventors' surprising finding that a functional haptoglobin beta chain, or a haemoglobin-binding fragment thereof, can be produced in a mammalian expression system from an N-terminal truncated pro-haptoglobin (proHp). Advantageously, the N-terminal truncated proHp may be modified to carry a functional moiety, such as Hpx, Fc and albumin, thereby producing a construct with improved therapeutic properties. Moreover, the inventors have unexpectedly found that the expression system described herein advantageously results in stable transfection and expression of a functional haptoglobin β-chain and is therefore distinguished from existing expression systems that achieve, at best, transient transfection and generally fail to express a functional Hp β-chain. The expression system described herein also advantageously allows for the generation of fusion proteins or conjugates, including where a fusion partner could be placed N-terminal to the β-chain fragment and conveniently linked to an inter cysteine residue via a disulphide bond.

Thus, in one aspect disclosed herein, there is provided an expression system for producing a recombinant haptoglobin beta chain, or a haemoglobin-binding fragment thereof, in a mammalian cell, the expression system comprising:
(a) a first nucleic acid sequence encoding an N-terminal truncated pro-haptoglobin (proHp), wherein the N-terminal truncated proHp comprises (i) at least 14 contiguous C-terminal amino acid residues of a haptoglobin alpha chain and (ii) a haptoglobin beta chain, or a haemoglobin-binding fragment thereof, and wherein the N-terminal truncated proHp comprises an internal enzymatic cleavage site between the at least 14 contiguous C-terminal amino acid residues of a haptoglobin alpha chain and the haptoglobin beta chain, or haemoglobin-binding fragment thereof, and
(b) a second nucleic acid sequence encoding an enzyme capable of cleaving the N-terminal truncated proHp at the enzymatic cleavage site;
   wherein, upon introduction of the first nucleic acid sequence and the second nucleic acid sequence into a mammalian cell, and subsequent expression of the N-terminal truncated proHp and the enzyme in the cell, the enzyme is capable of cleaving the N-terminal truncated proHp at the internal enzymatic cleavage site, thereby releasing the haptoglobin beta chain, or haemoglobin-binding fragment thereof, from the N-terminal truncated proHp.

### N-Terminal Truncated Pro-Haptoglobin

Haptoglobin (Hp) is an abundant plasma protein, which is primarily synthesized in the liver. It is a high affinity scavenger for free hemoglobin (Hb) that is occasionally released from erythrocytes during hemolysis. The complex that is formed between the two proteins (Hb:Hp complex) provides a number of protective activities, which attenuate the toxic impact of free Hb in the kidney, the vasculature and in surrounding tissues accessible to free Hb. The protection provided by Hp attenuates two main toxicological consequences of Hb. First, the large molecular size of the Hb:Hp complex prevents extravasation of free Hb. This mechanism protects renal function and preserves vascular nitric oxide (NO) homeostasis by limiting access of free Hb into the vascular wall. Secondly, Hb:Hp complex formation stabilizes the structure of the Hb molecule in a way that limits transfer of heme from its globin chains to proteins and reactive lipids. These mechanisms are largely responsible for Hp's anti-oxidative function during hemolysis. Hp has also been shown to play a role in immune response of T cells, regulation of cell proliferation, angiogenesis, and arterial restructuring.

Hp is synthesized as a single polypeptide precursor, pro-haptogoblin (proHp), which is proteolytically processed by the protease C1rLP (Krzysztof and Fries, PNAS, 2004 101(40):14390-14395). Prohaptoglobin (proHp) is the primary translation product of the Hp mRNA. In the endoplasmic reticulum, proHp dimerizes via disulphide bond formation and is proteolytically cleaved by the protease complement C1r subcomponent-like protein (C1r-LP). As a result, Hp exists in most mammals as a dimeric protein of 150 kDa consisting of two light α-chains and two heavy β-chains that are linked by a single disulphide bond (S—S) between the two α-chains. The Hp protein of most mammals is composed of two (αβ)-monomers linked together via an interface between the two α-chains generating an (αβ)2 structure (termed Hp1-1 in humans). Three Hp phenotypes exist in humans due to the presence of two Hp gene alleles, designated Hp1 and Hp2. The Hp2 allele which arose by an intragenic duplication of the Hp1 allele encodes a slightly larger α-chain but is otherwise identical to the Hp1 allele. Because the cysteine residue connecting the α-chains is duplicated in the encoded Hp2 protein, the Hp2-1 and Hp2-2 phenotypes display a spectrum of various Hp (αβ)-multimers. Haptoglobin-haemoglobin consists of a dimer of haptoglobin chains, each interacting with an αβ dimer of haemoglobin. At each end the β-chain of haptoglobin forms a stable complex with a haemoglobin dimer. Interactions with the clearance receptor CD163 are also mediated by the β-chain.

The major functions of Hp (i.e., binding to Hb and to CD163) are mediated by the β-chain which is encoded by amino acid residues corresponding to amino acid residues 162-406 of the human proHp as shown in SEQ ID NO:1. However, the recombinant expression of a construct encoding these amino acid sequences in mammalian cells does not result in the expression of a protein product. It has now been surprisingly found by the present inventors that, by introducing at least an additional 14 amino acids N-terminal to the proteolytic cleavage site of proHp and co-expressing this construct with a serine protease, robust expression of the Hp β-chain can be achieved that retains Hb and CD163 binding. The inventors have also surprisingly found that the N-terminal truncated proHp can be modified by conjugating or linking the β-chain component of the N-terminal truncated proHp to a functional moiety, such as Fc, albumin or hemopexin (Hpx), and yet still generate the modified construct in relatively high yields, noting also that the functional moieties retain binding affinity to their respective targets, Hb and heme (and to either CD163 or CD91 in the case of Hpx fusion proteins).

The term “N-terminal truncated proHp” is to be understood to mean a fragment of proHp having an amino acid sequence that is shorter than the length of a native (naturally-occurring) proHp molecule by virtue of a truncated N-terminus that would otherwise form part of the complete Hp α-chain. The proHp may be truncated at its N-terminus by any number of amino acid residues, as long as the N-terminal truncated proHp retains at least 14 contiguous C-terminal amino acid residues of the Hp α-chain. In an embodiment, the expressed N-terminal truncated proHp comprises a disulphide bond between the 14 contiguous C-terminal amino acid residues of the Hp α-chain and the Hp β-chain. In an embodiment, the N-terminal truncated proHp comprises a disulphide bond between a cysteine residue within the at least 14 contiguous C-terminal amino acid residues of the haptoglobin alpha chain and at a position corresponding to amino acid position 266 of SEQ ID NO:1. In an embodiment, the N-terminal truncated proHp comprises cysteine residues at positions corresponding to amino acid positions 149 and 266 of the human proHp as shown in SEQ ID NO:1.

It is to be understood that the present disclosure is not limited to N-terminal truncated proHp of a specific amino acid sequence or encoded by a specific nucleic acid sequence, and that any suitable N-terminal truncated proHp can be used in accordance with the present invention, as long as the N-terminal truncated proHp suitably comprises:
(i) at least 14 contiguous C-terminal amino acid residues of a haptoglobin alpha chain;
(ii) a haptoglobin beta chain, or a haemoglobin-binding fragment thereof; and
(iii) an internal enzymatic cleavage site between the at least 14 contiguous C-terminal amino acid residues of a haptoglobin alpha chain and the haptoglobin beta chain, or haemoglobin-binding fragment thereof.

Suitable amino acid sequences of the Hp α-chain will be familiar to persons skilled in the art, illustrative examples of which include amino acid residues 19-160 of SEQ ID NO:1, amino acid residues 19-100 of SEQ ID NO:2 and amino acid residues 19-101 of SEQ ID NO:3. In an embodiment, the at least 14 contiguous C-terminal amino acid residues of a haptoglobin alpha chain comprises, consists or consists essentially of an amino acid sequence having at least 80% (e.g., 80%, 81%, 82%, 83%, 84%, 85%, 86%, 87%, 88%, 89%, 90%, 91%, 92%, 93%, 94%, 95%, 96%, 97%, 98%, 99% or 100%) sequence identity to amino acid residues 148-161 of SEQ ID NO:1 (i.e., VCGKPKNPANPVQR; SEQ ID NO:8).

Suitable amino acid sequences of the Hp β-chain, including of the region of Hp β-chain capable of binding Hb and CD163, will also be familiar to persons skilled in the art, illustrative examples of which include amino acid residues 162-406 of SEQ ID NO:1 (human proHp isoform 1; proHp1), amino acid residues 102-340 of SEQ ID NO:2 (human proHp isoform 2; proHp2) and amino acid residues 103-343 of SEQ ID NO:3 (human proHp isoform 3; proHp3). In an embodiment, the Hp β-chain comprises, consists or consists essentially of an amino acid sequence having at least 80% (e.g., 80%, 81%, 82%, 83%, 84%, 85%, 86%, 87%, 88%, 89%, 90%, 91%, 92%, 93%, 94%, 95%, 96%, 97%, 98%, 99% or 100%) sequence identity to amino acid residues 162-406 of SEQ ID NO:1. In an embodiment, the Hp β-chain comprises, consists or consists essentially of an amino acid sequence having at least 80% (e.g., 80%, 81%, 82%, 83%, 84%, 85%, 86%, 87%, 88%, 89%, 90%, 91%, 92%, 93%, 94%, 95%, 96%, 97%, 98%, 99% or 100%) sequence identity to amino acid residues 102-340 of SEQ ID NO:2. In an embodiment, the Hp β-chain comprises, consists or consists essentially of an amino acid sequence having at least 80% (e.g., 80%, 81%, 82%, 83%, 84%, 85%, 86%, 87%, 88%, 89%, 90%, 91%, 92%, 93%, 94%, 95%, 96%, 97%, 98%, 99% or 100%) sequence identity to amino acid residues 103-343 of SEQ ID NO:3. In an embodiment, the Hp β-chain comprises, consists or consists essentially of an amino acid sequence having at least 85% (e.g., 85%, 86%, 87%, 88%, 89%, 90%, 91%, 92%, 93%, 94%, 95%, 96%, 97%, 98%, 99% or 100%) sequence identity to amino acid residues 162-406 of SEQ ID NO:1. In an embodiment, the Hp β-chain comprises, consists or consists essentially of an amino acid sequence having at least 85% (e.g., 85%, 86%, 87%, 88%, 89%, 90%, 91%, 92%, 93%, 94%, 95%, 96%, 97%, 98%, 99% or 100%) sequence identity to amino acid residues 102-340 of SEQ ID NO:2. In an embodiment, the Hp β-chain comprises, consists or consists essentially of an amino acid sequence having at least 85% (e.g., 85%, 86%, 87%, 88%, 89%, 90%, 91%, 92%, 93%, 94%, 95%, 96%, 97%, 98%, 99% or 100%) sequence identity to amino acid residues 103-343 of SEQ ID NO:3. In an embodiment, the Hp β-chain comprises, consists or consists essentially of an amino acid sequence having at least 90% (e.g., 90%, 91%, 92%, 93%, 94%, 95%, 96%, 97%, 98%, 99% or 100%) sequence identity to amino acid residues 162-406 of SEQ ID NO:1. In an embodiment, the Hp β-chain comprises, consists or consists essentially of an amino acid sequence having at least 90% (e.g., 90%, 91%, 92%, 93%, 94%, 95%, 96%, 97%, 98%, 99% or 100%) sequence identity to amino acid residues 102-340 of SEQ ID NO:2. In an embodiment, the Hp β-chain comprises, consists or consists essentially of an amino acid sequence having at least 90% (e.g., 90%, 91%, 92%, 93%, 94%, 95%, 96%, 97%, 98%, 99% or 100%) sequence identity to amino acid residues 103-343 of SEQ ID NO:3.

In an embodiment, the Hp β-chain comprises, consists or consists essentially of an amino acid sequence having at least 95% (e.g., 95%, 96%, 97%, 98%, 99% or 100%) sequence identity to amino acid residues 162-406 of SEQ ID NO:1. In an embodiment, the Hp β-chain comprises, consists or consists essentially of an amino acid sequence having at least 95% (e.g., 95%, 96%, 97%, 98%, 99% or 100%) sequence identity to amino acid residues 102-340 of SEQ ID NO:2. In an embodiment, the Hp β-chain comprises, consists or consists essentially of an amino acid sequence having at least 95% (e.g., 95%, 96%, 97%, 98%, 99% or 100%) sequence identity to amino acid residues 103-343 of SEQ ID NO:3.

It will also be understood by persons skilled in the art that, in some instances, the sequence of the N-terminal truncated proHp that is selected will likely depend on the intended use, including the intended therapeutic use. By way of example, where the recombinant Hp is to be used for the treatment and/or prevention of a condition in a human subject, the amino acid sequence of the N-terminal truncated proHp will advantageously be derived from a human proHp, including because it minimises the likelihood that the administration of the recombinant Hp to a subject will generate antibodies to the recombinant Hp that would otherwise reduce its efficacy in vivo. Similarly, where the recombinant Hp is to be used for the treatment and/or prevention of a condition in a non-human subject, such as for veterinary applications, the amino acid sequence of the N-terminal truncated proHp will advantageously be derived from a proHp isoform that is native to a non-human subject. Suitable non-human isoforms of proHp will be familiar to persons skilled in the art, illustrative examples of which include canine, feline, equine, bovine, ovine and primate proHp. Illustrative examples of primate proHp are described in GenBank Accession Nos. AFH32200 and JAB04820. In an embodiment, the N-terminal truncated proHp has an amino acid sequence derived from a human N-terminal truncated proHp. Thus, in an embodiment, the haptoglobin is a human haptoglobin. Suitable human proHp amino acid sequences will be familiar to persons skilled in the art, illustrative examples of which include those described in GenBank Accession Nos. NP_005134 (human Hp isoform 1 precursor proHp; SEQ ID NO:1; also referred to as isoform Hp1 or Hp1F), NP_001119574 (human Hp isoform 2 precursor proHp; SEQ ID NO:2; also referred to as isoform Hp2 or Hp2SS) and NP_001305067 (human Hp isoform 3 precursor proHp; SEQ ID NO:3; also referred to as isoform Hp3). Subtypes of human Hp isoforms will also be known to persons skilled in the art, illustrative examples of which include (i) Hp1F (SEQ ID NO:1), comprising residues Asp and Lys at amino acid positions 70 and 71, respectively, as shown in SEQ ID NO:1; (ii) Hp1S comprising residues Asn and Glu at positions corresponding to amino acid positions 70 and 71, respectively, of SEQ ID NO:1; (iii) Hp2SS (SEQ ID NO:2) comprising residues Asn and Glu at amino acid positions at 70 and 71, respectively, and residues Asn and Glu at amino acid positions 129 and 130, respectively, as shown in SEQ ID NO:2; and (iv) Hp2FS comprising residues Asp and Lys at positions corresponding to amino acid positions 70 and 71, respectively, and residues Asn and Glu at positions corresponding to amino acid positions 129 and 130, respectively, of SEQ ID NO:2.

In an embodiment, the haptoglobin is a human haptoglobin isoform Hp1F, as described herein. In another embodiment, the haptoglobin is a human haptoglobin isoform Hp1S, as described herein. In another embodiment, the haptoglobin is a human haptoglobin isoform Hp2FS, as described herein. In another embodiment, the haptoglobin is a human haptoglobin isoform Hp2SS, as described herein.

In an embodiment, the proHp comprises, consists or consists essentially of an amino acid sequence having at least 80% (e.g., 80%, 81%, 82%, 83%, 84%, 85%, 86%, 87%, 88%, 89%, 90%, 91%, 92%, 93%, 94%, 95%, 96%, 97%, 98%, 99% or 100%) sequence identity to SEQ ID NO:1. In an embodiment, the proHp comprises, consists or consists essentially of an amino acid sequence having at least 85% (e.g., 85%, 86%, 87%, 88%, 89%, 90%, 91%, 92%, 93%, 94%, 95%, 96%, 97%, 98%, 99% or 100%) sequence identity to SEQ ID NO:1. In an embodiment, the proHp comprises, consists or consists essentially of an amino acid sequence having at least 90% (e.g., 90%, 91%, 92%, 93%, 94%, 95%, 96%, 97%, 98%, 99% or 100%) sequence identity to SEQ ID NO:1. In an embodiment, the proHp comprises, consists or consists essentially of an amino acid sequence having at least 95% (e.g., 95%, 96%, 97%, 98%, 99% or 100%) sequence identity to SEQ ID NO:1. In an embodiment, the proHp comprises, consists or consists essentially of an amino acid sequence having at least 80% (e.g., 80%, 81%, 82%, 83%, 84%, 85%, 86%, 87%, 88%, 89%, 90%, 91%, 92%, 93%, 94%, 95%, 96%, 97%, 98%, 99% or 100%) sequence identity to SEQ ID NO:2. In an embodiment, the proHp comprises, consists or consists essentially of an amino acid sequence having at least 85% (e.g., 85%, 86%, 87%, 88%, 89%, 90%, 91%, 92%, 93%, 94%, 95%, 96%, 97%, 98%, 99% or 100%) sequence identity to SEQ ID NO:2. In an embodiment, the proHp comprises, consists or consists essentially of an amino acid sequence having at least 90% (e.g., 90%, 91%, 92%, 93%, 94%, 95%, 96%, 97%, 98%, 99% or 100%) sequence identity to SEQ ID NO:2. In an embodiment, the proHp comprises, consists or consists essentially of an amino acid sequence having at least 95% (e.g., 95%, 96%, 97%, 98%, 99% or 100%) sequence identity to SEQ ID NO:2. In an embodiment, the proHp comprises, consists or consists essentially of an amino acid sequence having at least 80% (e.g., 80%, 81%, 82%, 83%, 84%, 85%, 86%, 87%, 88%, 89%, 90%, 91%, 92%, 93%, 94%, 95%, 96%, 97%, 98%, 99% or 100%) sequence identity to SEQ ID NO:3. In an embodiment, the proHp comprises, consists or consists essentially of an amino acid sequence having at least 85% (e.g., 85%, 86%, 87%, 88%, 89%, 90%, 91%, 92%, 93%, 94%, 95%, 96%, 97%, 98%, 99% or 100%) sequence identity to SEQ ID NO:3. In an embodiment, the proHp comprises, consists or consists essentially of an amino acid sequence having at least 90% (e.g., 90%, 91%, 92%, 93%, 94%, 95%, 96%, 97%, 98%, 99% or 100%) sequence identity to SEQ ID NO:3. In an embodiment, the proHp comprises, consists or consists essentially of an amino acid sequence having at least 95% (e.g., 95%, 96%, 97%, 98%, 99% or 100%) sequence identity to SEQ ID NO:3.

In an embodiment, the N-terminal truncated proHp comprises, consists or consists essentially of an amino acid sequence having at least 80% (e.g., 80%, 81%, 82%, 83%, 84%, 85%, 86%, 87%, 88%, 89%, 90%, 91%, 92%, 93%, 94%, 95%, 96%, 97%, 98%, 99% or 100%) sequence identity to amino acid residues 148 to 406 of SEQ ID NO:1. In an embodiment, the N-terminal truncated proHp comprises, consists or consists essentially of an amino acid sequence having at least 85% (e.g., 85%, 86%, 87%, 88%, 89%, 90%, 91%, 92%, 93%, 94%, 95%, 96%, 97%, 98%, 99% or 100%) sequence identity to amino acid residues 148 to 406 of SEQ ID NO:1. In an embodiment, the N-terminal truncated proHp comprises, consists or consists essentially of an amino acid sequence having at least 90% (e.g., 90%, 91%, 92%, 93%, 94%, 95%, 96%, 97%, 98%, 99% or 100%) sequence identity to amino acid residues 148 to 406 of SEQ ID NO:1. In an embodiment, the N-terminal truncated proHp comprises, consists or consists essentially of an amino acid sequence having at least 95% (e.g., 95%, 96%, 97%, 98%, 99% or 100%) sequence identity to amino acid residues 148 to 406 of SEQ ID NO:1.

In an embodiment, the N-terminal truncated proHp comprises, consists or consists essentially of an amino acid sequence having at least 80% (e.g., 80%, 81%, 82%, 83%, 84%, 85%, 86%, 87%, 88%, 89%, 90%, 91%, 92%, 93%, 94%, 95%, 96%, 97%, 98%, 99% or 100%) sequence identity to amino acid residues 89 to 347 of SEQ ID NO:2. In an embodiment, the N-terminal truncated proHp comprises, consists or consists essentially of an amino acid sequence having at least 85% (e.g., 85%, 86%, 87%, 88%, 89%, 90%, 91%, 92%, 93%, 94%, 95%, 96%, 97%, 98%, 99% or 100%) sequence identity to amino acid residues 89 to 347 of SEQ ID NO:2. In an embodiment, the N-terminal truncated proHp comprises, consists or consists essentially of an amino acid sequence having at least 90% (e.g., 90%, 91%, 92%, 93%, 94%, 95%, 96%, 97%, 98%, 99% or 100%) sequence identity to amino acid residues 89 to 347 of SEQ ID NO:2. In an embodiment, the N-terminal truncated proHp comprises, consists or consists essentially of an amino acid sequence having at least 95% (e.g., 95%, 96%, 97%, 98%, 99% or 100%) sequence identity to amino acid residues 89 to 347 of SEQ ID NO:2.

In an embodiment, the N-terminal truncated proHp comprises, consists or consists essentially of an amino acid sequence having at least 80% (e.g., 80%, 81%, 82%, 83%, 84%, 85%, 86%, 87%, 88%, 89%, 90%, 91%, 92%, 93%, 94%, 95%, 96%, 97%, 98%, 99% or 100%) sequence identity to amino acid residues 89 to 347 of SEQ ID NO:3. In an embodiment, the N-terminal truncated proHp comprises, consists or consists essentially of an amino acid sequence having at least 85% (e.g., 85%, 86%, 87%, 88%, 89%, 90%, 91%, 92%, 93%, 94%, 95%, 96%, 97%, 98%, 99% or 100%) sequence identity to amino acid residues 89 to 347 of SEQ ID NO:3. In an embodiment, the N-terminal truncated proHp comprises, consists or consists essentially of an amino acid sequence having at least 90% (e.g., 90%, 91%, 92%, 93%, 94%, 95%, 96%, 97%, 98%, 99% or 100%) sequence identity to amino acid residues 89 to 347 of SEQ ID NO:3. In an embodiment, the N-terminal truncated proHp comprises, consists or consists essentially of an amino acid sequence having at least 95% (e.g., 95%, 96%, 97%, 98%, 99% or 100%) sequence identity to amino acid residues 89 to 347 of SEQ ID NO:3.

Reference to “at least 80%” includes 80, 81, 82, 83, 84, 85, 86, 87, 88, 89, 90, 91, 92, 93, 94, 95, 96, 97, 98, 99 and 100% sequence identity, for example, after optimal alignment or best fit analysis. Optimal alignment of sequences for aligning a comparison window may be conducted by computerized implementations of algorithms (GAP, BESTFIT, FASTA, and TFASTA in the Wisconsin Genetics Software Package Release 7.0, Genetics Computer Group, 575 Science Drive Madison, WI, USA) or by inspection and the best alignment (i.e., resulting in the highest percentage homology over the comparison window) generated by any of the various methods selected. Reference also may be made to the BLAST family of programs as for example disclosed by Altschul et al. (1997) Nucl. Acids. Res. 25:3389. A detailed discussion of sequence analysis can be found in Unit 19.3 of Ausubel et al. (1994-1998) In: Current Protocols in Molecular Biology, John Wiley & Sons Inc.

The term “sequence identity” as used herein refers to the extent that sequences are identical or structurally similar on a nucleotide-by-nucleotide basis or an amino acid-by-amino acid basis over a window of comparison. Thus, a “percentage of sequence identity”, for example, is calculated by comparing two optimally aligned sequences over the window of comparison, determining the number of positions at which the identical nucleic acid base (e.g., A, T, C, G, I) or the identical amino acid residue (e.g., Ala, Pro, Ser, Thr, Gly, Val, Leu, lie, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity. For example, “sequence identity” is the “match percentage” calculated by the DNASIS computer program (Version 2.5 for windows; available from Hitachi Software engineering Co., Ltd., South San Francisco, California, USA) using standard defaults as used in the reference manual accompanying the software.

The term “sequence identity”, as used herein, includes exact identity between compared sequences at the nucleotide or amino acid level. This term is also used herein to include non-exact identity (i.e., similarity) at the nucleotide or amino acid level where any difference(s) between sequences are in relation to amino acids (or in the context of nucleotides, amino acids encoded by said nucleotides) that are nevertheless related to each other at the structural, functional, biochemical and/or conformational levels. For example, where there is non-identity (similarity) at the amino acid level, “similarity” includes amino acids that are nevertheless related to each other at the structural, functional, biochemical and/or conformational levels. In an embodiment, nucleotide and sequence comparisons are made at the level of identity rather than similarity. For example, leucine may be substituted for an isoleucine or valine residue. This may be referred to as a conservative substitution. In an embodiment, the amino acid sequences may be modified by way of conservative substitution of any of the amino acid residues contained therein, such that the modification has no or negligible effect on the binding specificity or functional activity of the modified polypeptide when compared to the unmodified polypeptide.

Sequence identity, as herein described, typically relates to the percentage of amino acid residues in the candidate sequence that are identical with the residues of the corresponding peptide sequence after aligning the sequences and introducing gaps, if necessary, to achieve the maximum percentage homology, and not considering any conservative substitutions as part of the sequence identity. Neither N- or C- terminal extensions, nor insertions shall be construed as reducing sequence identity or homology.

Functional variants of N-terminal truncated proHp are also contemplated herein. As used herein, the term “functional variant” refers to a peptide that shares at least some amino acid sequence identity with a native (naturally-occurring) isoform of proHp (human or non-human), but still retains the ability to bind to Hb. In this context, the terms “functional variant” and “Hb-binding function variant” are used interchangeably herein. Functional variants extend to a proHp with a truncated C-terminus (i.e., a C-terminal truncated Hp β-chain), although it is to be understood that C-terminal truncated proHp would suitably retain at least part of the Hb-binding region of the Hp β-chain, which would be familiar to persons skilled in the art. Moreover, suitable methods of screening for functional variants comprising C-terminal truncated Hp β-chain that retain Hb binding activity will be familiar to persons skilled in the art, illustrative examples of which are described elsewhere herein, such as surface plasmon resonance (SPR) and size exclusion chromatography (e.g., HPLC). These methods are also described in Schaer et al. (2018; BMC Biotechnol. 18:15), the contents of which are incorporated herein by reference.

The present disclosure also extends to functional variants that differ from the native sequence by one or more amino acid substitutions, including conservative amino acid substitutions, deletions or insertions. In an embodiment, the functional variant comprises an amino acid sequence that differs from a native sequence by way of conservative substitution of any of the amino acid residues contained therein, such that the modification has no or negligible effect on the Hb binding specificity or functional activity of the functional variant when compared to the unmodified (e.g., native) molecule. Suitable methods of screening for functional variants comprising one or more amino acid substitutions, deletions or insertions that retain Hb binding activity will also be familiar to persons skilled in the art, illustrative examples of which are described elsewhere herein.

In some embodiments, the functional variant comprises, consists or consists essentially of an amino acid sequence having at least 60%, preferably at least 65%, preferably at least 70%, preferably at least 75%, preferably at least 80%, preferably at least 85%, preferably at least 90%, preferably at least 93%, preferably at least 95%, preferably at least 96%, preferably at least 97%, preferably at least 98% or preferably at least 99% sequence identity to amino acid residues 148 to 406 of SEQ ID NO:1, amino acid residues 89 to 347 of SEQ ID NO:2 or amino acid residues 89 to 347 of SEQ ID NO:3.

In an embodiment, the N-terminal truncated proHp comprises, consists or consists essentially of an amino acid sequence having at least 80% sequence identity to amino acid residues 148 to 406 of SEQ ID NO:1. In an embodiment, the N-terminal truncated proHp comprises, consists or consists essentially of an amino acid sequence having at least 90% sequence identity to amino acid residues 148 to 406 of SEQ ID NO:1. In an embodiment, the N-terminal truncated proHp comprises, consists or consists essentially of an amino acid sequence having at least 95% sequence identity to amino acid residues 148 to 406 of SEQ ID NO:1. In an embodiment, the N-terminal truncated proHp comprises, consists or consists essentially of amino acid residues 148 to 406 of SEQ ID NO:1.

In an embodiment, the N-terminal truncated proHp comprises, consists or consists essentially of an amino acid sequence having at least 80% sequence identity to amino acid residues 89-347 of SEQ ID NO:2. In an embodiment, the N-terminal truncated proHp comprises, consists or consists essentially of an amino acid sequence having at least 90% sequence identity to amino acid residues 89-347 of SEQ ID NO:2. In an embodiment, the N-terminal truncated proHp comprises, consists or consists essentially of an amino acid sequence having at least 95% sequence identity to amino acid residues 89-347 of SEQ ID NO:2. In an embodiment, the N-terminal truncated proHp comprises, consists or consists essentially of amino acid residues 89-347 of SEQ ID NO:2.

In an embodiment, the N-terminal truncated proHp comprises, consists or consists essentially of an amino acid sequence having at least 80% sequence identity to amino acid residues 89-347 of SEQ ID NO:3. In an embodiment, the N-terminal truncated proHp comprises, consists or consists essentially of an amino acid sequence having at least 90% sequence identity to amino acid residues 89-347 of SEQ ID NO:3. In an embodiment, the N-terminal truncated proHp comprises, consists or consists essentially of an amino acid sequence having at least 95% sequence identity to amino acid residues 89-347 of SEQ ID NO:3. In an embodiment, the N-terminal truncated proHp comprises, consists or consists essentially of amino acid residues 89-347 of SEQ ID NO:2.

In a preferred embodiment, the N-terminal truncated proHp comprises a native internal enzymatic cleavage site between the at least 14 contiguous C-terminal amino acid residues of a haptoglobin alpha chain and the haptoglobin beta chain, or haemoglobin-binding fragment thereof; that is, the internal enzymatic cleavage site is native to the proHp from which the amino acid sequence of the N-terminal truncated proHp derives. In the human proHp isoform 1 (SEQ ID NO:1), the internal enzymatic cleavage site is located at amino acid positions 161 and 162 of SEQ ID NO:1, such that enzymatic cleavage at this site liberates the Hp alpha chain (amino acid residues 19-161 of SEQ ID NO:1) from the Hp beta chain (amino acid residues 162-406 of SEQ ID NO:1). In the human proHp isoform 2 (SEQ ID NO:2), the internal enzymatic cleavage site is located at amino acid positions 162 and 163 of SEQ ID NO:1, such that enzymatic cleavage at this site liberates the Hp alpha chain (amino acid residues 19-162 of SEQ ID NO:2) from the Hp beta chain (corresponding to amino acid residues 163-407 of SEQ ID NO:2). In the human proHp isoform 3 (SEQ ID NO:3), the internal enzymatic cleavage site is located at amino acid positions 162 and 163 of SEQ ID NO:3, such that enzymatic cleavage at this site liberates the Hp alpha chain (amino acid residues 19-162 of SEQ ID NO:3) from the Hp beta chain (corresponding to amino acid residues 163-407 of SEQ ID NO:3).

In other embodiments, the first nucleic acid sequence encoding an N-terminal truncated proHp may comprise an internal enzymatic cleavage site between the at least 14 contiguous C-terminal amino acid residues of a haptoglobin alpha chain and the haptoglobin beta chain, or haemoglobin-binding fragment thereof; that is, the internal enzymatic cleavage site is non-native to the proHp from which the amino acid sequence of the N-terminal truncated proHp derives. In this context, the internal enzymatic cleavage site will be selected such that it is compatible with the enzyme encoded by the second nucleic acid sequence of the expression system, such that the enzyme encoded by the second nucleic acid sequence is capable of cleaving the N-terminal truncated proHp at the non-native enzymatic cleavage site. Suitable non-native internal enzymatic cleavage sites will be familiar to persons skilled in the art, as would their corresponding enzymes. Illustrative examples of suitable non-native internal enzymatic cleavage sites include a furin cleavage site, a non-native serine protease cleavage site, a cysteine protease cleavage site, an aspartic protease cleavage site, a metalloprotease cleavage site, and a threonine protease cleavage site. Thus, in an embodiment disclosed herein, the internal enzymatic cleavage site is selected from the group consisting of a furin cleavage site, a non-native serine protease cleavage site, a cysteine protease cleavage site, an aspartic protease cleavage site, a metalloprotease cleavage site, and a threonine protease cleavage site.

In an embodiment, the internal enzymatic cleavage site is a non-serine protease cleavage site. In a preferred embodiment, the serine protease cleavage site is a C1r-like protein (C1rLP) cleavage site, or a functional variant thereof, as described elsewhere herein.

### Additional Functional Moieties

In the expression system disclosed herein, the N-terminal truncated proHp encoded by the first nucleic acid sequence may further comprise one or more additional functional moieties. In an embodiment, the functional moiety is linked, fused, conjugated, coupled, tethered or otherwise attached to one or more of the at least 14 contiguous C-terminal amino acid residues of the haptoglobin alpha chain. In an embodiment, the additional functional moiety is linked, fused, conjugated, coupled, tethered or otherwise attached to one or more of the amino acid residues of the haptoglobin beta chain. In an embodiment, the additional functional moiety is linked, fused, conjugated, coupled, tethered or otherwise attached to the N-terminal truncated proHp by a disulphide bond at a cysteine residue within the at least 14 contiguous C-terminal amino acid residues. In an embodiment, the additional functional moiety is linked, fused, conjugated, coupled, tethered or otherwise attached to the N-terminal truncated proHp by a disulphide bond at a cysteine residue corresponding to amino acid position corresponding 149 of SEQ ID NO:1.

In some embodiments, the functional moiety may be covalently bound to the N-terminal truncated proHp. In other embodiments, the N-terminal truncated proHp may be fused, coupled or otherwise attached to one or more heterologous moieties as part of a fusion protein. The one or more additional functional moieties will suitably improve, enhance or otherwise extend the activity and/or stability of the haptoglobin beta chain, or the haemoglobin-binding fragment thereof, as described herein.

To facilitate isolation of the recombinant protein, as herein described, a fusion polypeptide may be made where the N-terminal truncated proHp, or functional variant thereof, is translationally fused (covalently linked) to a heterologous polypeptide which enables isolation, such as by affinity chromatography. Suitable heterologous polypeptides would be known to the skilled person, illustrative examples of which include His-Tag (e.g. 8 histidine residues (SEQ ID NO: 18)), GST-Tag (Glutathione-S-transferase), V5 tag, HA-tag, CBP (Chitin Binding Protein)-tag, MBP (Maltose Binding Protein) tag, Streptavidin-tag, SBP (Streptavidin binding protein) Myc-tag and Biotin-tag.

In some embodiments, the N-terminal truncated proHp described herein is suitably attached to a functional moiety for extending the half-life of the recombinant haptoglobin beta chain, or a haemoglobin-binding fragment thereof in vivo. Suitable half-life extending functional moieties will be familiar to persons skilled in the art, illustrative examples of which include polyethylene glycol (PEGylation), glycosylated PEG, hydroxyl ethyl starch (HESylation), polysialic acids, elastin-like polypeptides, heparosan polymers and hyaluronic acid. In an embodiment disclosed herein, functional moiety is selected from the group consisting of polyethylene glycol (PEGylation), glycosylated PEG, hydroxyl ethyl starch (HESylation), polysialic acids, elastin-like polypeptides, heparosan polymers and hyaluronic acid. The half-life extending functional moiety can be linked (e.g., fused, conjugated, tethered or otherwise attached) to the N-terminal truncated proHp, or functional variant thereof, by any suitable means known to persons skilled in the art, an illustrative example of which is via a chemical linker, as described, for example, in U.S. Pat. No. 7,256,253), the entire contents of which are incorporated herein by reference.

In other embodiments, the functional moiety is a half-life enhancing protein (HLEP). Suitable half-life enhancing proteins will be familiar to persons skilled in the art, illustrative examples of which includes albumin and fragments thereof. Thus, in an embodiment, the HLEP is an albumin or a fragment thereof. The N-terminus of the albumin or fragment thereof may be linked, fused, conjugated, coupled, tethered or otherwise attached to the C-terminus of the alpha and/or beta chains of the N-terminal truncated proHp. Alternatively, or in addition, the C-terminus of the albumin or fragment thereof may linked, fused, conjugated, coupled, tethered or otherwise attached to the N-terminus of the alpha and/or beta chains of the N-terminal truncated proHp. One or more HLEPs may be fused to the N- or C-terminal part(s) of the alpha and/or beta chains of the N-terminal truncated proHp provided that they do not abolish the ability of the recombinant haptoglobin beta chain, or a haemoglobin-binding fragment thereof, to bind to cell-free Hb. It is to be understood, however, that some reduction in the binding of the recombinant haptoglobin beta chain, or a haemoglobin-binding fragment thereof, to cell-free Hb may be acceptable, as long as it is still capable of forming a complex with, and thereby neutralise, cell-free Hb.

The terms, “human serum albumin” (HSA) and “human albumin” (HA) and “albumin” (ALB) are used interchangeably herein. The terms “albumin” and “serum albumin” are broader and encompass human serum albumin (and fragments and variants thereof), as well as albumin from other species (and fragments and variants thereof).

As used herein, “albumin” refers collectively to albumin polypeptide or amino acid sequence, or an albumin fragment or variant, having one or more functional activities (e.g., biological activities) of albumin. In particular, “albumin” refers to human albumin or fragments thereof, including the mature form of human albumin or albumin from other vertebrates or fragments thereof, or analogs or variants of these molecules or fragments thereof.

The fusion proteins described herein may suitably comprise naturally-occurring polymorphic variants of human albumin and/or fragments of human albumin. Generally speaking, an albumin fragment or variant will be at least 10, preferably at least 40, or most preferably more than 70 amino acids in length.

In an embodiment, the HLEP is an albumin variant with enhanced binding to the FcRn receptor. Such albumin variants may lead to a longer plasma half-life of the Hp or functional analogue thereof compared to the Hp or functional fragment thereof that is fused to a wild-type albumin. The albumin portion of the fusion proteins described herein may suitably comprise at least one subdomain or domain of human albumin or conservative modifications thereof.

In some embodiments, a linker sequence may be positioned between the N-terminal truncated proHp and the functional moiety. The linker sequence may be a peptidic linker consisting of one or more amino acids, in particular of 1 to 50, preferably 1 to 30, preferably 1 to 20, preferably 1 to 15, preferably 1 to 10, preferably 1 to 5 or more preferably 1 to 3 (e.g. 1, 2 or 3) amino acids and which may be equal or different from each other. Preferred amino acids present in said linker sequence include Gly and Ser. In a preferred embodiment, the linker sequence is substantially non-immunogenic to the subject to be treated in accordance with the methods disclosed herein. By substantially non-immunogenic is meant that the linker sequence will not raise a detectable antibody response to the linker sequence or to the recombinant haptoglobin beta chain, or haemoglobin-binding fragment thereof, in the subject to which it is administered. Preferred linkers may be comprised of alternating glycine and serine residues. Suitable linkers will be familiar to persons skilled in the art, illustrative examples of which are described in WO2007/090584. In an embodiment, the peptidic linker between the N-terminal truncated proHp and the functional moiety comprises, consists or consists essentially of peptide sequences, which serve as natural interdomain linkers in human proteins. Such peptide sequences in their natural environment may be located close to the protein surface and are accessible to the immune system so that one can assume a natural tolerance against this sequence. Illustrative examples are given in WO 2007/090584. Suitable cleavable linker sequences are described, e.g., in WO 2013/120939.

Illustrative examples of suitable HLEP sequences are described infra. Likewise disclosed herein are fusions to the exact “N-terminal amino acid” or to the exact “C-terminal amino acid” of the respective HLEP, or fusions to the “N-terminal part” or “C-terminal part” of the respective HLEP, which includes N-terminal deletions of one or more amino acids of the HLEP. The fusion protein may comprise more than one HLEP sequence, e.g. two or three HLEP sequences. These multiple HLEP sequences may be fused to the C-terminal part of the alpha and/or beta chains of the Hp in tandem, e.g. as successive repeats.

The HLEP portion of the fusion protein, as descried herein, may be a variant of a wild type HELP. The term “variant” when used in relation to the HELP portion of the fusion protein is to be understood to include insertions, deletions and/or substitutions, either conservative or non-conservative, where such changes do not substantially alter the ability of the recombinant haptoglobin beta chain, or a haemoglobin-binding fragment thereof, to form a complex with, and thereby neutralise, cell-free Hb. The HLEP may suitably be derived from any vertebrate, especially any mammal, for example human, monkey, cow, sheep, or pig. Non-mammalian HLEPs include, but are not limited to, hen and salmon.

In an embodiment, the functional moiety is a half-life extending polypeptide. In an embodiment, the half-life extending polypeptide is selected from the group consisting of albumin, a member of the albumin-family or fragments thereof, hemopexin, solvated random chains with large hydrodynamic volume (e.g. XTEN (see Schellenberger et al. 2009; Nature Biotechnol. 27:1186-1190), homo-amino acid repeats (HAP) or proline-alanine-serine repeats (PAS), afamin, alpha-fetoprotein, Vitamin D binding protein, transferrin or variants or fragments thereof, carboxyl-terminal peptide (CTP) of human chorionic gonadotropin-β subunit, a polypeptide capable of binding to the neonatal Fc receptor (FcRn), in particular an immunoglobulin constant region and portions thereof, e.g. the Fc fragment, polypeptides or lipids capable of binding under physiological conditions to albumin, to a member of the albumin-family or to fragments thereof or to an immunoglobulin constant region or portions thereof. In an embodiment, the immunoglobulin constant region or portion thereof is an Fc fragment of immunoglobulin G1 (IgG1), an Fc fragment of immunoglobulin G2 (IgG2), an Fc fragment of immunoglobulin A (IgA), or Fc receptor binding fragments thereof. A half-life enhancing polypeptide, as used herein, may be a full-length half-life-enhancing protein or one or more fragments thereof that are capable of stabilizing or prolonging the therapeutic activity or the biological activity of the recombinant haptoglobin beta chain, or a haemoglobin-binding fragment thereof, in particular of increasing the in vivo half-life of the recombinant haptoglobin beta chain, or a haemoglobin-binding fragment thereof. Such fragments may be of 10 or more amino acids in length or may include at least about 15, preferably at least about 20, preferably at least about 25, preferably at least about 30, preferably at least about 50, or more preferably at least about 100, or more contiguous amino acids from the HLEP sequence, or may include part or all of specific domains of the respective HLEP, as long as the HLEP fragment provides a functional half-life extension of at least 10%, preferably of at least 20%, or more preferably of at least 25%, compared to the respective Hp in the absence of the HLEP. Methods of determining whether a functional moiety provides a functional half-life extension to the recombinant haptoglobin beta chain, or a haemoglobin-binding fragment thereof (in vivo or in vitro) will be familiar to persons skilled in the art, illustrative examples of which are described elsewhere herein.

Conjugates and fusion proteins, as described herein, can be created by in-frame joining of at least two DNA sequences encoding the N-terminal truncated proHp and the one or more functional moieties, such as a HLEP. Persons skilled in the art will understand that translation of the DNA sequence encoding the conjugate or fusion protein will result in a single peptide sequence. As a result of an in-frame insertion of a DNA sequence encoding a peptidic linker according to embodiments disclosed herein, a conjugate or fusion protein comprising the recombinant haptoglobin beta chain, or a haemoglobin-binding fragment thereof, a suitable linker and the functional moiety can be obtained.

In an embodiment disclosed herein, the functional moiety comprises, consists or consists essentially of a polypeptide selected from the group consisting of albumin or fragments thereof, hemopexin, transferrin or fragments thereof, the C-terminal peptide of human chorionic gonadotropin, an XTEN sequence, homo-amino acid repeats (HAP), proline-alanine-serine repeats (PAS), afamin, alpha-fetoprotein, Vitamin D binding protein, polypeptides capable of binding under physiological conditions to albumin or to immunoglobulin constant regions, polypeptides capable of binding to the neonatal Fc receptor (FcRn), particularly immunoglobulin constant regions and portions thereof, preferably the Fc portion of immunoglobulin, and combinations of any of the foregoing. In another embodiment, the functional moiety is selected from the group consisting of hydroxyethyl starch (HES), polyethylene glycol (PEG), polysialic acids (PSAs), elastin-like polypeptides, heparosan polymers, hyaluronic acid and albumin binding ligands, e.g. fatty acid chains, and combinations of any of the foregoing.

In an embodiment, the functional moiety is hemopexin or a heme-binding fragment thereof. Hemopexin is a 61-kDa plasma β-1B-glycoprotein composed of a single 439 amino acids long peptide chain, which is formed by two four-bladed p-propeller domains, resembling two thick disks that lock together at a 90° angle and are joined by an interdomain linker peptide. The heme, which is released into the blood as the result of intra- and extra-vascular haemolysis, is bound between the two four-bladed β-propeller domains in a pocket formed by the interdomain linker peptide. Residues His213 and His266 coordinate the heme iron atom giving a stable bis-histidyl complex, similar to haemoglobin. The term “heme-binding fragment” is to be understood as meaning a fragment of a native hemopexin molecule comprising a sufficient number of contiguous or non-contiguous amino acid residues of a native hemopexin molecule such that it retains at least some of the binding affinity to cell-free heme as the native molecule. Suitable methods for determining whether a fragment of hemopexin retains heme-binding activity will be familiar to persons skilled in the art, illustrative examples are described elsewhere herein. In an embodiment, the heme-binding fragment of hemopexin comprises, consists or consists essentially of an amino acid sequence having at least 50%, preferably at least 55%, preferably at least 60%, preferably at least 65%, preferably at least 70%, preferably at least 75%, preferably at least 80%, preferably at least 85%, preferably at least 90%, or more preferably at least 95% sequence identity to a native hemopexin protein. In an embodiment, the hemopexin is a human hemopexin. In an embodiment, the human hemopexin comprises, consists or consists essentially of an amino acid sequence as shown in NP_000604 (SEQ ID NO:12).

Hemopexin contains about 20% carbohydrates, including sialic acid, mannose, galactose, and glucosamine. Twelve cysteine residues were found in the protein sequence, probably accounting for six disulphide bridges. Hemopexin represents the primary line of defense against heme toxicity thanks to its ability to bind heme with high affinity (K_{d}<1 pM) and to function as a heme specific carrier from the bloodstream to the liver. It binds heme in an equimolar ratio, but there is no evidence that heme is covalently bound to the protein. In addition to heme binding, hemopexin preparations have also been reported to possess serine protease activity (Lin et. al., 2016; Molecular Medicine 22:22-31) and several other functions, such as exhibition of anti- and pro-inflammatory activities, inhibition of cellular adhesion and binding of certain divalent metal ions. Whilst endogenous hemopexin can control the adverse effects of free heme under physiological steady-state conditions, it has little effect in maintaining steady-state heme levels under pathophysiogical conditions, such as those associated with haemolysis, where a high level of heme leads to the depletion of endogenous hemopexin, causing heme-mediated oxidative tissue damage. Studies have shown that hemopexin infusion alleviates heme-induced endothelial activation, inflammation, and oxidative injury in experimental mouse models of haemolytic disorders, such as sickle-cell disease (SCD) and β-thalassemia. Hemopexin administration has also been shown to significantly reduce the level of proinflammatory cytokines and counteract heme-induced vasoconstriction in haemolytic animals.

In an embodiment, the functional moiety is an immunoglobulin molecule comprising an Fc region, or an FcRn-binding fragment thereof. Immunoglobulin Fc regions (Fc) are known in the art to increase the half-life of therapeutic proteins (see, e.g., Dumont J A et al. 2006. BioDrugs 20:151-160). The IgG constant region of the heavy chain consists of 3 domains (CH1-CH3) and a hinge region. The immunoglobulin sequence may be derived from any mammal, or from subclasses IgG1, IgG2, IgG3 or IgG4, respectively. IgG and IgG fragments without an antigen-binding domain may also be used as a functional moiety, including as a HLEP. The Hp or functional analogue thereof may suitably be connected to the IgG or the IgG fragments via the hinge region of the antibody or a peptidic linker, which may even be cleavable. Several patents and patent applications describe the fusion of therapeutic proteins to immunoglobulin constant regions to enhance the therapeutic proteins' in vivo half-lives. For example, US 2004/0087778 and WO 2005/001025 describe fusion proteins of Fc domains or at least portions of immunoglobulin constant regions with biologically active peptides that increase the half-life of the peptide, which otherwise would be quickly eliminated in vivo. Fc-IFN-p fusion proteins were described that achieved enhanced biological activity, prolonged circulating half-life and greater solubility (WO 2006/000448 A2). Fc-EPO proteins with a prolonged serum half-life and increased in vivo potency were disclosed (WO 2005/063808 A1) as well as Fc fusions with G-CSF (WO 2003/076567 A2), glucagon-like peptide-1 (WO 2005/000892 A2), clotting factors (WO 2004/101740 A2) and interleukin-10 (U.S. Pat. No. 6,403,077), all with half-life enhancing properties.

Illustrative examples of suitable HLEP which can be used in accordance with the present invention are also described in WO 2013/120939 A1, the contents of which are incorporated herein by reference in their entirety.

### Expression System

As noted elsewhere, the present disclosure provides a mammalian expression system comprising:
(a) a first nucleic acid sequence encoding an N-terminal truncated pro-haptoglobin (proHp), wherein the N-terminal truncated proHp comprises (i) at least 14 contiguous C-terminal amino acid residues of a haptoglobin alpha chain and (ii) a haptoglobin beta chain, or a haemoglobin-binding fragment thereof, and wherein the N-terminal truncated proHp comprises an internal enzymatic cleavage site between the at least 14 contiguous C-terminal amino acid residues of a haptoglobin alpha chain and the haptoglobin beta chain, or haemoglobin-binding fragment thereof, and
(b) a second nucleic acid sequence encoding an enzyme capable of cleaving the N-terminal truncated proHp at the enzymatic cleavage site;
   wherein, upon introduction of the first nucleic acid sequence and the second nucleic acid sequence into a mammalian cell, and subsequent expression of the N-terminal truncated proHp and the enzyme in the cell, the enzyme is capable of cleaving the N-terminal truncated proHp at the internal enzymatic cleavage site, thereby releasing the haptoglobin beta chain, or haemoglobin-binding fragment thereof, from the N-terminal truncated proHp.

The expression system described herein advantageously utilises mammalian cells as they are able to produce recombinant proteins that are likely to remain biological active, e.g., by facilitating proper folding of the protein and post-translational modifications required to preserve function in the expressed protein(s). As noted elsewhere herein, the inventors have unexpectedly found that their expression system advantageously results in stable transfection and expression of a functional haptoglobin β-chain and can therefore be distinguished from existing expression systems that achieve, at best, transient transfection and generally fail to generate functional protein. Thus, the expression system described herein is capable of stable transfection and expression of a functional recombinant haptoglobin beta chain, or a haemoglobin-binding fragment thereof, in a mammalian cell. Suitable methods of preparing recombinant proteins will be familiar to persons skilled in the art, illustrative examples of which include the introduction of one or more nucleic acid molecules comprising nucleic acid sequence/s encoding the desired recombinant protein, as herein described, into a suitable host cell capable of expressing said nucleic acid sequence, incubating said host cell under conditions suitable for the expression of said nucleic acid sequence, and recovering said recombinant protein.

Suitable methods for preparing a nucleic acid molecule encoding the recombinant protein will also be known to persons skilled in the art, based on knowledge of the genetic code, possibly including optimizing codons based on the nature of the host cell (e.g. human and non-human mammalian cells) to be used for expressing and/or secreting the recombinant fusion protein. Suitable mammalian cells for expression of recombinant proteins will also be known to persons skilled in the art, illustrative examples of which include chinese hamster ovary (CHO) cells and derivatives thereof (e.g., CHO-K1 and CHO pro-3), mouse myeloma cells (e.g., NSO and Sp2/0 cells), Human embryonic kidney cells (e.g., HEK 293). Protein expression in mammalian cells can also be achieved using viral-mediated transduction by such techniques as the BacMam system.10 This technology utilizes recombinant baculoviruses for simple transduction of mammalian cells, allowing for production of milligram quantities of protein for structural studies. 11 Other cell lines such as COS and Vero (both green African monkey kidney), HeLa (Human cervical cancer), and NSO (Mouse myeloma) have also been used for structural studies. Some of these cell lines such as NSO are more difficult to transfect. Transfection can be usually achieved using electroporation, and are only used in stable cell line production. Illustrative examples of suitable mammalian cells for expression of recombinant proteins, including those described herein, are described in Khan KH (2013, Adv. Pharm. Bull.; 3(2):257-263), such as U20S, A549, HT1080, CAD, P19, NIH 3T3, L929, N2a, human embryonic kidney 293 cells, HEK293T cells, Chinese hamster ovary cell lines, MCF-7, Y79, SO-Rb50, Hep G2, DUKX-X11, J558L and baby hamster kidney (BHK) cells.

Reference is also made to “Short Protocols in Molecular Biology, 5th Edition, 2 Volume Set: A Compendium of Methods from Current Protocols in Molecular Biology” (by Frederick M. Ausubel (author, editor), Roger Brent (editor), Robert E. Kingston (editor), David D. Moore (editor), J. G. Seidman (editor), John A. Smith (editor), Kevin Struhl (editor), J Wiley & Sons, London).

In an aspect of the invention, there is provided a mammalian cell comprising the first and second nucleic acid sequences according to the present invention, wherein the mammalian is capable of expressing of the N-terminal truncated proHp and the serine protease in the cell, the serine protease is capable of cleaving the N-terminal truncated proHp at the internal C1rLP cleavage site, thereby releasing the haptoglobin beta chain, or haemoglobin-binding fragment thereof, from the N-terminal truncated proHp of the present invention. Suitable mammalian cells are known to persons skilled in the art, illustrative examples of which include CHO, COS-7, Vero, NIH 3T3, L929, N2a, BHK, mouse ES cells, and human cells such as HeLa, HEK-293, HEK-293T, U20S, A549, HT1080, WI-38, MRC-5, Namalwa, HepG2 cells.

As used herein, the terms “encode,” “encoding” and the like refer to the capacity of a nucleic acid to provide for another nucleic acid or a polypeptide. For example, a nucleic acid sequence is said to “encode” a polypeptide if it can be transcribed and/or translated, typically in a host cell, to produce the polypeptide or if it can be processed into a form that can be transcribed and/or translated to produce the polypeptide. Such a nucleic acid sequence may include a coding sequence or both a coding sequence and a non-coding sequence. Thus, the terms “encode,” “encoding” and the like include an RNA product resulting from transcription of a DNA molecule, a protein resulting from translation of an RNA molecule, a protein resulting from transcription of a DNA molecule to form an RNA product and the subsequent translation of the RNA product, or a protein resulting from transcription of a DNA molecule to provide an RNA product, processing of the RNA product to provide a processed RNA product (e.g., mRNA) and the subsequent translation of the processed RNA product. In some embodiments, the nucleic acid sequence encoding the peptide sequences, as herein described, or the fusion proteins, as herein described, are codon-optimised for expression in a suitable host cell. For example, where the recombinant protein is to be used for treating or preventing a condition associated with cell-free haemoglobin (Hb) in a human subject, the nucleic acid sequences can be human codon-optimised. Suitable methods for codon optimisation would be known to persons skilled in the art, such as using the “Reverse Translation” option of ‘Gene Design” tool located in “Software Tools” on the John Hopkins University Build a Genome website.

As noted elsewhere herein, sequences can be linked to one another within the recombinant protein by any means known to persons skilled in the art. The terms “link” and “linked” include direct linkage of two sequences (e.g. peptide sequences) via a peptide bond; that is, the C-terminus of one sequence is covalently bound via a peptide bond to the N-terminal of another sequence. The terms “link” and “linked” also include within their meaning the linkage of two sequences (e.g. peptide sequences) via an interposed linker element.

Isolation and cloning of the nucleic acid sequences can be achieved using standard techniques (see, e.g., Ausubel et al., ibid.). For example, any desired nucleic acid sequence can be obtained directly from the virus by extracting RNA by standard techniques and then synthesizing cDNA from the RNA template (e.g., by RT-PCR). The nucleic acid sequence is then inserted directly or after one or more subcloning steps into a suitable expression vector. Persons skilled in the art will understand that the precise vector used is not critical. Illustrative examples of suitable vectors include plasmids, phagemids, cosmids, bacteriophage, baculoviruses, retroviruses or DNA viruses. The desired recombinant protein(s) can then be expressed and purified as described in more detail below. Alternatively, the nucleic acid sequence can be further engineered to introduce one or more mutations, such as those described above, by standard in vitro site-directed mutagenesis techniques known to persons skilled in the art. Mutations can be introduced by deletion, insertion, substitution, inversion, or a combination thereof, of one or more of the appropriate nucleotides making up the coding sequence. This can be achieved, for example, by PCR based techniques for which primers are designed that incorporate one or more nucleotide mismatches, insertions or deletions. The presence of the mutation can be verified by a number of standard techniques, for example by restriction analysis or by DNA sequencing. Methods for making recombinant proteins are well known to those skilled in the art. DNA sequences encoding recombinant protein can be inserted into a suitable expression vector, selection of which, would be known to a person skilled in the art. Suitable examples of expressions vectors include, and are not limited to the following. If the recombinant protein(s) is to be expressed in mammalian cells such as CHO, COS, and NIH3T3 cells, the expression vector includes a promoter necessary for expression in these cells, for example, an SV40 promoter (Mulligan et al., Nature, 277:108 (1979)) (e.g., early simian virus 40 promoter), MMLV-LTR promoter, EF1a promoter (Mizushima et al., Nucleic Acids Res., 18:5322 (1990)), or CMV promoter (e.g., human cytomegalovirus immediate early promoter). The recombinant expression vectors may carry additional sequences, such as sequences that regulate replication of the vector in host cells (e.g., origins of replication) and selectable marker genes. The selectable marker gene facilitates selection of host cells into which the vector has been introduced (see e.g., U.S. Pat. Nos. 4,399,216, 4,634,665 and 5,179,017). For example, typically the selectable marker gene confers resistance to drugs, such as G418, hygromycin, or methotrexate, on a host cell into which the vector has been introduced. Examples of vectors with selectable markers include pMAM, pDR2, pBK-RSV, pBK-CMV, pOPRSV, and pOP13.

It will be understood that the expression vector may further include regulatory elements, such as transcriptional elements, required for efficient transcription of the DNA sequence encoding the coat or fusion protein. Illustrative examples of suitable regulatory elements that can be incorporated into the vector include promoters, enhancers, terminators, and polyadenylation signals (e.g., derived from SV40, CMV, adenovirus and the like, such as a CMV enhancer/AdMLP promoter regulatory element or an SV40 enhancer/AdMLP promoter regulatory element, selected according to the host cell) to drive high levels of transcription of the nucleic acids.

In an embodiment, the nucleic acids, as herein described, are incorporated into a nucleic acid cassette, also referred to herein as an expression cassette. A nucleic acid cassette or expression cassette is intended to mean a nucleic acid sequence designed to introduce a nucleic acid sequence, typically a heterologous nucleic acid sequence (e.g., the nucleic acid construct as described herein) into vector. The expression cassette may include a terminal restriction enzyme linker (i.e., Restriction Enzyme recognition nucleotides) at each end of the sequence of the cassette to facilitate insertion of the nucleic acid sequence or sequences of interest. The terminal restriction enzyme linkers at each end may be the same or different terminal restriction enzyme linkers. In some embodiments, the terminal restriction enzyme linkers may include rare restriction enzyme recognition/cleavage sequences, such that unintended digestion of the nucleic acid or the alphavirus genome into which the cassette is to be introduced does not occur. Suitable terminal restriction enzyme linkers would be known to persons skilled in the art. In an embodiment, the Restriction Enzyme recognition nucleotides for Pac I (TTAATTAA) is added to the 5′ end of each expression cassette and the Restriction Enzyme recognition nucleotides for Sbf I (CCTGCAGG) is added to the 3′ end of each expression cassette.

In an embodiment, the transcriptional and translational regulatory control sequences include a promoter sequence, a 5′ non-coding region, a cis-regulatory region such as a functional binding site for transcriptional regulatory protein or translational regulatory protein, an upstream open reading frame, Internal Ribosome Entry Site (IRES), transcriptional start site, translational start site, and/or nucleotide sequence which encodes a leader sequence, termination codon, translational stop site and a 3′ non-translated region.

In an embodiment, the first nucleic acid and second nucleic acid are cloned into the same expression cassette and are under the control of separate promoters. In another embodiment, the first nucleic acid and second nucleic acid are cloned into the same expression cassette and are under the control of the same promoter. In this context, a single promoter will drive the expression of two open reading frames. In another embodiment, the first nucleic acid and second nucleic acid are cloned into separate expression cassettes. It is to be understood that the expression system described here may, in some contexts, advantageously comprises each of the first and second nucleic acid sequences in separate expression vectors. Thus, in an embodiment, the expression system comprises (i) a first expression vector comprising the first nucleic acid sequence, as described herein, and (ii) a second expression vector comprising the second nucleic acid sequence, as described herein.

Expression cassettes contemplated herein may also comprise one or more selectable marker sequences suitable for use in the identification of host cells which have or have not been infected transformed or transfected with the expression cassette. Markers include, for example, genes encoding proteins which increase or decrease either resistance or sensitivity to antibiotics or other compounds, genes which encode enzymes whose activities are detectable by standard assays known in the art (e.g., p-galactosidase, luciferase), and genes which visibly affect the phenotype of transformed or transfected cells, hosts, colonies or plaques (e.g., various fluorescent proteins such as green fluorescent protein, GFP) carrying the expression cassette.

The present disclosure also extends to a host cell comprising the polynucleotide composition described herein.

As used herein, a host cell is understood to mean a cell comprising the polynucleotide composition described herein. The host cell can be a bacterial cell, a yeast cell, insect or a mammalian cell line. In a preferred embodiment, the host cell is an internal cell of the subject to which the polynucleotide composition described herein will be administered.

The host cell may be transfected and/or infected by a vector or progeny thereof such that it may express the polynucleotide composition described herein and produce the recombinant protein, as herein described.

Suitable host cell lines are known to those of skill in the art and are commercially available, for example, through established cell culture collections. Such cells may then be used to produce recombinant proHp, or for other uses as may be required. An exemplary method may comprise culturing a cell comprising the polynucleotide composition (e.g., optionally under the control of an expression sequence) under cell culture conditions that allow for the optimal production of the recombinant protein which may then be isolated from the cell or the cell culture medium using standard techniques known to persons skilled in the art. The present disclosure also extends to recombinant proteins isolated from the cultured mammalian cells modified to express the N-terminal truncated proHp as described herein, including recombinant haptoglobin beta chains and haemoglobin-binding fragments thereof, as well as any of the one or more functional moieties, as described elsewhere herein.

The expression system according to the present invention comprises a first nucleic acid sequence encoding an N-terminal truncated proHp, comprising at least 14 contiguous C-terminal amino acid residues of a haptoglobin alpha chain and a haptoglobin beta chain, or a haemoglobin-binding fragment thereof. The haemoglobin-binding fragment of the haptoglobin beta chain can be any suitable length, provided that the fragment retains the ability to form a complex with cell-free Hb and thereby neutralise its biological activity. The N-terminal truncated proHp further comprises an internal C1r-like protein (C1rLP) cleavage site between the at least 14 contiguous C-terminal amino acid residues of a haptoglobin alpha chain and the haptoglobin beta chain, or haemoglobin-binding fragment thereof. In preferred embodiments, the at least 14 contiguous C-terminal amino acid residues of a haptoglobin alpha chain comprises at least a cysteine residue. In an embodiment, a cysteine residue is located at the 14aa position of the at least 14 contiguous C-terminal amino acid residues of a haptoglobin alpha chain. Advantageously, the cysteine residue of the at least 14 contiguous C-terminal amino acid residues of the haptoglobin alpha chain may form a disulphide bond with the otherwise free cysteine residue of the beta chain, which may assist in the expression and/or purification of the recombinant haptoglobin beta chain, or a haemoglobin-binding fragment thereof.

In some embodiments, the expression of the N-terminal truncated proHp in the mammalian cell may be driven by a first mammalian regulatory sequence operably linked to the first nucleic acid sequence, and the expression of the serine protease in the mammalian cell may be driven by a second mammalian regulatory sequence operably linked to the second nucleic acid sequence. The first mammalian regulatory sequence may be the same or different to the second mammalian regulatory sequence. In an embodiment, the first mammalian regulatory sequence is different to the second mammalian regulatory sequence.

The present disclosure also extends to expression vectors for producing a recombinant haptoglobin beta chain, or a haemoglobin-binding fragment thereof, in a mammalian cell, as herein described. The vector may comprise the first nucleic acid sequence described herein and the second nucleic acid sequence described herein. The first nucleic acid and the second nucleic acid may be operably linked to the same or different mammalian regulatory sequence. Thus, in some embodiments, the first nucleic acid sequence and the second nucleic acid sequence may be operably linked to a common mammalian regulatory sequence. In other embodiments, the first nucleic acid sequence is operably linked to a first mammalian regulatory sequence and the second nucleic acid sequence is operably linked to a second mammalian regulatory sequence, and wherein the first mammalian regulatory sequence is different to the second mammalian regulatory sequence. As noted elsewhere herein, the present disclosure also extends to an expression system comprising (i) a first expression vector comprising the first nucleic acid sequence, as described herein, and (ii) a second vector comprising the second nucleic acid sequence, as described herein. The first nucleic acid sequence and the second nucleic acid sequence may each be operably linked to regulatory sequences, preferably to a mammalian regulatory sequence, as described herein.

The present disclosure also extends to methods of producing a recombinant haptoglobin beta chain, or a haemoglobin-binding fragment thereof, the method comprising introducing into a mammalian cell the expression system as herein described or the expression vector(s) as herein described. Suitable methods of introducing the expression system or the expression vector(s) into a mammalian cell will be familiar to persons skilled in the art. For example, biological (e.g., virus-mediated), chemical (e.g., cationic polymer, calcium phosphate, cationic lipid or cationic amino acid) or physical (e.g., direct injection, biolistic particle delivery, electroporation, laser-irradiation, sonoporation or magnetic nanoparticle) transfection methods may be employed. In an embodiment, introducing the expression system or the expression vector(s) into a mammalian cell is achieved using a cationic, lipid-based transfection reagent.

When the expression system or vector(s), as herein described, is introduced into a mammalian cell, the N-terminal truncated proHp and serine protease are expressed within the cell when the cell is cultured under suitable conditions. Without being limited by theory or a particular mode of application, it is understood that, upon expression, the expressed serine protease cleaves the expressed N-terminal truncated proHp at the internal C1rLP cleavage site, releasing the haptoglobin beta chain, or haemoglobin-binding fragment thereof, from the N-terminal truncated proHp. The cell will suitably be cultured under conditions and for a period of time sufficient to allow production of the recombinant haptoglobin beta chain or haemoglobin-binding fragment thereof. Suitable culture conditions and culture media, including commercially available cell culture media, will be familiar to persons skilled in the art, illustrative examples of which are described, for example, in Laurenti and Ooi (2013; 998:10.1007/978-1-62703-351-0_2; Methods in molecular biology (Clifton, N.J.) and Kaufman RJ (2000, Mol. Biotechnol.; 16:151-160). It is to be noted that the culture conditions and the time sufficient to allow for suitable expression of the recombinant proteins in the mammalian cell carrying the expression system disclosed herein may depend on the type of mammalian cell(s) that is/are being employed, noting that the kinetics of recombinant protein expression may vary between mammalian cell types. In any event, the culture conditions and culture times may be optimised by routine experimentation.

Non-limiting examples of suitable mammalian cells are described elsewhere herein and include human, bovine, ovine, equine, goat, rabbit, guinea pig, rat, hamster or mouse cells, HEK 293 (human embryonic kidney), CHO (Chinese hamster ovary) and mouse myeloma cells. Other illustrative examples of suitable mammalian cells include HeLa, HEK293T, U2OS, A549, HT1080, CAD, P19, NIH 3T3, L929, N2a, HEK 293, CHO, MCF-7, Y79, SO-Rb50, Hep G2, DUKX-X11, J558L and BHK cells. In an embodiment, the mammalian cell is a human cell. In another embodiment, the mammalian cell is a human embryonic cell, preferably a human embryonic kidney cell (e.g., HEK 293).

The present invention further provides a mammalian cell modified to carry the expression system as herein described.

The recombinant haptoglobin beta chain or haemoglobin-binding fragment thereof may be isolated and purified using any suitable method known in the art. In some examples, purification is performed by chromatography, such as tandem chromatography, as exemplified in the Examples.

### Enzymes Encoded by the Second Nucleic Acid Sequence

As noted elsewhere herein, the expression system disclosed herein comprises a second nucleic acid sequence encoding an enzyme that is capable of cleaving the N-terminal truncated proHp, encoded by the first nucleic acid sequence, at the enzymatic cleavage site described herein. Thus, it will be understood that the choice of enzyme encoded by the second nucleic acid sequence will depend on the internal enzymatic cleavage site between the at least 14 contiguous C-terminal amino acid residues of a haptoglobin alpha chain and the haptoglobin beta chain, or haemoglobin-binding fragment thereof, of the N-terminal truncated proHp; that is, the enzyme encoded by the second nucleic acid sequence will be compatible with the internal enzymatic cleavage site, such that the enzyme is suitably capable of cleaving the N-terminal truncated proHp at the enzymatic cleavage site when the first nucleic acid sequence and the second nucleic acid sequence are expressed in a mammalian cell. Suitable internal enzymatic cleavage sites will be familiar to persons skilled in the art, illustrative examples of which are described elsewhere herein, such as a furin cleavage site, a non-native serine protease cleavage site, a cysteine protease cleavage site, an aspartic protease cleavage site, a metalloprotease cleavage site, and a threonine protease cleavage site. In an embodiment, the enzyme encoded by the second nucleic acid sequence of the expression system described herein is selected from the group consisting of furin, a serine protease, a cysteine protease, an aspartic protease, a metalloprotease, and a threonine protease. In an embodiment, the enzyme encoded by the second nucleic acid sequence of the expression system described herein is a serine protease.

Suitable serine proteases will be familiar to persons skilled in the art, illustrative examples of which are described, for example, in Di Cera (IUBMB Life, 2009; 61(5):510-515). In an embodiment, the serine protease is C1r-like serine protease, C1rLP, or a functional variant thereof. The term “functional variant”, when used in relation to C1rLP, is to be understood to include serine proteases having an amino acid sequence that differs from its natural counterpart by one or more amino acid substitutions, deletions and/or insertions, including conservative or non-conservative amino acid substitutions, where such differences do not substantially alter the ability of the variant to cleaving the N-terminal truncated proHp at the internal C1rLP cleavage site. The functional variant may be naturally-occurring, recombinant or synthetic (e.g., produced by chemical synthesis) using methods known to persons skilled in the art. Functional variant of C1rLP extend to naturally-occurring isoforms, examples of which will be known to persons skilled in the art, such as C1rLP isoform 1 (e.g., GenBank Accession No. NP_057630; SEQ ID NO:4), C1rLP isoform 2 (e.g., GenBank Accession No. NP_001284569; SEQ ID NO:5), C1rLP isoform 3 (e.g., GenBank Accession No. NP_001284571; SEQ ID NO:6), and C1rLP isoform 4 (e.g., GenBank Accession No. NP_001284572; SEQ ID NO:7). In an embodiment, the C1rLP serine protease comprises, consists or consists essentially of the amino acid sequence of any one of SEQ ID NOs:4-7. In an embodiment, the serine protease or functional variant thereof comprises, consists, or consists essentially of the amino acid sequence of SEQ ID NO:4. In another embodiment, the serine protease or functional variant thereof comprises, consists, or consists essentially of the amino acid sequence of SEQ ID NO:5. In another embodiment, the serine protease or functional variant thereof comprises, consists, or consists essentially of the amino acid sequence of SEQ ID NO:6. In another embodiment, the serine protease or functional variant thereof comprises, consists, or consists essentially of the amino acid sequence of SEQ ID NO:7. In an embodiment, the serine protease or functional variant thereof is a C1rLP comprising, consisting or consisting essentially of an amino acid sequence having at least 80%, preferably at least 85%, preferably at least 86%, preferably at least 87%, preferably at least 88%, preferably at least 89%, preferably at least 90%, preferably at least 91%, preferably at least 92%, preferably at least 93%, preferably at least 94%, preferably at least 95%, preferably at least 96%, preferably at least 97%, preferably at least 98%, preferably at least 99% or preferably 100% sequence identity to any one of SEQ ID NOs:4-7, for example, after optimal alignment or best fit analysis. In an embodiment, the serine protease or functional variant thereof is a C1rLP comprising, consisting or consisting essentially of an amino acid sequence having at least 80%, preferably at least 85%, preferably at least 86%, preferably at least 87%, preferably at least 88%, preferably at least 89%, preferably at least 90%, preferably at least 91%, preferably at least 92%, preferably at least 93%, preferably at least 94%, preferably at least 95%, preferably at least 96%, preferably at least 97%, preferably at least 98%, preferably at least 99% or preferably 100% sequence identity to any one of SEQ ID NO:4, for example, after optimal alignment or best fit analysis. In an embodiment, the serine protease or functional variant thereof is a C1rLP comprising, consisting or consisting essentially of an amino acid sequence having at least 80%, preferably at least 85%, preferably at least 86%, preferably at least 87%, preferably at least 88%, preferably at least 89%, preferably at least 90%, preferably at least 91%, preferably at least 92%, preferably at least 93%, preferably at least 94%, preferably at least 95%, preferably at least 96%, preferably at least 97%, preferably at least 98%, preferably at least 99% or preferably 100% sequence identity to any one of SEQ ID NO:5, for example, after optimal alignment or best fit analysis. In an embodiment, the serine protease or functional variant thereof is a C1rLP comprising, consisting or consisting essentially of an amino acid sequence having at least 80%, preferably at least 85%, preferably at least 86%, preferably at least 87%, preferably at least 88%, preferably at least 89%, preferably at least 90%, preferably at least 91%, preferably at least 92%, preferably at least 93%, preferably at least 94%, preferably at least 95%, preferably at least 96%, preferably at least 97%, preferably at least 98%, preferably at least 99% or preferably 100% sequence identity to any one of SEQ ID NO:6, for example, after optimal alignment or best fit analysis. In an embodiment, the serine protease or functional variant thereof is a C1rLP comprising, consisting or consisting essentially of an amino acid sequence having at least 80%, preferably at least 85%, preferably at least 86%, preferably at least 87%, preferably at least 88%, preferably at least 89%, preferably at least 90%, preferably at least 91%, preferably at least 92%, preferably at least 93%, preferably at least 94%, preferably at least 95%, preferably at least 96%, preferably at least 97%, preferably at least 98%, preferably at least 99% or preferably 100% sequence identity to any one of SEQ ID NO:7, for example, after optimal alignment or best fit analysis.

### Pharmaceutical Compositions and Uses Thereof

The present disclosure also extends to pharmaceutical compositions comprising a therapeutically effective amount of a recombinant haptoglobin beta chain, or haemoglobin-binding fragment thereof, prepared according to the methods described herein, optionally comprising a pharmaceutically acceptable carrier.

The present disclosure also extends to a recombinant haemoglobin-binding molecule comprising (i) a haptoglobin beta chain, or a haemoglobin-binding fragment thereof, and (ii) an N-terminal truncated haptoglobin alpha chain, wherein the N-terminal truncated haptoglobin alpha chain comprises at least 14 contiguous C-terminal amino acid residues of the haptoglobin alpha chain, wherein the at least 14 contiguous C-terminal amino acid residues of the haptoglobin alpha chain is non-contiguous to the haptoglobin beta chain, or the haemoglobin-binding fragment thereof, and wherein the N-terminal truncated haptoglobin alpha chain is attached to the haptoglobin beta chain, or the haemoglobin-binding fragment thereof. By “non-contiguous” is meant that the recombinant haemoglobin-binding molecule does not comprise an amino acid sequence that corresponds to the amino acid sequence bridging the alpha and beta chains of a native proHp.

In an embodiment, the N-terminal truncated haptoglobin alpha chain of the recombinant haemoglobin-binding molecule is attached to the haptoglobin beta chain, or the haemoglobin-binding fragment thereof, as described herein, by a disulphide bond formed between a cysteine residue in the haptoglobin beta chain, or the haemoglobin-binding fragment thereof, and a cysteine residue in the at least 14 contiguous C-terminal amino acid residues of the haptoglobin alpha chain. In an embodiment, the haptoglobin beta chain, or the haemoglobin-binding fragment thereof, comprises an amino acid sequence having at least 80% sequence identity to amino acid residues 162 to 406 of SEQ ID NO:1.

In another embodiment, the haemoglobin-binding molecule further comprises an additional functional moiety. In an embodiment, the additional functional moiety is attached to the N-terminal truncated haptoglobin alpha chain. Suitable functional moieties will be familiar to persons skilled in the art, illustrative examples of which are described elsewhere herein. In an embodiment, the additional functional moiety is selected from the group consisting of a heme-binding moiety, an Fc domain of an immunoglobulin, or an FcRn-binding fragment thereof and albumin. In an embodiment, the additional functional moiety is a heme-binding moiety. In a preferred embodiment, the heme-binding moiety is hemopexin, or a heme-binding fragment thereof.

In an embodiment, the composition comprises from about 2 μM to about 20 mM recombinant haptoglobin beta chain, or haemoglobin-binding fragment thereof. In an embodiment, the composition comprises from about 2 μM to about 5 mM recombinant haptoglobin beta chain, or haemoglobin-binding fragment thereof. In an embodiment, the composition comprises from about 100 μM to about 5 mM recombinant haptoglobin beta chain, or haemoglobin-binding fragment thereof, or a functional analogue thereof. In an embodiment, the composition comprises from about 2 μM to about 300 μM recombinant haptoglobin beta chain, or haemoglobin-binding fragment thereof. In an embodiment, the composition comprises from about 5 μM to about 50 μM recombinant haptoglobin beta chain, or haemoglobin-binding fragment thereof. In an embodiment, the composition comprises from about 10 μM to about 30 μM recombinant haptoglobin beta chain, or haemoglobin-binding fragment thereof.

The pharmaceutical compositions disclosed herein may be formulated for any suitable route of administration, illustrative example of which include intravascular, intrathecal, intracranial and intracerebroventricular administration.

In an embodiment, the pharmaceutical compositions disclosed herein are formulated for intrathecal administration. Suitable intrathecal delivery systems will be familiar to persons skilled in the art, illustrative examples of which are described by Kilburn et al. (2013, Intrathecal Administration. In: Rudek M., Chau C., Figg W., McLeod H. (eds) Handbook of Anticancer Pharmacokinetics and Pharmacodynamics. Cancer Drug Discovery and Development. Springer, New York, NY), the contents of which are incorporated herein by reference in their entirety.

In another embodiment, the pharmaceutical compositions disclosed herein are formulated for intracranial administration. Suitable intracranial delivery systems will be familiar to persons skilled in the art, illustrative examples of which are described by Upadhyay et al. (2014, PNAS, 111(45):16071-16076), the contents of which are incorporated herein by reference in their entirety.

In another embodiment, the pharmaceutical compositions disclosed herein are formulated for intracerebroventricular administration. Suitable intracerebroventricular delivery systems will be familiar to persons skilled in the art, illustrative examples of which are described by Cook et al. (2009, Pharmacotherapy. 29(7):832-845), the contents of which are incorporated herein by reference in their entirety.

The present disclosure also extends to unit dosage forms of the pharmaceutical compositions described herein. Suitable pharmaceutical compositions and unit dosage forms thereof may comprise conventional ingredients in conventional proportions, with or without additional active compounds or principles, and such unit dosage forms may contain any suitable effective amount of the active ingredient commensurate with the intended daily dosage range to be employed.

The recombinant haptoglobin beta chain, or haemoglobin-binding fragment thereof, as herein described, and pharmaceutical compositions comprising the same, may be used for the treatment of conditions associated with cell-free haemoglobin (Hb), including, but not limited to haemorrhagic stroke, Sickle cell disease, erythrolysis and a haemoglobinopathy.

Haemorrhagic stroke is typically characterised by a ruptured blood vessel in the brain causing localized bleeding (haemorrhage). The location of the bleed can vary and the type of haemorrhagic stroke is characterised by this location. Examples of haemorrhagic stroke include i) intracerebral haemorrhage which involves a blood vessel in the brain bursting; ii) intraventricular haemorrhage which is bleeding into the brains ventricular system; and iii) subarachnoid haemorrhage (SAH) which involves bleeding in the space between the brain and the tissue covering the brain known as the subarachnoid space. Most often SAH is caused by a burst aneurysm, referred to as aneurysmal subarachnoid haemorrhage (aSAH). Other causes of SAH include head injury, bleeding disorders and the use of blood thinners. Haemorrhagic stroke is made up of a range of pathologies with different natural courses, assessment, and management, as will be familiar to persons skilled in the art. It is generally categorized as primary or secondary, depending on aetiology.

Methods of diagnosing a haemorrhagic stroke, and in particular SAH, in a subject will be familiar to persons skilled in the art, illustrative examples of which include cerebral angiography, computerised tomography (CT) and spectrophotometric analysis of oxyHb and bilirubin in the subject's CSF (see, for example, Cruickshank AM., 2001, ACP Best Practice No 166, J. Clin. Path., 54(11):827-830).

It will be understood by persons skilled in the art that the haemorrhagic stroke can be a spontaneous haemorrhage (e.g., as a result of a ruptured aneurysm) or a traumatic haemorrhage (e.g., as a result of a trauma to the head). In an embodiment, the haemorrhagic stroke is a spontaneous haemorrhage, also known as a non-traumatic haemorrhage. In an embodiment, the haemorrhagic stroke is a traumatic haemorrhage.

In some embodiments, the haemorrhagic stroke is an intraventricular haemorrhage or a subarachnoid haemorrhage. The subarachnoid haemorrhage may be an aneurysmal subarachnoid haemorrhage (aSAH).

Haemoglobinopathies are a group of hereditary disorders characterised by genetic abnormalities that affect haemoglobin. These abnormalities are caused by mutations and/or deletions in the α- or β-globin genes. Examples of haemoglobinopathies include sickle cell disease, which is associated with structural abnormalities in haemoglobin, and thalassaemia, which is associated with insufficient haemoglobin production. Other haemoglobinopathies associated with erythrolysis and release of cell-free Hb will be known to those skilled in the art. There are many forms of thalassaemia, which can be broadly categorised as α- and β-thalassaemia, depending on the whether they are associated with an α- or β-globin chain synthesis defect. Each haemoglobinopathy disorder is associated with unique and highly variable pathologies with different natural courses, assessment, and management, as will be familiar to persons skilled in the art. Methods of diagnosing a haemoglobinopathy in a subject will be familiar to persons skilled in the art. For example, diagnosis may involve a red blood cell count with erythrocyte indices, and a hemoglobin test, such as hemoglobin electrophoresis and/or chromatography, followed by DNA test if indicated.

In some embodiments, the haemoglobinopathy is sickle cell disease. In other embodiments, the haemoglobinopathy is a thalassemia. The thalassemia may be a-thalassemia or p-thalassemia.

A skilled person will appreciate that the recombinant haptoglobin beta chain or haemoglobin-binding fragment thereof, or pharmaceutical composition as disclosed herein are suitable for use in the treatment or prevention of any disease, condition or disorder associated with cell-free Hb. Such diseases, conditions and disorders will be known to those skilled in the art.

The term “therapeutically effective amount”, as used herein, means the amount or concentration of recombinant haptoglobin beta chain, or haemoglobin-binding fragment thereof, is sufficient to allow the Hp to bind to, and form a complex with, cell-free Hb present and thereby neutralise the otherwise adverse biological effect of the cell-free Hb. It would be understood by persons skilled in the art that the therapeutically effective amount of peptide may vary depending upon several factors, illustrative examples of which include whether the recombinant haptoglobin beta chain, or haemoglobin-binding fragment thereof is to be administered directly to the subject (e.g., intravascularly, intrathecally, intracranially or intracerebroventricularly) or as a pharmaceutical compositions, the health and physical condition of the subject to be treated, the taxonomic group of subject to be treated, the severity of the condition (e.g., the extent of bleeding), the route of administration, the concentration and/or amount of cell-free Hb to be neutralised and combinations of any of the foregoing.

The terms “treating”, “treatment”, “treat” and the like, are used interchangeably herein to mean relieving, minimising, reducing, alleviating, ameliorating or otherwise inhibiting one or more symptoms associated with a condition associated with cell-free Hb. The terms “treating”, “treatment” and the like are also used interchangeably herein to mean preventing conditions associated with cell-free Hb from occurring or delaying the onset or subsequent progression of a conditions associated with cell-free Hb in a subject that may be predisposed to, or at risk of, developing a condition associated with cell-free Hb, but has not yet been diagnosed as having it. In that context, the terms “treating”, “treatment” and the like are used interchangeably with terms such as “prophylaxis”, “prophylactic” and “preventative”. It is to be understood, however, that the methods disclosed herein need not completely prevent a condition associated with cell-free Hb from occurring in the subject to be treated. It may be sufficient that the methods disclosed herein merely relieve, reduce, alleviate, ameliorate or otherwise inhibit a condition associated with cell-free Hb in the subject to the extent that there are fewer symptoms and/or less severe adverse symptoms than would otherwise have been observed in the absence of treatment. Thus, the methods described herein may reduce the number and/or severity of conditions associated with cell-free Hb.

The therapeutically effective amount of recombinant haptoglobin beta chain, or haemoglobin-binding fragment thereof, will typically fall within a relatively broad range that can be determined by persons skilled in the art. Illustrative examples of a suitable therapeutically effective amounts of recombinant haptoglobin beta chain, or haemoglobin-binding fragment thereof, include from about 2 μM to about 20 mM, preferably from about 2 μM to about 5 mM, preferably from about 100 μM to about 5 mM, preferably from about 2 μM to about 300 μM, preferably from about 5 μM to about 100 μM, preferably from about 5 μM to about 50 μM, or more preferably from about 10 μM to about 30 μM.

In an embodiment, the therapeutically effective amount of recombinant haptoglobin beta chain, or haemoglobin-binding fragment thereof, is from about 2 μM to about 20 mM. In an embodiment, the therapeutically effective amount of recombinant haptoglobin beta chain, or haemoglobin-binding fragment thereof, is from about 2 μM to about 5 mM. In an embodiment, the therapeutically effective amount of recombinant haptoglobin beta chain, or haemoglobin-binding fragment thereof, is from about 100 μM to about 5 mM. In an embodiment, the therapeutically effective amount of recombinant haptoglobin beta chain, or haemoglobin-binding fragment thereof, is from about 2 μM to about 300 μM. In an embodiment, the therapeutically effective amount of recombinant haptoglobin beta chain, or haemoglobin-binding fragment thereof, is from about 5 μM to about 50 μM. In an embodiment, the therapeutically effective amount of recombinant haptoglobin beta chain, or haemoglobin-binding fragment thereof, is from about 10 μM to about 30 μM.

In an embodiment, the therapeutically effective amount of recombinant haptoglobin beta chain, or haemoglobin-binding fragment thereof, is at least an equimolar amount to the concentration of cell-free Hb to be neutralised. In the case of haemorrhagic stroke, the therapeutically effective amount of recombinant haptoglobin beta chain, or haemoglobin-binding fragment thereof, is an amount sufficient to complex from about 3 μM to about 300 μM cell-free Hb in CSF. Suitable methods of measuring the concentration of cell-free Hb in CSF will be known to persons skilled in the art, illustrative examples of which are described in Cruickshank AM., 2001, ACP Best Practice No 166, J. Clin. Path., 54(11):827-830) and Hugelshofer M. et al., 2018. World Neurosurg.;120:e660-e666), the contents of which are incorporated herein by reference in their entirety.

Dosages of recombinant haptoglobin beta chain, or haemoglobin-binding fragment thereof, may also be adjusted to provide the optimum therapeutic response. For example, several divided doses may be administered daily, weekly, or other suitable time intervals, or the dosages may be proportionally reduced as indicated by the exigencies of the situation.

In an embodiment, the dosage of recombinant haptoglobin beta chain, or haemoglobin-binding fragment thereof, is sufficient to substantially neutralise the cell-free Hb. By “substantially neutralise” is meant a reduction in the amount of cell-free Hb, as represented subjectively or qualitatively as a percentage reduction by at least 10%, preferably from about 10% to about 20%, preferably from about 15% to about 25%, preferably from about 20% to about 30%, preferably from about 25% to about 35%, preferably from about 30% to about 40%, preferably from about 35% to about 45%, preferably from about 40% to about 50%, preferably from about 45% to about 55%, preferably from about 50% to about 60%, preferably from about 55% to about 65%, preferably from about 60% to about 70%, preferably from about 65% to about 75%, preferably from about 70% to about 80%, preferably from about 75% to about 85%, preferably from about 80% to about 90%, preferably from about 85% to about 95%, or most preferably from about 90% to 100% compared to the biological effect of cell-free Hb in the absence of therapeutic recombinant haptoglobin beta chain, or haemoglobin-binding fragment thereof, as described herein, including by at least 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100%. Methods by which the amount of cell-free Hb can be measured or determined (qualitatively or quantitatively) will be familiar to persons skilled in the art.

The present invention also provides a method of treating or preventing a condition associated with cell-free haemoglobin (Hb) in a subject, the method comprising administering to a subject in need thereof a therapeutically effective amount of the recombinant haptoglobin beta chain, or haemoglobin-binding fragment thereof, prepared according to the methods described herein, for a period of time sufficient to allow the haptoglobin beta chain, or haemoglobin-binding fragment thereof, to form a complex with, and thereby neutralise, the cell-free Hb.

The haptoglobin beta chain, or haemoglobin-binding fragment thereof, and pharmaceutical compositions described herein may be administered to a subject by any suitable method known in the art. For example, the haptoglobin beta chain, or haemoglobin-binding fragment thereof, and pharmaceutical compositions described herein may be administered by oral, injectable, parenteral, subcutaneous, intravenous, intravitreal or intramuscular delivery. In some embodiments, the haptoglobin beta chain, or haemoglobin-binding fragment thereof, and pharmaceutical compositions may also be formulated for sustained delivery.

In an embodiment, the method comprises intravascularly administering to the subject the therapeutically effective amount of the recombinant haptoglobin beta chain, or haemoglobin-binding fragment thereof.

In an embodiment, the method comprises intracranially administering to the subject the therapeutically effective amount of the recombinant haptoglobin beta chain, or haemoglobin-binding fragment thereof.

In an embodiment, the method comprises intrathecally administering to the subject the therapeutically effective amount of the recombinant haptoglobin beta chain, or haemoglobin-binding fragment thereof. In an embodiment, the method comprises intrathecally administering to the subject the therapeutically effective amount of the recombinant haptoglobin beta chain, or haemoglobin-binding fragment thereof into the spinal canal. In an embodiment, the method comprises intrathecally administering to the subject the therapeutically effective amount of the recombinant haptoglobin beta chain, or haemoglobin-binding fragment thereof into the subarachnoid space.

In an embodiment, the method comprises intracerebroventricularly administering to the subject the therapeutically effective amount of the recombinant haptoglobin beta chain, or haemoglobin-binding fragment thereof.

The term “subject”, as used herein, refers to a mammalian subject for whom treatment or prophylaxis is desired. Illustrative examples of suitable subjects include primates, especially humans, companion animals such as cats and dogs and the like, working animals such as horses, donkeys and the like, livestock animals such as sheep, cows, goats, pigs and the like, laboratory test animals such as rabbits, mice, rats, guinea pigs, hamsters and the like and captive wild animals such as those in zoos and wildlife parks, deer, dingoes and the like. In an embodiment, the subject is a human. In a further embodiment, the subject is a paediatric patient aged (i) from birth to about 2 years of age, (ii) from about 2 to about 12 years of age or (iii) from about 12 to about 21 years of age.

The present disclosure also extends to the use of a therapeutically effective amount of the recombinant haptoglobin beta chain, or haemoglobin-binding fragment thereof, prepared according to the methods described herein, in the manufacture of a medicament for treating or preventing a condition associated with cell-free haemoglobin (Hb) in a subject.

### Adjunct Therapy

Methods of treating or preventing conditions associated with cell-free Hb, as described herein, may suitably be performed together, either sequentially or in combination (e.g., at the same time), with one or more another treatment strategies designed to reduce, inhibit, prevent or otherwise alleviate the condition associated with cell-free Hb. In an embodiment, the methods described herein further comprise administering to the subject at least one additional therapeutic agent for treating or preventing a condition associated with erythrolysis and release of cell-free Hb. Suitable adjunct therapies and therapeutic agents for treating or preventing a condition associated with conditions associated with cell-free haemoglobin (Hb) will be familiar to persons skilled in the art, illustrative examples of which include:
(i) Coagulopathy correction—e.g., using vitamin K antagonists (VKAs), novel oral anticoagulants (NOAC, such as dabigatran, rivaroxaban, and apixaban), factor eight inhibitor bypass activity (FEIBA) and activated recombinant factor VII (rFVIIa), prothrombin complex concentrate, activated charcoal, antiplatelet therapy (APT), and aspirin monotherapy;
(ii) Lowering blood pressure—e.g., antihypertensive agents, illustrative examples of which include (i) diuretics, such as thiazides, including chlorthalidone, chlorthiazide, dichlorophenamide, hydroflumethiazide, indapamide, and hydrochlorothiazide; loop diuretics, such as bumetanide, ethacrynic acid, furosemide, and torsemide; potassium sparing agents, such as amiloride, and triamterene; and aldosterone antagonists, such as spironolactone, epirenone, and the like; (ii) beta-adrenergic blockers such as acebutolol, atenolol, betaxolol, bevantolol, bisoprolol, bopindolol, carteolol, carvedilol, celiprolol, esmolol, indenolol, metaprolol, nadolol, nebivolol, penbutolol, pindolol, propanolol, sotalol, tertatolol, tilisolol, and timolol, and the like; (iii) calcium channel blockers such as amlodipine, aranidipine, azelnidipine, barnidipine, benidipine, bepridil, cinaldipine, clevidipine, diltiazem, efonidipine, felodipine, gallopamil, isradipine, lacidipine, lemildipine, lercanidipine, nicardipine, nifedipine, nilvadipine, nimodepine, nisoldipine, nitrendipine, manidipine, pranidipine, and verapamil, and the like; (iv) angiotensin converting enzyme (ACE) inhibitors such as benazepril; captopril; cilazapril; delapril; enalapril; fosinopril; imidapril; losinopril; moexipril; quinapril; quinaprilat; ramipril; perindopril; perindropril; quanipril; spirapril; tenocapril; trandolapril, and zofenopril, and the like; (v) neutral endopeptidase inhibitors such as omapatrilat, cadoxatril and ecadotril, fosidotril, sampatrilat, AVE7688, ER4030, and the like; (vi) endothelin antagonists such as tezosentan, A308165, and YM62899, and the like; (vii) vasodilators such as hydralazine, clonidine, minoxidil, and nicotinyl alcohol, and the like; (viii) angiotensin II receptor antagonists such as candesartan, eprosartan, irbesartan, losartan, pratosartan, tasosartan, telmisartan, valsartan, and EXP-3137, F16828K, and RNH6270, and the like; (ix) α/β adrenergic blockers as nipradilol, arotinolol and amosulalol, and the like; (x) alpha 1 blockers, such as terazosin, urapidil, prazosin, bunazosin, trimazosin, doxazosin, naftopidil, indoramin, WHIP 164, and XENOIO, and the like; and (xi)-alpha 2 agonists such as lofexidine, tiamenidine, moxonidine, rilmenidine and guanobenz, and the like;
(ii-b) Vasodilators—e.g., hydralazine (apresoline), clonidine (catapres), minoxidil (loniten), nicotinyl alcohol (roniacol), sydnone and sodium nitroprusside;
(iii) Management of Seizures, Glucose and Temperature—e.g., antiepileptic drugs, insulin infusions to control blood glucose levels, maintenance of normo-thermia and therapeutic cooling;
(iv) Surgical treatment—e.g., hematoma evacuation (surgical clot removal), decompressive craniectomy (DC), minimally invasive surgery (MIS; such as needle aspiration of basal ganglia haemorrhages), MIS with recombinant tissue-type plasminogen activator (rtPA);
(v) Timing of Surgery—e.g., from 4 to 96 hours after symptom onset;
(vi) Thrombin Inhibition—e.g., hirudin, argatroban, serine protease inhibitors (e.g., nafamostat mesilate);
(vii) Prevention of Heme and Iron Toxicity—e.g., non-specific heme oxygenase (HO) inhibitors such as tin-mesoporphyrin, iron chelators such as deferoxamine;
(viii) PPARg antagonists and agonists—e.g., rosiglitazone, 15d-PGJ2 and pioglitazone;
(ix) Inhibition of microglial activation—e.g., tuftsin fragment 1-3 (a microglia/macrophage inhibitory factor) or minocycline (a tetracycline-class antibiotic);
(x) Upregulation of NF-Erythroid-2-Related Factor 2 (Nrf2);
(xi) Cyclo-Oxygenase (COX) Inhibition—e.g., celecoxib (a selective COX-2 inhibitor);
(xii) Matrix Metalloproteinases;
(xiii) TNF-α modulators—e.g., adenosine receptor agonists such as CGS 21680, TNF-α-specific antisense oligodeoxynucleotides such as ORF4-PE;
(xiv) Raising blood pressure—e.g., catecholamines; and
(xv) Inhibitors of TLR4 signalling—e.g., antibody Mts510 and TAK-242 (a cyclohexene derivative).

In an embodiment, the additional therapeutic agent is the one or more functional moieties to which the N-terminal truncated proHp is linked, conjugated, tethered or otherwise attached, as described elsewhere herein. In an embodiment, the additional therapeutic agent is selected from the group consisting of an immunoglobulin Fc region, or an Fc receptor binding fragment thereof, albumin or fragments thereof, hemopexin, transferrin or fragments thereof, the C-terminal peptide of human chorionic gonadotropin, an XTEN sequence, homo-amino acid repeats (HAP), proline-alanine-serine repeats (PAS), afamin, alpha-fetoprotein, Vitamin D binding protein, polypeptides capable of binding under physiological conditions to albumin or to immunoglobulin constant regions, polypeptides capable of binding to the neonatal Fc receptor (FcRn), particularly immunoglobulin constant regions and portions thereof, preferably the Fc portion of immunoglobulin, and combinations of any of the foregoing. In another embodiment, the functional moiety is selected from the group consisting of hydroxyethyl starch (HES), polyethylene glycol (PEG), polysialic acids (PSAs), elastin-like polypeptides, heparosan polymers, hyaluronic acid and albumin binding ligands, e.g., fatty acid chains, and combinations of any of the foregoing.

In an embodiment, the additional therapeutic agent is a vasodilator. Suitable vasodilators will be familiar to persons skilled in the art, illustrative examples of which include sydnone and sodium nitroprusside. Thus, in an embodiment disclosed herein, the additional therapeutic agent is selected from the group consisting of a sydnone and sodium nitroprusside.

Suitable adjunct therapy for the treatment of hemoglobinopathies, such as sickle cell disease and α- or β-thalassemia, include bone marrow transplantation and/blood transfusion. Additional therapeutic agents may be used to treat symptoms of sickle cell disease may include analgesics, antibiotics, ACE inhibitors, hydroxyurea, L-glutamine, iron chelating agents, folic acid, hemoglobin oxygen-affinity modulators (e.g., voxelotor) and antibodies (e.g., crizanluzumab).

Those skilled in the art will be aware that the invention described herein is subject to variations and modifications other than those specifically described. It is to be understood that the invention described herein includes all such variations and modifications. The invention also includes all such steps, features, methods, compositions and compounds referred to or indicated in this specification, individually or collectively, and any and all combinations of any two or more of said steps or features.

Certain embodiments of the invention will now be described with reference to the following examples, which are intended for the purpose of illustration only and are not intended to limit the scope of the generality hereinbefore described.

Sequences listing:

SEQ ID NO: 1 - Haptoglobin 2FS Human Hp isoform 1 precursor proHp; NP_005134

| | |
|---|---|
| 1 | MSALGAVIAL LLWGQLFAVD SGNDVTDIAD DGCPKPPEIA HGYVEHSVRY QCKNYYKLRT |
| 61 | EGDGVYTLND KKQWINKAVG DKLPECEADD GCPKPPEIAH GYVEHSVRYQ CKNYYKLRTE |
| 121 | GDGVYTLNNE KQWINKAVGD KLPECEAVCG KPKNPANPVQ RILGGHLDAK GSFPWQAKMV |
| 181 | SHHNLTTGAT LINEQWLLTT AKNLFLNHSE NATAKDIAPT LTLYVGKKQL VEIEKVVLHP |
| 241 | NYSQVDIGLI KLKQKVSVNE RVMPICLPSK DYAEVGRVGY VSGWGRNANF KFTDHLKYVM |
| 301 | LPVADQDQCI RHYEGSTVPE KKTPKSPVGV QPILNEHTFC AGMSKYQEDT CYGDAGSAFA |
| 361 | VHDLEEDTWY ATGILSFDKS CAVAEYGVYV KVTSIQDWVQ KTIAEN |

SEQ ID NO: 2 - Human Hp isoform 2 precursor proHp; NP_001119574

| | |
|---|---|
| 1 | MSALGAVIAL LLWGQLFAVD SGNDVTDIAD DGCPKPPEIA HGYVEHSVRY QCKNYYKLRT |
| 61 | EGDGVYTLNN EKQWINKAVG DKLPECEAVC GKPKNPANPV QRILGGHLDA KGSFPWQAKM |
| 121 | VSHHNLTTGA TLINEQWLLT TAKNLFLNHS ENATAKDIAP TLTLYVGKKQ LVEIEKVVLH |
| 181 | PNYSQVDIGL IKLKQKVSVN ERVMPICLPS KDYAEVGRVG YVSGWGRNAN FKFTDHLKYV |
| 241 | MLPVADQDQC IRHYEGSTVP EKKTPKSPVG VQPILNEHTF CAGMSKYQED TCYGDAGSAF |
| 301 | AVHDLEEDTW YATGILSFDK SCAVAEYGVY VKVTSIQDWV QKTIAEN |

SEQ ID NO: 3 - Human Hp isoform 3 precursor proHp; NP_001305067

| | |
|---|---|
| 1 | MSALGAVIAL LLWGQLFAVD SGNDVTDIAD DGCPKPPEIA HGYVEHSVRY QCKNYYKLRT |
| 61 | EGDGVYTLND KKQWINKAVG DKLPECEAVC GKPKNPANPV QRILGGHLDA KGSFPWQAKM |
| 121 | VSHHNLTTGA TLINEQWLLT TAKNLFLNHS ENATAKDIAP TLTLYVGKKQ LVEIEKVVLH |
| 181 | PNYSQVDIGL IKLKQKVSVN ERVMPICLPS KDYAEVGRVG YVSGWGRNAN FKFTDHLKYV |
| 241 | MLPVADQDQC IRHYEGSTVP EKKTPKSPVG VQPILNEHTF CAGMSKYQED TCYGDAGSAF |
| 301 | AVHDLEEDTW YATGILSFDK SCAVAEYGVY VKVTSIQDWV QKTIAEN |

SEQ ID NO: 4 - Human C1r-LP; NP_057630

| | |
|---|---|
| 1 | MPGPRVWGKY LWRSPHSKGC PGAMWWLLLW GVLQACPTRG SVLLAQELPQ QLTSPGYPEP |
| 61 | YGKGQESSTD IKAPEGFAVR LVFQDFDLEP SQDCAGDSVT ISFVGSDPSQ FCGQQGSPLG |
| 121 | RPPGQREFVS SGRSLRLTFR TQPSSENKTA HLHKGFLALY QTVAVNYSQP ISEASRGSEA |
| 181 | INAPGDNPAK VQNHCQEPYY QAAAAGALTC ATPGTWKDRQ DGEEVLQCMP VCGRPVTPIA |
| 241 | QNQTTLGSSR AKLGNFPWQA FTSIHGRGGG ALLGDRWILT AAHTIYPKDS VSLRKNQSVN |
| 301 | VFLGHTAIDE MLKLGNHPVH RVVVHPDYRQ NESHNFSGDI ALLELQHSIP LGPNVLPVCL |
| 361 | PDNETLYRSG LLGYVSGFGM EMGWLTTELK YSRLPVAPRE ACNAWLQKRQ RPEVFSDNMF |
| 421 | CVGDETQRHS VCQGDSGSVY VVWDNHAHHW VATGIVSWGI GCGEGYDFYT KVLSYVDWIK |
| 481 | GVMNGKN |

SEQ ID NO: 5 - Human C1r-LP; NP_001284569

| | |
|---|---|
| 1 | MPGPRVWGKY LWRSPHSKGC PGAMWWLLLW GVLQACPTRG SVLLAQELPQ QLTSPGYPEP |
| 61 | YGKGQESSTD IKAPEGFAVR LVFQDFDLEP SQDCAGDSVT ISFVGSDPSQ FCGQQGSPLG |
| 121 | RPPGQREFVS SGRSLRLTFR TQPSSENKTA HLHKGFLALY QTVGALTCAT PGTWKDRQDG |
| 181 | EEVLQCMPVC GRPVTPIAQN QTTLGSSRAK LGNFPWQAFT SIHGRGGGAL LGDRWILTAA |
| 241 | HTIYPKDSVS LRKNQSVNVF LGHTAIDEML KLGNHPVHRV VVHPDYRQNE SHNFSGDIAL |
| 301 | LELQHSIPLG PNVLPVCLPD NETLYRSGLL GYVSGFGMEM GWLTTELKYS RLPVAPREAC |
| 361 | NAWLQKRQRP EVFSDNMFCV GDETQRHSVC QGDSGSVYVV WDNHAHHWVA TGIVSWGIGC |
| 421 | GEGYDFYTKV LSYVDWIKGV MNGKN |

SEQ ID NO: 6 - Human C1r-LP; NP_001284571

| | |
|---|---|
| 1 | MPGPRVWGKY LWRSPHSKGC PGAMWWLLLW GVLQACPTRG SVLLAQELPQ QLTSPGYPEP |
| 61 | YGKGQESSTD IKAPEGFAVR LVFQDFDLEP SQDCAGDSVT ISFVGSDPSQ FCGQQGSPLG |
| 121 | RPPGQREFVS SGRSLRLTFR TQPSSENKTA HLHKGFLALY QTVAVNYSQP ISEASRGSEA |
| 181 | INAPGDNPAK VQNHCQEPYY QAAAAASTPS LFLCLSSFTP QGHSPVQPQG PGKTDRMGRR |
| 241 | FFSVCLSADG QSPPLPRIRR PSVLPEPSWA TSPGKPSPVS TAVGAGPCWG TDGSSLLPTP |
| 301 | STPRTVFLSG RTRV |

SEQ ID NO: 7 - Human C1r-LP; NP_001284572

| | |
|---|---|
| 1 | MPGPRVWGKY LWRSPHSKGC PGAMWWLLLW GVLQACPTRG SVLLAQELPQ QLTSPGYPEP |
| 61 | YGKGQESSTD IKAPEGFAVR LVFQDFDLEP SQDCAGDSVT ISFVGSDPSQ FCGQQGSPLG |
| 121 | RPPGQREFVS SGRSLRLTFR TQPSSENKTA HLHKGFLALY QTVGECPSWG CREGASVPSH |
| 181 | DPGIFKP |

SEQ ID NO: 8 - the 14 contiguous C-terminal amino acid residues of Hp α-chain
VCGKPKNPANPVQR
SEQ ID NO: 9 - Human Serum Albumin (HAS); NP_000468

| | |
|---|---|
| 1 | MKWVTFISLL FLFSSAYSRG VFRRDAHKSE VAHRFKDLGE ENFKALVLIA FAQYLQQCPF |
| 61 | EDHVKLVNEV TEFAKTCVAD ESAENCDKSL HTLFGDKLCT VATLRETYGE MADCCAKQEP |
| 121 | ERNECFLQHK DDNPNLPRLV RPEVDVMCTA FHDNEETFLK KYLYEIARRH PYFYAPELLF |
| 181 | FAKRYKAAFT ECCQAADKAA CLLPKLDELR DEGKASSAKQ RLKCASLQKF GERAFKAWAV |
| 241 | ARLSQRFPKA EFAEVSKLVT DLTKVHTECC HGDLLECADD RADLAKYICE NQDSISSKLK |
| 301 | ECCEKPLLEK SHCIAEVEND EMPADLPSLA ADFVESKDVC KNYAEAKDVF LGMFLYEYAR |
| 361 | RHPDYSVVLL LRLAKTYETT LEKCCAAADP HECYAKVFDE FKPLVEEPQN LIKQNCELFE |
| 421 | QLGEYKFQNA LLVRYTKKVP QVSTPTLVEV SRNLGKVGSK CCKHPEAKRM PCAEDYLSVV |
| 481 | LNQLCVLHEK TPVSDRVTKC CTESLVNRRP CFSALEVDET YVPKEFNAET FTFHADICTL |
| 541 | SEKERQIKKQ TALVELVKHK PKATKEQLKA VMDDFAAFVE KCCKADDKET CFAEEGKKLV |
| 601 | AASQAALGL |

SEQ ID NO: 10 - Human CD163; NP_981961

| | |
|---|---|
| 1 | MSKLRMVLLE DSGSADFRRH FVNLSPFTIT VVLLLSACFV TSSLGGTDKE LRLVDGENKC |
| 61 | SGRVEVKVQE EWGTVCNNGW SMEAVSVICN QLGCPTAIKA PGWANSSAGS GRIWMDHVSC |
| 121 | RGNESALWDC KHDGWGKHSN CTHQQDAGVT CSDGSNLEMR LTRGGNMCSG RIEIKFQGRW |
| 181 | GTVCDDNFNI DHASVICRQL ECGSAVSFSG SSNFGEGSGP IWFDDLICNG NESALWNCKH |
| 241 | QGWGKHNCDH AEDAGVICSK GADLSLRLVD GVTECSGRLE VRFQGEWGTI CDDGWDSYDA |
| 301 | AVACKQLGCP TAVTAIGRVN ASKGFGHIWL DSVSCQGHEP AIWQCKHHEW GKHYCNHNED |
| 361 | AGVTCSDGSD LELRLRGGGS RCAGTVEVEI QRLLGKVCDR GWGLKEADVV CRQLGCGSAL |
| 421 | KTSYQVYSKI QATNTWLFLS SCNGNETSLW DCKNWQWGGL TCDHYEEAKI TCSAHREPRL |
| 481 | VGGDIPCSGR VEVKHGDTWG SICDSDFSLE AASVLCRELQ CGTVVSILGG AHFGEGNGQI |
| 541 | WAEEFQCEGH ESHLSLCPVA PRPEGTCSHS RDVGVVCSRY TEIRLVNGKT PCEGRVELKT |
| 601 | LGAWGSLCNS HWDIEDAHVL CQQLKCGVAL STPGGARFGK GNGQIWRHMF HCTGTEQHMG |
| 661 | DCPVTALGAS LCPSEQVASV ICSGNQSQTL SSCNSSSLGP TRPTIPEESA VACIESGQLR |
| 721 | LVNGGGRCAG RVEIYHEGSW GTICDDSWDL SDAHVVCRQL GCGEAINATG SAHFGEGTGP |
| 781 | IWLDEMKCNG KESRIWQCHS HGWGQQNCRH KEDAGVICSE FMSLRLTSEA SREACAGRLE |
| 841 | VFYNGAWGTV GKSSMSETTV GVVCRQLGCA DKGKINPASL DKAMSIPMWV DNVQCPKGPD |
| 901 | TLWQCPSSPW EKRLASPSEE TWITCDNKIR LQEGPTSCSG RVEIWHGGSW GTVCDDSWDL |
| 961 | DDAQVVCQQL GCGPALKAFK EAEFGQGTGP IWLNEVKCKG NESSLWDCPA RRWGHSECGH |
| 1021 | KEDAAVNCTD ISVQKTPQKA TTGRSSRQSS FIAVGILGVV LLAIFVALFF LTKKRRQRQR |
| 1081 | LAVSSRGENL VHQIQYREMN SCLNADDLDL MNSSGGHSEP H |

SEQ ID NO: 11 - Human LRP1; NP_002323

| | |
|---|---|
| 1 | MLTPPLLLLL PLLSALVAAA IDAPKTCSPK QFACRDQITC ISKGWRCDGE RDCPDGSDEA |
| 61 | PEICPQSKAQ RCQPNEHNCL GTELCVPMSR LCNGVQDCMD GSDEGPHCRE LQGNCSRLGC |
| 121 | QHHCVPTLDG PTCYCNSSFQ LQADGKTCKD FDECSVYGTC SQLCTNTDGS FICGCVEGYL |
| 181 | LQPDNRSCKA KNEPVDRPPV LLIANSQNIL ATYLSGAQVS TITPTSTRQT TAMDFSYANE |
| 241 | TVCWVHVGDS AAQTQLKCAR MPGLKGFVDE HTINISLSLH HVEQMAIDWL TGNFYFVDDI |
| 301 | DDRIFVCNRN GDTCVTLLDL ELYNPKGIAL DPAMGKVFFT DYGQIPKVER CDMDGQNRTK |
| 361 | LVDSKIVFPH GITLDLVSRL VYWADAYLDY IEVVDYEGKG RQTIIQGILI EHLYGLTVFE |
| 421 | NYLYATNSDN ANAQQKTSVI RVNRFNSTEY QVVTRVDKGG ALHIYHQRRQ PRVRSHACEN |
| 481 | DQYGKPGGCS DICLLANSHK ARTCRCRSGF SLGSDGKSCK KPEHELFLVY GKGRPGIIRG |
| 541 | MDMGAKVPDE HMIPIENLMN PRALDFHAET GFIYFADTTS YLIGRQKIDG TERETILKDG |
| 601 | IHNVEGVAVD WMGDNLYWTD DGPKKTISVA RLEKAAQTRK TLIEGKMTHP RAIVVDPLNG |
| 661 | WMYWTDWEED PKDSRRGRLE RAWMDGSHRD IFVTSKTVLW PNGLSLDIPA GRLYWVDAFY |
| 721 | DRIETILLNG TDRKIVYEGP ELNHAFGLCH HGNYLFWTEY RSGSVYRLER GVGGAPPTVT |
| 781 | LLRSERPPIF EIRMYDAQQQ QVGTNKCRVN NGGCSSLCLA TPGSRQCACA EDQVLDADGV |
| 841 | TCLANPSYVP PPQCQPGEFA CANSRCIQER WKCDGDNDCL DNSDEAPALC HQHTCPSDRF |
| 901 | KCENNRCIPN RWLCDGDNDC GNSEDESNAT CSARTCPPNQ FSCASGRCIP ISWTCDLDDD |
| 961 | CGDRSDESAS CAYPTCFPLT QFTCNNGRCI NINWRCDNDN DCGDNSDEAG CSHSCSSTQF |
| 1021 | KCNSGRCIPE HWTCDGDNDC GDYSDETHAN CTNQATRPPG GCHTDEFQCR LDGLCIPLRW |
| 1081 | RCDGDTDCMD SSDEKSCEGV THVCDPSVKF GCKDSARCIS KAWVCDGDND CEDNSDEENC |
| 1141 | ESLACRPPSH PCANNTSVCL PPDKLCDGND DCGDGSDEGE LCDQCSLNNG GCSHNCSVAP |
| 1201 | GEGIVCSCPL GMELGPDNHT CQIQSYCAKH LKCSQKCDQN KFSVKCSCYE GWVLEPDGES |
| 1261 | CRSLDPFKPF IIFSNRHEIR RIDLHKGDYS VLVPGLRNTI ALDFHLSQSA LYWTDVVEDK |
| 1321 | IYRGKLLDNG ALTSFEVVIQ YGLATPEGLA VDWIAGNIYW VESNLDQIEV AKLDGTLRTT |
| 1381 | LLAGDIEHPR AIALDPRDGI LFWTDWDASL PRIEAASMSG AGRRTVHRET GSGGWPNGLT |
| 1441 | VDYLEKRILW IDARSDAIYS ARYDGSGHME VLRGHEFLSH PFAVTLYGGE VYWTDWRTNT |
| 1501 | LAKANKWTGH NVTVVQRTNT QPFDLQVYHP SRQPMAPNPC EANGGQGPCS HLCLINYNRT |
| 1561 | VSCACPHLMK LHKDNTTCYE FKKFLLYARQ MEIRGVDLDA PYYNYIISFT VPDIDNVTVL |
| 1621 | DYDAREQRVY WSDVRTQAIK RAFINGTGVE TVVSADLPNA HGLAVDWVSR NLFWTSYDTN |
| 1681 | KKQINVARLD GSFKNAVVQG LEQPHGLVVH PLRGKLYWTD GDNISMANMD GSNRTLLFSG |
| 1741 | QKGPVGLAID FPESKLYWIS SGNHTINRCN LDGSGLEVID AMRSQLGKAT ALAIMGDKLW |
| 1801 | WADQVSEKMG TCSKADGSGS VVLRNSTTLV MHMKVYDESI QLDHKGTNPC SVNNGDCSQL |
| 1861 | CLPTSETTRS CMCTAGYSLR SGQQACEGVG SFLLYSVHEG IRGIPLDPND KSDALVPVSG |
| 1921 | TSLAVGIDFH AENDTIYWVD MGLSTISRAK RDQTWREDVV TNGIGRVEGI AVDWIAGNIY |
| 1981 | WTDQGFDVIE VARLNGSFRY VVISQGLDKP RAITVHPEKG YLFWTEWGQY PRIERSRLDG |
| 2041 | TERVVLVNVS ISWPNGISVD YQDGKLYWCD ARTDKIERID LETGENREVV LSSNNMDMFS |
| 2101 | VSVFEDFIYW SDRTHANGSI KRGSKDNATD SVPLRTGIGV QLKDIKVFNR DRQKGTNVCA |
| 2161 | VANGGCQQLC LYRGRGQRAC ACAHGMLAED GASCREYAGY LLYSERTILK SIHLSDERNL |
| 2221 | NAPVQPFEDP EHMKNVIALA FDYRAGTSPG TPNRIFFSDI HFGNIQQIND DGSRRITIVE |
| 2281 | NVGSVEGLAY HRGWDTLYWT SYTTSTITRH TVDQTRPGAF ERETVITMSG DDHPRAFVLD |
| 2341 | ECQNLMFWTN WNEQHPSIMR AALSGANVLT LIEKDIRTPN GLAIDHRAEK LYFSDATLDK |
| 2401 | IERCEYDGSH RYVILKSEPV HPFGLAVYGE HIFWTDWVRR AVQRANKHVG SNMKLLRVDI |
| 2461 | PQQPMGIIAV ANDTNSCELS PCRINNGGCQ DLCLLTHQGH VNCSCRGGRI LQDDLTCRAV |
| 2521 | NSSCRAQDEF ECANGECINF SLTCDGVPHC KDKSDEKPSY CNSRRCKKTF RQCSNGRCVS |
| 2581 | NMLWCNGADD CGDGSDEIPC NKTACGVGEF RCRDGTCIGN SSRCNQFVDC EDASDEMNCS |
| 2641 | ATDCSSYFRL GVKGVLFQPC ERTSLCYAPS WVCDGANDCG DYSDERDCPG VKRPRCPLNY |
| 2701 | FACPSGRCIP MSWTCDKEDD CEHGEDETHC NKFCSEAQFE CQNHRCISKQ WLCDGSDDCG |
| 2761 | DGSDEAAHCE GKTCGPSSFS CPGTHVCVPE RWLCDGDKDC ADGADESIAA GCLYNSTCDD |
| 2821 | REFMCQNRQC IPKHFVCDHD RDCADGSDES PECEYPTCGP SEFRCANGRC LSSRQWECDG |
| 2881 | ENDCHDQSDE APKNPHCTSQ EHKCNASSQF LCSSGRCVAE ALLCNGQDDC GDSSDERGCH |
| 2941 | INECLSRKLS GCSQDCEDLK IGFKCRCRPG FRLKDDGRTC ADVDECSTTF PCSQRCINTH |
| 3001 | GSYKCLCVEG YAPRGGDPHS CKAVTDEEPF LIFANRYYLR KLNLDGSNYT LLKQGLNNAV |
| 3061 | ALDFDYREQM IYWTDVTTQG SMIRRMHLNG SNVQVLHRTG LSNPDGLAVD WVGGNLYWCD |
| 3121 | KGRDTIEVSK LNGAYRTVLV SSGLREPRAL VVDVQNGYLY WTDWGDHSLI GRIGMDGSSR |
| 3181 | SVIVDTKITW PNGLTLDYVT ERIYWADARE DYIEFASLDG SNRHVVLSQD IPHIFALTLF |
| 3241 | EDYVYWTDWE TKSINRAHKT TGTNKTLLIS TLHRPMDLHV FHALRQPDVP NHPCKVNNGG |
| 3301 | CSNLCLLSPG GGHKCACPTN FYLGSDGRTC VSNCTASQFV CKNDKCIPFW WKCDTEDDCG |
| 3361 | DHSDEPPDCP EFKCRPGQFQ CSTGICTNPA FICDGDNDCQ DNSDEANCDI HVCLPSQFKC |
| 3421 | TNTNRCIPGI FRCNGQDNCG DGEDERDCPE VTCAPNQFQC SITKRCIPRV WVCDRDNDCV |
| 3481 | DGSDEPANCT QMTCGVDEFR CKDSGRCIPA RWKCDGEDDC GDGSDEPKEE CDERTCEPYQ |
| 3541 | FRCKNNRCVP GRWQCDYDND CGDNSDEESC TPRPCSESEF SCANGRCIAG RWKCDGDHDC |
| 3601 | ADGSDEKDCT PRCDMDQFQC KSGHCIPLRW RCDADADCMD GSDEEACGTG VRTCPLDEFQ |
| 3661 | CNNTLCKPLA WKCDGEDDCG DNSDENPEEC ARFVCPPNRP FRCKNDRVCL WIGRQCDGTD |
| 3721 | NCGDGTDEED CEPPTAHTTH CKDKKEFLCR NQRCLSSSLR CNMFDDCGDG SDEEDCSIDP |
| 3781 | KLTSCATNAS ICGDEARCVR TEKAAYCACR SGFHTVPGQP GCQDINECLR FGTCSQLCNN |
| 3841 | TKGGHLCSCA RNFMKTHNTC KAEGSEYQVL YIADDNEIRS LFPGHPHSAY EQAFQGDESV |
| 3901 | RIDAMDVHVK AGRVYWTNWH TGTISYRSLP PAAPPTTSNR HRRQIDRGVT HLNISGLKMP |
| 3961 | RGIAIDWVAG NVYWTDSGRD VIEVAQMKGE NRKTLISGMI DEPHAIVVDP LRGTMYWSDW |
| 4021 | GNHPKIETAA MDGTLRETLV QDNIQWPTGL AVDYHNERLY WADAKLSVIG SIRLNGTDPI |
| 4081 | VAADSKRGLS HPFSIDVFED YIYGVTYINN RVFKIHKFGH SPLVNLTGGL SHASDVVLYH |
| 4141 | QHKQPEVTNP CDRKKCEWLC LLSPSGPVCT CPNGKRLDNG TCVPVPSPTP PPDAPRPGTC |
| 4201 | NLQCFNGGSC FLNARRQPKC RCQPRYTGDK CELDQCWEHC RNGGTCAASP SGMPTCRCPT |
| 4261 | GFTGPKCTQQ VCAGYCANNS TCTVNQGNQP QCRCLPGFLG DRCQYRQCSG YCENFGTCQM |
| 4321 | AADGSRQCRC TAYFEGSRCE VNKCSRCLEG ACVVNKQSGD VTCNCTDGRV APSCLTCVGH |
| 4381 | CSNGGSCTMN SKMMPECQCP PHMTGPRCEE HVFSQQQPGH IASILIPLLL LLLLVLVAGV |
| 4441 | VFWYKRRVQG AKGFQHQRMT NGAMNVEIGN PTYKMYEGGE PDDVGGLLDA DFALDPDKPT |
| 4501 | NFTNPVYATL YMGGHGSRHS LASTDEKREL LGRGPEDEIG DPLA |

SEQ ID NO: 12 - Human hemopexin (Hpx); NP_000604

| | |
|---|---|
| 1 | MARVLGAPVA LGLWSLCWSL AIATPLPPTS AHGNVAEGET KPDPDVTERC SDGWSFDATT |
| 61 | LDDNGTMLFF KGEFVWKSHK WDRELISERW KNFPSPVDAA FRQGHNSVFL IKGDKVWVYP |
| 121 | PEKKEKGYPK LLQDEFPGIP SPLDAAVECH RGECQAEGVL FFQGDREWFW DLATGTMKER |
| 181 | SWPAVGNCSS ALRWLGRYYC FQGNQFLRFD PVRGEVPPRY PRDVRDYFMP CPGRGHGHRN |
| 241 | GTGHGNSTHH GPEYMRCSPH LVLSALTSDN HGATYAFSGT HYWRLDTSRD GWHSWPIAHQ |
| 301 | WPQGPSAVDA AFSWEEKLYL VQGTQVYVFL TKGGYTLVSG YPKRLEKEVG TPHGIILDSV |
| 361 | DAAFICPGSS RLHIMAGRRL WWLDLKSGAQ ATWTELPWPH EKVDGALCME KSLGPNSCSA |
| 421 | NGPGLYLIHG PNLYCYSDVE KLNAAKALPQ PQNVTSLLGC TH |

SEQ ID NO: 13 - Hu-LRPAP1; NP_002328

| | |
|---|---|
| 1 | MAPRRVRSFL RGLPALLLLL LFLGPWPAAS HGGKYSREKN QPKPSPKRES GEEFRMEKLN |
| 61 | QLWEKAQRLH LPPVRLAELH ADLKIQERDE LAWKKLKLDG LDEDGEKEAR LIRNLNVILA |
| 121 | KYGLDGKKDA RQVTSNSLSG TQEDGLDDPR LEKLWHKAKT SGKFSGEELD KLWREFLHHK |
| 181 | EKVHEYNVLL ETLSRTEEIH ENVISPSDLS DIKGSVLHSR HTELKEKLRS INQGLDRLRR |
| 241 | VSHQGYSTEA EFEEPRVIDL WDLAQSANLT DKELEAFREE LKHFEAKIEK HNHYQKQLEI |
| 301 | AHEKLRHAES VGDGERVSRS REKHALLEGR TKELGYTVKK HLQDLSGRIS RARHNEL |

SEQ ID NO: 14 - amino acid sequences that are common to the -chain of Hp1 and Hp2
VDSGNDVTDIADDGCPKPPEIAHGYVEHSVRYQCKNYYKLRTEGDGVYTLN
SEQ ID NO: 15 - amino acid sequence of human lgG4 Fc region
ESKYGPPCPPCPAPEFLGGPSVFLFPPKPKDTLMISRTPEVTCVVVDVSQEDPEVQFNWYV
DGVEVHNAKTKPREEQFNSTYRVVSVLTVLHQDWLNGKEYKCKVSNKGLPSSIEKTISKAK
GQPREPQVYTLPPSQEEMTKNQVSLTCLVKGFYPSDIAVEWESNGQPENNYKTTPPVLDS
DGSFFLYSRLTVDKSRWQEGNVFSCSVMHEALHNHYTQKSLSLSLGK
SEQ ID NO: 16 - amino acid sequence of mouse IgG2a Fc region
APNLLGGPSVFIFPPKIKDVLMISLSPIVTCVVVDVSEDDPDVQISWFVNNVEVHTAQTQTHR
EDYNSTLRVVSALPIQHQDWMSGKEFKCKVNNKDLPAPIERTISKPKGSVRAPQVYVLPPPE
EEMTKKQVTLTCMVTDFMPEDIYVEWTNNGKTELNYKNTEPVLDSDGSYFMYSKLRVEKK
NWVERNSYSCSVVHEGLHNHHTTKSFSRTPGK
SEQ ID NO: 17 - amino acid sequences that are common to the α-chain of Hp1 and Hp2
NEKQWINKAVGDKLPECEAVCGKPKNPANPVQR

### EXAMPLES

### A. Abbreviations

Hp Haptoglobin
Hpx Hemopexin
Hb Hemoglobin
HSA Human Serum Albumin
C1r-LP C1r subcomponent like protein
BLI Bilayer Inferometry
SPR Surface Plasmon Resonance
8His 8 Histidine tag (SEQ ID NO: 18)
Fc Fragment crystallizable

### B. General Procedures

### B.1 Cell Culture

Expi293F™ cells and the mammalian expression vector pcDNA3.1 were obtained from Invitrogen™, Thermo Fisher Scientific (R790-07, V790-20). Cells were cultured in GIBCO® Expi 293 Expression Medium (Invitrogen™, Thermo Fisher Scientific). All tissue culture media were supplemented with Antibiotic-Antimycotic (GIBCO®, Thermo Fisher Scientific 15240-096) and cells were maintained at 37° C. in incubators with an atmosphere of 8% CO₂.

### B.2 Antibodies

His Tag Antibody [FITC], GenScript, Cat #A01620
Goat Anti-Human IgG [FITC]Southern Biotech
Polyclonal Antibody to Haptoglobin, Acris Antibodies Cat #AP08546PU-N
   B.3 Generation of cDNA Plasmids

The amino acid sequence of various proteins used herein are recorded in the Genbank® database and are assigned the accession numbers (see Table 1, below).

TABLE 1
Amino acid sequences and / or Genbank® accession numbers

| Protein | Accession No. | SEQ ID NO: |
|---|---|---|
| Haptoglobin 2FS | NP_005134 | 1 |
| Human Hp isoform 1 | | |
| precursor proHp | | |
| Human Hp isoform 2 | NP_001119574 | 2 |
| precursor proHp | | |
| Human Hp isoform 3 | NP_001305067 | 3 |
| precursor proHp | | |
| Human C1r-LP | NP_057630 | 4 |
| 14 contiguous C- | VCGKPKNPANPVQR | 8 |
| terminal amino acid | | |
| residues of the Hp α- | | |
| chain | | |
| HSA | NP_000468 | 9 |
| Human CD163 | NP_981961 | 10 |
| Human LRP1 | NP_002323.2 | 11 |
| Human Hpx | NP_000604 | 12 |
| Hu-LRPAP1 | NP_002328.1 | 13 |
| amino acid sequences | VDSGNDVTDIADDGCPKPPEIAHGYVEHSVRYQ | 14 |
| common to the α-chain | CKNYYKLRTEGDGVYTLN | |
| of Hp1 and Hp2 | | |
| Human IgG4 Fc | ESKYGPPCPPCPAPEFLGGPSVFLFPPKPKDTL | 15 |
| | MISRTPEVTCVVVDVSQEDPEVQFNWYVDGVE | |
| | VHNAKTKPREEQFNSTYRVVSVLTVLHQDWLN | |
| | GKEYKCKVSNKGLPSSIEKTISKAKGQPREPQV | |
| | YTLPPSQEEMTKNQVSLTCLVKGFYPSDIAVEW | |
| | ESNGQPENNYKTTPPVLDSDGSFFLYSRLTVDK | |
| | SRWQEGNVFSCSVMHEALHNHYTQKSLSLSLG | |
| | K | |
| Mouse IgG2a Fc | APNLLGGPSVFIFPPKIKDVLMISLSPIVTCVVVD | 16 |
| | VSEDDPDVQISWFVNNVEVHTAQTQTHREDYN | |
| | STLRVVSALPIQHQDWMSGKEFKCKVNNKDLP | |
| | APIERTISKPKGSVRAPQVYVLPPPEEEMTKKQV | |
| | TLTCMVTDFMPEDIYVEWTNNGKTELNYKNTEP | |
| | VLDSDGSYFMYSKLRVEKKNWVERNSYSCSVV | |
| | HEGLHNHHTTKSFSRTPGK | |
| amino acid sequences | NEKQWINKAVGDKLPECEAVCGKPKNPANPVQ | 17 |
| common to the α-chain | R | |
| of Hp1 and Hp2 | | |

cDNAs were codon-optimized for human expression and synthesized by Geneart® (Invitrogen™, Thermo Fisher Scientific) each with a Kozak consensus sequence (Kozak 1987) (GCCACC) immediately upstream of the initiating methionine (+1). Variant molecules were generated using standard PCR-based mutagenesis techniques. Once each cDNA was completed, it was digested with NheI and XhoI and ligated into pcDNA3.1 (Invitrogen™ Thermo Fisher Scientific). Large-scale preparations of plasmid DNA were carried out using QIAGEN Plasmid Giga Kits (12191) according to the manufacturer's instructions. The nt sequences of all plasmid constructs were verified by sequencing both strands using BigDye™ Terminator Version 3.1 Ready Reaction Cycle Sequencing (Invitrogen™, Thermo Fisher Scientific) and an Applied Biosystems 3130xl Genetic Analyzer.

### B.4 Transient Transfections for Generation of Recombinant Proteins

### Expi293F

Transient transfections of expression plasmids using Expi293F cells were performed using Expifectamine™ transfection reagent (Invitrogen™, Life Technologies) according to the manufacturer's instructions. Cells were transfected at a final concentration of 1×10⁶ viable cells/ml and incubated in a shaking incubator (Infors) for 6 days at 37° C. in 8% CO₂. Pluronic F68 (GIBCO, Life Technologies), to a final concentration of 0.1% v/v, was added 4 h post-transfection. At 24 h post-transfection, cell cultures were supplemented with LucraTone Lupin (Millipore) to a final concentration of 0.5% v/v. The cell culture supernatants were harvested by centrifugation at 2500 rpm and were then passed through a 0.45 μm filter (Nalgene) prior to purification.

### ExpiCHO

Transient transfections of expression plasmids encoding huLRP1 soluble minireceptor binding domain III (90%) together with human LDL Receptor Related Protein Associated Protein 1 (huLRPAP1,RAP, 10%) using ExpiCHO-S™ cells were performed using Expifectamine™ transfection reagent (Invitrogen, Life Technologies) according to the manufacturer's instructions. Cells were transfected at a final concentration of 6×10⁶ viable cells/ml and incubated in a shaking incubator (Infors) for 20 hours at 37° C. in 8% CO2. After 20 hours, Enhancer™ and a Feed™ was added to the cultures. The culture is then incubated at 32° C., 5% CO2, 70% humidity for a further 5 days. At day 5 post-transfection a second Feed™ was added to the cultures and they were returned to the incubator at 32° C., 5% CO2, 70% humidity. The cell culture supernatants were harvested by centrifugation at 2500 rpm and were then passed through a 0.45 μm filter (Nalgene) prior to purification. Expression of recombinant huLRP1 soluble minireceptor in the culture supernatants was confirmed by SDS-PAGE (NuPAGE system, Thermo Fisher Scientific, MA, USA) and also by Western blot analysis using an anti-His antibody (His Tag Antibody [FITC], GenScript, Cat #A01620).

### B.5 Purification of His-Tagged Proteins

### Hp(148-406) Variants

His-tagged recombinant Hp(148-406) variants were purified on an ÄKTAxpress system (Cytiva) using an automated method for tandem chromatography. Specifically, 30 ml Expi293F supernatant was loaded onto a 1 ml HisTrap Excel column (Cytiva) equilibrated in 10 mM imidazole; 20 mM NaH₂PO₄; 500 mM NaCl (pH 7.4). The bound His-tagged proteins were subsequently washed with 25 mM imidazole; 20 mM NaH₂PO₄; 500 mM NaCl (pH 7.4) to reduce non-specifically interacting proteins prior to elution into a holding loop using 500 mM imidazole; 20 mM NaH₂PO₄; 500 mM NaCl (pH 7.4). The eluate captured from the HisTrap excel column was then injected onto a HiPrep 26/10 desalting column (Cytiva) for buffer exchange into MT-PBS.

Protein-containing fractions containing all size species were pooled and concentrated using Amicon Ultra-15 centrifugal ultrafiltration devices (Merck-Millipore, MS, USA) prior to passage through a 0.22 um filter. Protein concentration was then measured by OD280 using a Trinean DropSense96 system (Trinean) and the purity was verified by SDS-PAGE separation on a NuPAGE 4-12% Bis-Tris gel (Thermo Fisher Scientific). The level of higher order species in solution was assessed using an analytical Superdex 200 Increase (15/50) size exclusion column connected to an Agilent 1260 Infinity HPLC with MT-PBS as the mobile phase. 1 ul Aqueous SEC1 (ALO-3042) molecular weight standards from Phenomenex were run as part of the analysis and overlaid for comparison.

### Hp(162-406) Variants

His-tagged recombinant Hp(162-406) variants were purified on an ÄKTAxpress system (Cytiva) using an automated method for tandem chromatography. Specifically, 1-2 L of Expi293F supernatant was loaded onto a 5 ml HisTrap Excel column (Cytiva) equilibrated in 10 mM imidazole; 20 mM NaH₂PO₄; 500 mM NaCl (pH 7.4). The bound His-tagged proteins were subsequently washed with 25 mM imidazole; 20 mM NaH₂PO₄; 500 mM NaCl (pH 7.4) to reduce non-specifically interacting proteins prior to elution into a holding loop using 500 mM imidazole; 20 mM NaH₂PO₄; 500 mM NaCl (pH 7.4). The eluate captured from the HisTrap excel column was then injected onto a Superdex 200 26/60 HiPrep size exclusion column (Cytiva) for the preparative separation of aggregate and size species in a MT-PBS mobile phase.

Fractions containing proteins of the expected size were pooled and concentrated using Amicon Ultra-15 centrifugal ultrafiltration devices (Merck-Millipore, MS, USA) prior to passage through a 0.22 um filter. Protein concentration was then measured by OD280 using a Trinean DropSense96 system (Trinean) and the purity was verified by SDS-PAGE separation on a NuPAGE 4-12% Bis-Tris gel (Thermo Fisher Scientific). The level higher order species in solution was assessed using an analytical Superdex 200 Increase (15/50) size exclusion column connected to an Agilent 1260 Infinity HPLC with MT-PBS as the mobile phase. 1 ul Aqueous SEC1 (ALO-3042) molecular weight standards from Phenomenex were run as part of the analysis and overlaid for comparison.

### B.6 Purification of Albumin Fusion Proteins

### Murine Albumin Fusion Proteins

### Hp(148-406) Variants

Murine albumin (MSA)-fused Hp(148-406) variants were purified on an ÄKTAxpress system (Cytiva) using an automated method for tandem chromatography. Specifically, 30 ml Expi293F supernatant was loaded onto a 5 ml Mimetic Blue multi species albumin affinity column (Astrea Bioseparations) equilibrated in 10 mM TRIS; 150 mM NaCl (pH 7.5). The bound MSA fusion proteins were subsequently washed with 10 mM TRIS; 150 mM NaCl (pH 7.5) to reduce non-specifically interacting proteins prior to a elution into a holding loop using 30 mM octanoate; 10 mM TRIS; 150 NaCl (pH 7.4). The bound MSA fusion proteins were subsequently washed with 10 mM TRIS; 150 mM NaCl (pH 7.5) to reduce non-specifically interacting proteins prior to a elution into a holding loop using 30 mM octanoate; 10 mM TRIS; 150 NaCl (pH 7.4). The eluate captured from the Mimetic Blue column was then injected onto a HiPrep 26/10 desalting column (Cytiva) for buffer exchange into MT-PBS.

Protein-containing fractions containing all size species were pooled and concentrated using Amicon Ultra-15 centrifugal ultrafiltration devices (Merck-Millipore, MS, USA) prior to passage through a 0.22 um filter. Protein concentration was then measured by OD280 using a Trinean DropSense96 system (Trinean) and the purity was verified by SDS-PAGE separation on a NuPAGE 4-12% Bis-Tris gel (Thermo Fisher Scientific). The level higher order species in solution was assessed using a Superdex 200 Increase (15/50) size exclusion column connected to an Agilent 1260 Infinity HPLC with MT-PBS as the mobile phase. 1 ul Aqueous SEC1 (ALO-3042) molecular weight standards from Phenomenex were run as part of the analysis and overlaid for comparison.

### Hp(162-406) Variants

MSA-fused Hp(162-406) variants were purified on an ÄKTAxpress system (Cytiva) using an automated method for tandem chromatography. Specifically, 1-2 L of Expi293F supernatant was loaded onto a 5 ml Mimetic Blue multi species albumin affinity column (Astrea Bioseparations) equilibrated in 10 mM TRIS; 150 mM NaCl (pH 7.5). The bound MSA fusion proteins were subsequently washed with 10 mM TRIS; 150 mM NaCl (pH 7.5) to reduce non-specifically interacting proteins prior to a elution into a holding loop using 30 mM octanoate; 10 mM TRIS; 150 NaCl (pH 7.4). The eluate captured from the Mimetic Blue column was then injected onto a Superdex 200 26/60 HiPrep size exclusion column (Cytiva) for the preparative separation of aggregate and size species in a MT-PBS mobile phase.

Fractions containing proteins of the expected size were pooled and concentrated using Amicon Ultra-15 centrifugal ultrafiltration devices (Merck-Millipore) prior to passage through a 0.22 um filter. Protein concentration was then measured by OD280 using a Trinean DropSense96 system (Trinean) and the purity was verified by SDS-PAGE separation on a NuPAGE 4-12% Bis-Tris gel (Thermo Fisher Scientific). The level higher order species in solution was assessed using an analytical Superdex 200 Increase (15/50) size exclusion column connected to an Agilent 1260 Infinity HPLC with MT-PBS as the mobile phase. 1 ul Aqueous SEC1 (ALO-3042) molecular weight standards from Phenomenex were run as part of the analysis and overlaid for comparison.

### Human Albumin Fusion Proteins

### Hp(148-406) Variants

Human albumin (HSA)-fused Hp(148-406) variants were purified on Janus G3 liquid handler (Perkin Elmer) using an automated method for tandem chromatography. Specifically, 3.5 ml Expi293F supernatant was loaded onto a 200 pl CaptureSelect HSA affinity column (Thermo) equilibrated in 10 mM TRIS; 150 mM NaCl (pH 7.5). The bound HSA fusion proteins were subsequently washed with 10 mM TRIS; 150 mM NaCl (pH 7.5) to reduce non-specifically interacting proteins prior to a elution in 2 M MgCl₂; 20 mM TRIS (pH 7.4). The eluate collected from the CaptureSelect HSA affinity column was then dispensed onto a CentriPure 96 desalting array (emp Biotech GmbH) for buffer exchange into MT-PBS. The desalted samples were not further concentrated.

Protein concentration was then measured by OD280 using a Trinean DropSense96 system (Trinean) and the purity was verified by SDS-PAGE separation on a NuPAGE 4-12% Bis-Tris gel (Thermo Fisher Scientific). The level higher order species in solution was assessed using a Superdex 200 Increase (15/50) size exclusion column connected to an Agilent 1260 Infinity HPLC with MT-PBS as the mobile phase. 1 ul Aqueous SEC1 (ALO-3042) molecular weight standards from Phenomenex were run as part of the analysis and overlaid for comparison.

### Hp(162-406) Variants

HSA-fused Hp(162-406) variants were purified on an ÄKTAxpress system (Cytiva) using an automated method for tandem chromatography. Specifically, 1-2 L of Expi293F supernatant was loaded onto a 5 ml CaptureSelect HSA affinity column (Thermo) equilibrated in 10 mM TRIS; 150 mM NaCl (pH 7.5). The bound HSA fusion proteins were subsequently washed with 10 mM TRIS; 150 mM NaCl (pH 7.5) to reduce non-specifically interacting proteins prior to a elution into a holding loop using 2 M; 20 mM TRIS (pH 7.4). The eluate captured from the CaptureSelect HSA affinity column was then injected onto a Superdex 200 26/60 HiPrep size exclusion column (Cytiva) for the preparative separation of aggregate and size species in a MT-PBS mobile phase.

Fractions containing proteins of the expected size were pooled and concentrated using Amicon Ultra-15 centrifugal ultrafiltration devices (Merck-Millipore) prior to passage through a 0.22 um filter. Protein concentration was then measured by OD280 using a Trinean DropSense96 system (Trinean) and the purity was verified by SDS-PAGE separation on a NuPAGE 4-12% Bis-Tris gel (Thermo Fisher Scientific). The level higher order species in solution was assessed using an analytical Superdex 200 Increase (15/50) size exclusion column connected to an Agilent 1260 Infinity HPLC with MT-PBS as the mobile phase. 1 ul Aqueous SEC1 (ALO-3042) molecular weight standards from Phenomenex were run as part of the analysis and overlaid for comparison.

### B.7 Purification of Fc Fusion Proteins

### Hp(148-406) Variants

Hp Fc-fused variants were purified on an ÄKTAxpress system (Cytiva) using an automated method for tandem chromatography. Specifically, 30 ml Expi293F supernatant was loaded onto a 1 ml MabSelect SuRe pcc column (Cytiva) equilibrated in MT-PBS. The bound Fc fusion proteins were subsequently washed with 500 mM L-Arg; 10 mM TRIS; 150 mM NaCl (pH 7.5) to reduce aggregate and endotoxin prior to a elution into a holding loop using 0.1 M sodium acetate (pH 3.0). The eluate captured from the MabSelect SuRe pcc column was then injected onto a Superdex 200 16/60 size exclusion column (Cytiva), equilibrated in MT-PBS, to separate the Hp species by size, equilibrated in MT-PBS, to separate the Hp species by size. The eluate captured from the Mimetic Blue column was then injected onto a HiPrep 26/10 desalting column (Cytiva) for buffer exchange into MT-PBS.

Protein-containing fractions containing all size species were pooled and concentrated using Amicon Ultra-15 centrifugal ultrafiltration devices (Merck-Millipore, MS, USA) prior to passage through a 0.22 um filter. Protein concentration was then measured by OD280 using a Trinean DropSense96 system (Trinean) and the purity was verified by SDS-PAGE separation on a NuPAGE 4-12% Bis-Tris gel (Thermo Fisher Scientific). The level higher order species in solution was assessed using a Superdex 200 Increase (15/50) size exclusion column connected to an Agilent 1260 Infinity HPLC with MT-PBS as the mobile phase. 1 ul Aqueous SEC1 (ALO-3042) molecular weight standards from Phenomenex were run as part of the analysis and overlaid for comparison

### Hp(162-406) Variants

Hp Fc-fused variants were purified on an ÄKTAxpress system (Cytiva) using an automated method for tandem chromatography. Specifically, 30 ml Expi293F supernatant was loaded onto a 5 ml MabSelect SuRe pcc column (Cytiva) equilibrated in MT-PBS. The bound Fc fusion proteins were subsequently washed with 500 mM L-Arg; 10 mM TRIS; 150 mM NaCl (pH 7.5) to reduce aggregate and endotoxin prior to a elution into a holding loop using 0.1 M sodium acetate (pH 3.0). The eluate captured from the MabSelect SuRe pcc column was then injected onto a Superdex 200 16/60 size exclusion column (Cytiva), equilibrated in MT-PBS, to separate the Hp species by size. The eluate captured from the Mimetic Blue column was then injected onto a Superdex 200 26/60 HiPrep size exclusion column (Cytiva) for the preparative separation of aggregate and size species in a MT-PBS mobile phase.

Fractions containing proteins of the expected size were pooled and concentrated using Amicon Ultra-15 centrifugal ultrafiltration devices (Merck-Millipore) prior to passage through a 0.22 um filter. Protein concentration was then measured by OD280 using a Trinean DropSense96 system (Trinean) and the purity was verified by SDS-PAGE separation on a NuPAGE 4-12% Bis-Tris gel (Thermo Fisher Scientific). The level higher order species in solution was assessed using an analytical Superdex 200 Increase (15/50) size exclusion column connected to an Agilent 1260 Infinity HPLC with MT-PBS as the mobile phase. 1 ul Aqueous SEC1 (ALO-3042) molecular weight standards from Phenomenex were run as part of the analysis and overlaid for comparison.

### B.8 Qualitative Measurement of Hemoglobin Binding to Novel Proteins

Hb binding proteins were incubated with human haemoglobin (HbA) for 1 h at 37° C. at different concentrations. Hp-bound and unbound fractions of Hb (cell-free Hb) were determined by SEC-high-performance liquid chromatography (SEC-HPLC) using an Ultimate 3000SD HPLC attached to a LPG-3400SD quaternary pump and a photodiode array detector (DAD) (ThermoFisher). Plasma samples and Hb standards were separated on a Diol-300 (3 μm, 300×8.0 mm) column (YMC CO Ltd.) with PBS, pH 7.4 (Bichsel) as the mobile phase at a flow rate of 1 mL/min. For all samples two wavelengths were recorded (λ=280 nm and λ=414 nm). Bound and unbound Hb in plasma was determined by calculating the peak area of both peaks (6 min retention time for Hb:Hp, 8 min retention time for cell free Hb).

### B.9 Biotinylation of Haptoglobin

Biotinylation was performed with EZ-Link™ NHS-PEG Solid Phase Biotinylation Kit (Cat No:21450, Thermo Scientific) according to the manufacturing protocol. Briefly, protein was diluted in PBS to a concentration between 0.5-0.2 mg/ml. Distilled water was added to NHS-PEG4—Biotin to generate a 1 mM solution. To each protein of interest add the appropriated volume of 1 mM Biotin reagent calculated as follows:

$$μL biotin ( 1 mM ) = \frac{protein concentration ( mg / mL )}{M W Protein ( kDa )} \times MCR \times volume protein ( μg / mL )$$

The reaction was mixed immediately and incubated at RT for 30 min. Stop the reaction by removing the excess of biotin reagent using a desalting column equilibrated by centrifugating it at 1000×g for 2 min 3 times. Place a new collection tube and slowly apply biotinylated sample to the center of the compact resin bed and centrifuge at 1000×g for 2 min to collect the sample. The protein concentration was calculated.

### B.10 Quantitative Measurement of Hemoglobin Binding to Novel Proteins

Streptavidin pre-coated biosensors (Cat No: 18-5019, FortéBio) were used. The different Hp variants were biotinylated as described above and were immobilized in assay buffer (PBS, 0.01% BSA, 0.002% Tween20) at a concentration as indicated for each experiment. Hp variants were diluted in assay buffer (PBS, 0.01% BSA, 0.002% Tween20). The association and dissociation kinetics of Hp variants, were performed at Hb concentrations as indicated for each experiment. The settings for each binding step were chosen as shown in the Table 2Table 2. Experimental OctetRED96 settings for kinetic assessment used for HuHaptoglobin2FS(148-406)-8His* (SEQ ID NO: 18) as an example for HuHaptoglobin2FS(148-406)-8His (SEQ ID NO: 18). A reference control was included in every experiment (sensor loaded with ligand without analyte). Data was acquired on an OctetRED96 (FortéBio) at 30° C. with the following settings:

TABLE 2
Experimental OctetRED96 settings for kinetic assessment
used for HuHaptoglobin2FS(148-406)-8His* (SEQ ID NO: 18)

| Step | Time (s) | Shake speed (rpm) |
|---|---|---|
| Equilibration (buffer) | 60 | 1000 |
| Loading (Hp variant as indicated) | 600 | 1000 |
| 2x Baseline (buffer) | 60 | 1000 |
| Association (Hp variants) | 600 | 1000 |
| Dissociation (buffer) | 2400 | 1000 |

Data was analyzed by the Data Analysis Software (FortéBio, Version 9.0). Data was processed by performing baseline alignment to the y-axis, inter-step correction, reference sensors subtraction and curve smoothening by Savitzky-Golay Filtering. The processed kinetic dataset was globally fitted using a 1:1 binding model. The fitting accuracy was described by Chi² and R², parameters representing how well the measured results resemble those calculated from the model used to analyze the data.

### B.11 Measurement of Heme Binding to Novel Proteins

The heme binding method is described in (Lipiski, 2013) and was slightly adapted. Briefly, heme-albumin (12.5 μM in PBS) was incubated with heme binding proteins (eg. human hemopexin). Serial UV-VIS spectra were recorded (350-650 nm) using a Cary 60 UV-VIS Spectrophotometer (Agilent Technologies) in order to follow the transition of heme-albumin to heme-Hpx over time. For each time-point, the concentrations of heme-albumin and heme-Hpx in the reaction mixtures were resolved by deconvolution of the full spectrum by applying Lawson-Hanson's Non Negative Least Squares algorithm of SciPy (www.scipy.org). Rates (fast and slow) of heme loss from met-Hb were calculated by fitting the following biexponential model to the data by nonlinear regression using R (r-project.org):

$$[ heme - albumin ] = \frac{{[ heme - albumin ]}_{0}}{2} \cdot ( {e}^{- {k}_{1} \cdot t} + {e}^{- {k}_{2} \cdot t} )$$

### B.12 Binding to CD163 Clearance Receptor by BLI

Streptavidin pre-coated biosensors (Cat No: 18-5019, FortéBio) were used. Biotinylated human CD163 receptor was immobilized in assay buffer (PBS, 0.01% BSA, 0.002% Tween20) at a concentration as indicated in each experiment. Hp:hemoglobin complex was diluted in assay buffer (10 mM HEPES, 150 mM NaCl, 3 mM EDTA, 25 mM CaCl₂, 0.05%, Tween 20, 0.1% BSA). The association and dissociation kinetics of haptoglobin:hemoglobin, were performed at concentrations as indicated in each experiment. The settings for each binding step were chosen as shown in Table. A reference control was included in every experiment (sensor loaded with ligand without analyte). Data was acquired on an OctetRED96 (FortéBio) at 30° C. with the following settings Table 3:

TABLE 3
Experimental OctetRED96 settings for kinetic assessment

| Step | Time (s) | Shake speed (rpm) |
|---|---|---|
| Equilibration (buffer) | 60 | 1000 |
| Loading (CD163) | 1200^{§} | 1000 |
| 2x Baseline (buffer) | 60 | 1000 |
| Association (Hp:Hb complex) | 60* or 120** | 1000 |
| Dissociation (buffer) | 600 | 1000 |

^{§}loading threshold was set to 1.5 nm
*plasma derived Hp1-1
**recombinant HuHaptoglobin2FS(148-406)-8His (SEQ ID NO: 18) and HuHemopexin-HuHaptoglobin2FS(148-406)-8His (SEQ ID NO: 18)

Data was analyzed by the Data Analysis Software (FortéBio, Version 9.0). Data was processed by performing baseline alignment to the y-axis, inter-step correction, reference sensors subtraction and curve smoothening by Savitzky-Golay Filtering. The processed kinetic dataset was globally fitted using a 1:1 binding model. The fitting accuracy was described by Chi² and R², parameters representing how well the measured results resemble those calculated from the model used to analyze the data.

### B.13 Binding to LRP1 Clearance Receptor Fragment by BLI

Streptavidin pre-coated biosensors (Cat No: 18-5019, FortéBio) were used. Biotinylated LRP1/CD91 domain 3 was immobilized at a concentration of 15 μg/mL in assay buffer (PBS, 0.1% BSA, 0.02% Tween20). Heme-Hpx complex was diluted in assay buffer (10 mM HEPES, 150 mM NaCl, 3 mM EDTA, 25 mM CaCl2, 0.05%, 0.1% BSA, Tween 20). The association and dissociation kinetics of heme-hemopexin complex, were performed at concentrations as indicated for each experiment. The settings for each binding step were chosen as shown in Table 4. A reference control was included in every experiment (sensor loaded with ligand without analyte). Data was acquired on an OctetRED96 (FortéBio) at 30° C. with the following settings:

TABLE 4
Experimental OctetRED96 settings for kinetic assessment

| Step | Time (s) | Shake speed (rpm) |
|---|---|---|
| Equilibration (buffer) | 100 | 1000 |
| Loading (LRP1-3; 15 μg/mL) | 600 | 1000 |
| Baseline (buffer) | 60 | 1000 |
| Association (heme-hpx) | 60 | 1000 |
| Dissociation (buffer) | 600 | 1000 |

Data was analyzed by the Data Analysis Software (FortéBio, Version 9.0). Data was processed by performing baseline alignment to the y-axis, inter-step correction, reference sensors subtraction and curve smoothening by Savitzky-Golay Filtering. The processed kinetic dataset was globally fitted using a 1:1 binding model. The fitting accuracy was described by Chi² and R², parameters representing how well the measured results resemble those calculated from the model used to analyze the data.

### B.14 Acceptance Criteria for BLI Experiments

For an accurate kinetic fit maximally one data point (from 7 in total) could be excluded from calculation to achieve the criteria in Table 5.

TABLE 5
Experimental OctetRED96 settings for kinetic assessment

| Criteria | Specification |
|---|---|
| R² | >0.98 |
| Chi² | <0.5 |
| Residuals | <10% |

Chi² (χ²): a measure of error between the experimental data and the fitted line
R²: indicates how well the fit and the experimental data correlate.
Residuals: Distance of each data point from fitted curve. Values should not exceed ±10% of the maximum response of the fitted curve.

### C. Results

### Example 1. Amino Acid Sequence and Processing of Wild Type Human Haptoglobin

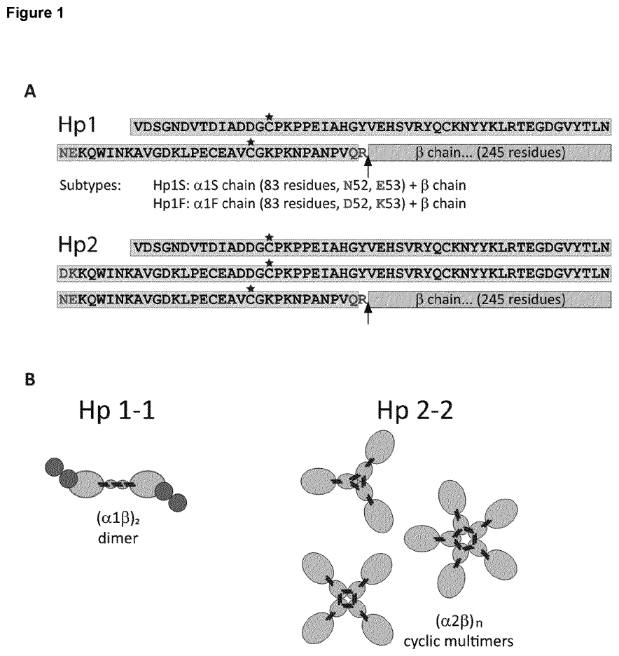

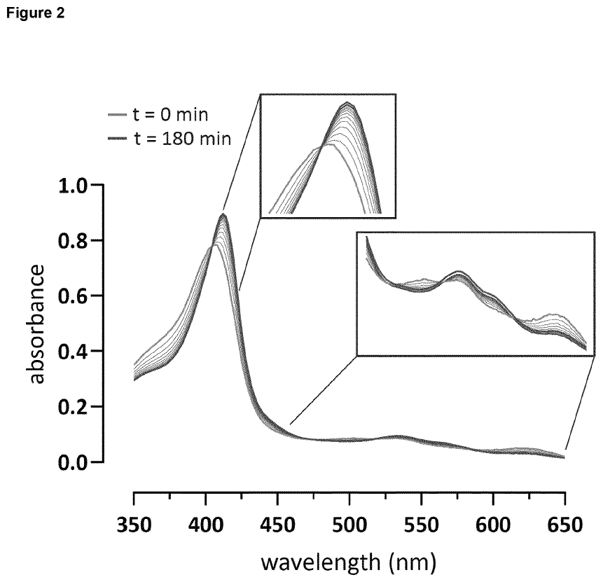

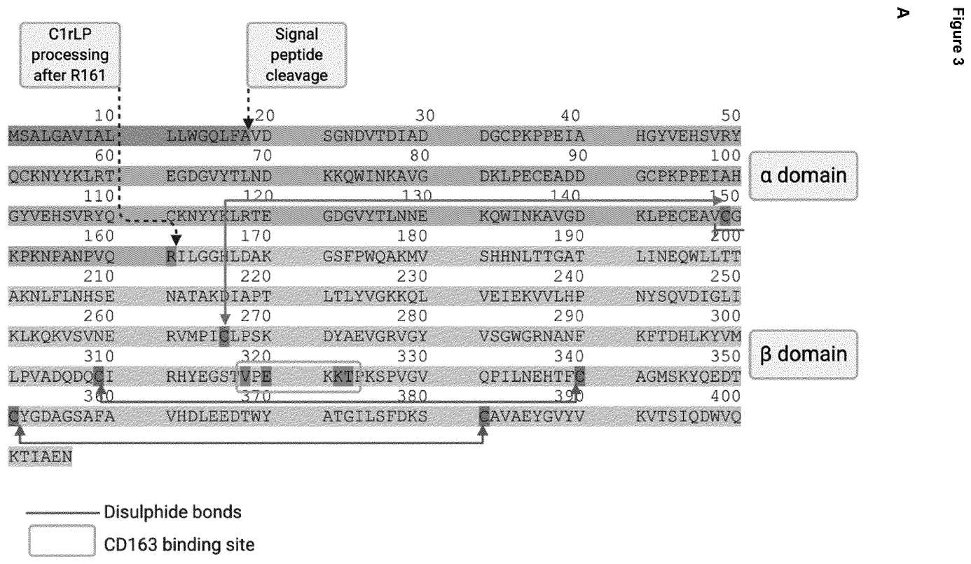

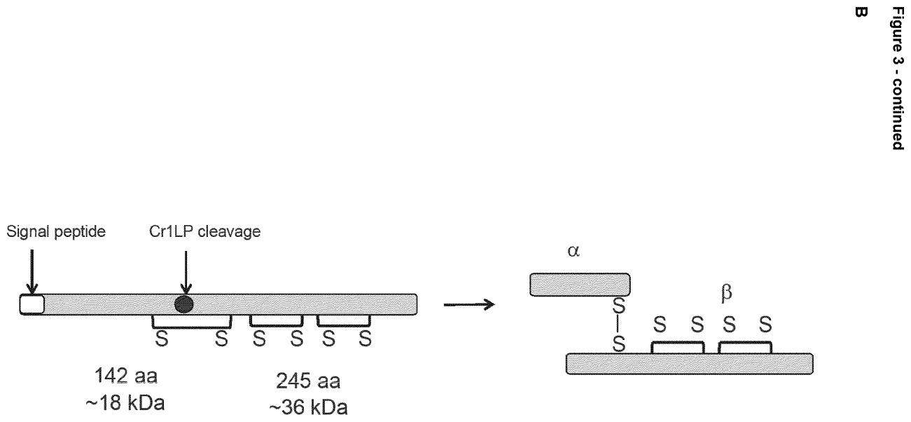

Hp was synthesized as a single polypeptide chain (pro-Hp) that is proteolytically processed in the endoplasmic reticulum by the complement C1r-like protein into α-(9 kDa) and a β-(33 kDa) subunits that are linked via disulphide bonds to form a Hp monomer. Each Hp monomeric protein can bind one Hb α-β dimer (K_{d} 10⁻¹⁵). Deoxygenated Hb does not bind Hp. In humans Hp exists in two allelic forms; Hp 1 and Hp 2, which differ only in their respective a chains i.e., the beta chain is invariant. The Hp 2 allele arose from the Hp1 allele by duplication of exons 3 and 4 (Yang F et al. 1983; PNAS; 80(219):5875-5879). The Hp1 allele can be further subdivided into Hp 1F and Hp 1S which differ by 2 amino acids in the alpha chain: Asp52Asn, Lys53Glu (van der Straten A et al. 1984, FEBS Lett. 168:103-107), which would have the numbering convention used herein as Hp 1F=D69 K70 and Hp1S=N69 E70. The structure of Hp is shown in FIG. 1.

### Example 2. Generation of the Hu-Haptoglobin Beta Chain Proteins in Mammalian Cells

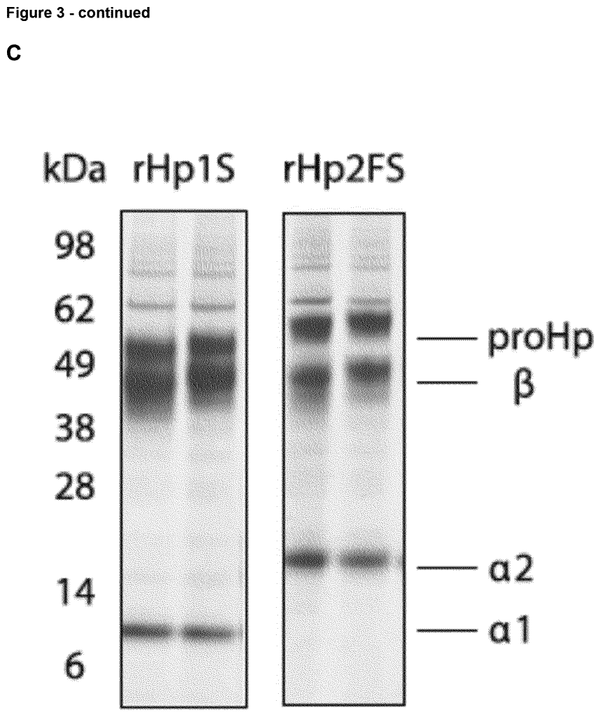

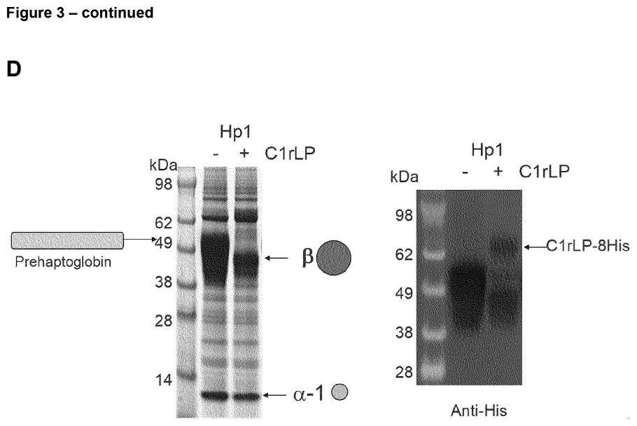

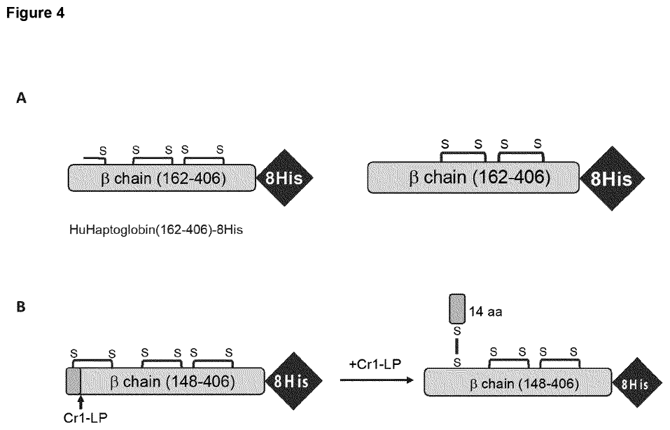

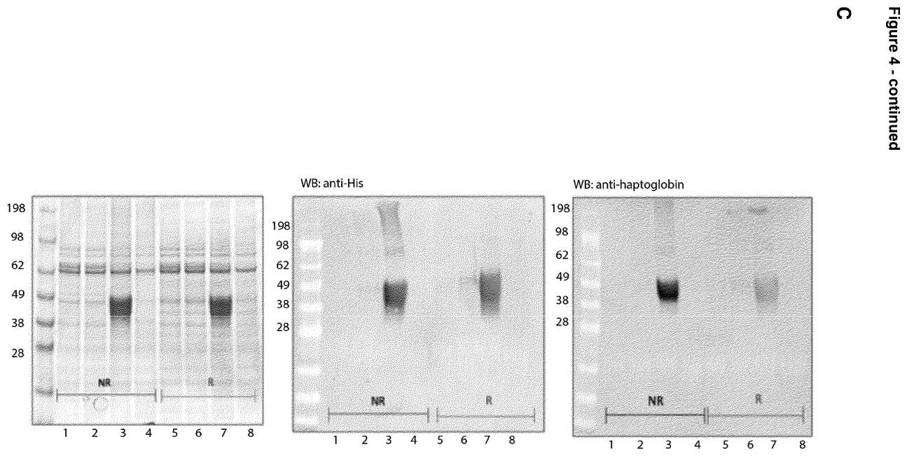

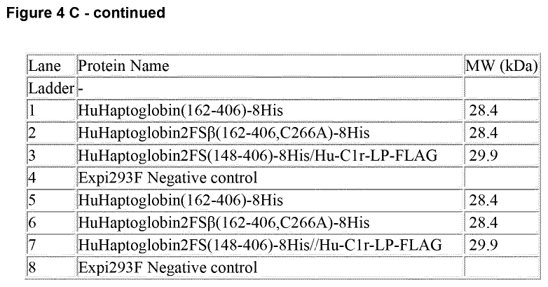

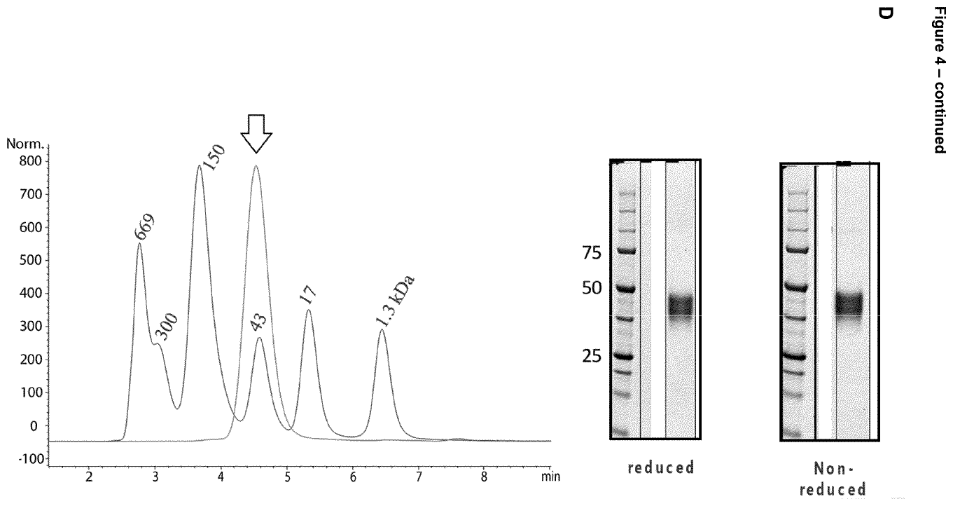

HuHaptoglobin(162-406)-8His (SEQ ID NO: 18) and HuHaptoglobin2FSβ(162-406,C266A)-8His (SEQ ID NO: 18) In order to generate the beta fragment of Hp generated in a mammalian cell, a cDNA construct was designed, HuHaptoglobin(162-406)-8His (SEQ ID NO: 18), where the beta fragment of human Hp commenced immediately after the C1rLP cleavage site in the Hp 2FS polypeptide chain at amino acid 162 (FIG. 3A, FIG. 4A). An additional variant, HuHaptoglobin2FSβ(162-406,C266A)-8His (SEQ ID NO: 18), where the unpaired cysteine at amino acid 266 was mutated to alanine (C266A, FIG. 4A) was also generated. Transient transfections of these expression constructs into Expi293F cells failed to generate any protein, indicating that the structure of the β chain had been disrupted and was therefore unstable in mammalian cells (FIG. 4B).

### HuHaptoglobin2FS(148-406)-8His (SEQ ID NO: 18)

A human Hp beta fragment construct, HuHaptoglobin2FS(148-406)-8His (SEQ ID NO: 18), encoding amino acids 148-406 that retains the C1rLP cleavage site and the cysteine required for the intra-chain disulphide bond was generated (FIG. 4B) and transfected into Expi293F cells together with a construct encoding C1rLP to allow for processing of the remaining N-terminal amino acids of the α chain. The processing was retained to allow for the generation of future proteins where a fusion partner could be placed N-terminal to the beta fragment and linked via an inter-domain disulphide bond. FIG. 4C shows that, in contrast to HuHaptoglobin(162-406)-8His (SEQ ID NO: 18) and HuHaptoglobin2FSβ(162-406,C266A)-8His (SEQ ID NO: 18), robust expression of HuHaptoglobin2FS(148-406)-8His (SEQ ID NO: 18) was observed. Size exclusion chromatography analysis using a Superdex 200 Increase 5/150 column of the purified culture supernatant (Nickel affinity chromatography combined with an additional desalting step) indicated that the protein was homogenous with no aggregation (FIG. 4D). The purity and correct processing of the protein was verified by SDS-PAGE analysis (FIG. 4D).

### Example 3. Generation of Hu-Haptoglobin Beta Chain Protein Variants with N- or C-Terminal Fusion Partners in Mammalian Cells

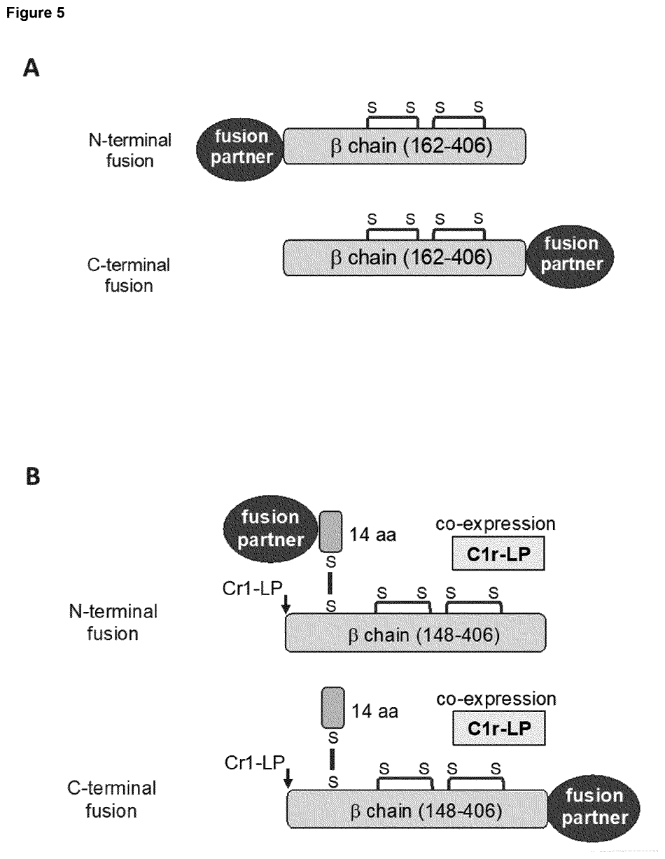

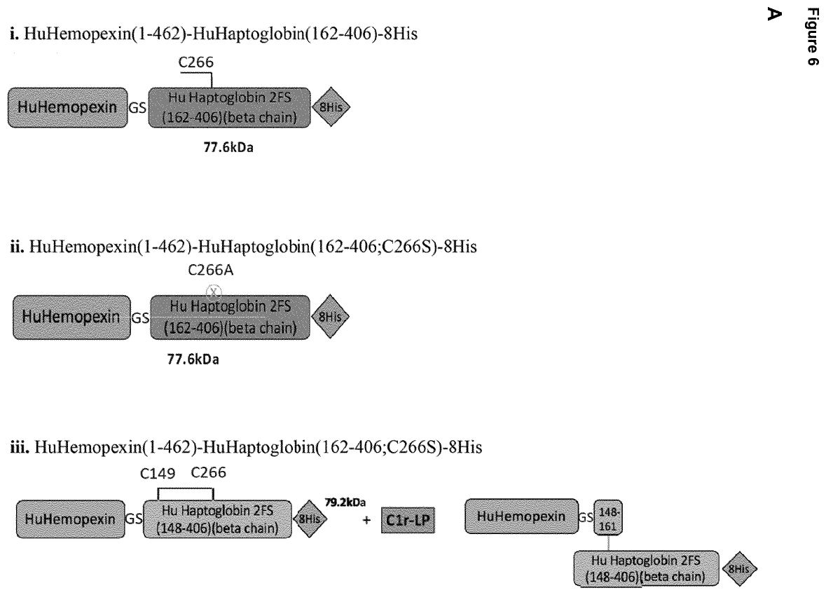

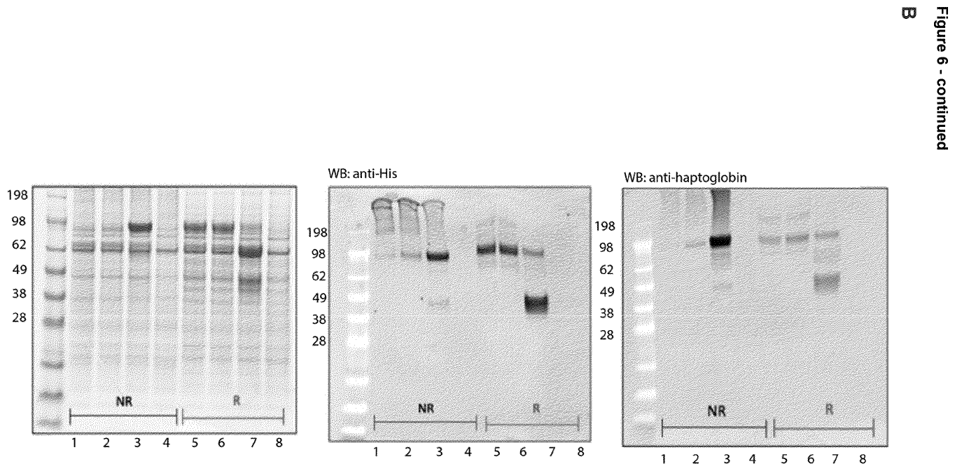

A series of proteins were generated encoding the hu-Haptoglobin beta chain (162-406 or 148-406) fused either at the N- or C-terminus with either human hemopexin (Hpx), Hpx plus mouse serum albumin (MSA), or human Hpx plus Fc, human serum albumin (HSA), mouse serum albumin (MSA), the Fc domain of mouse IgG2a or (FIGS. 5A and B). The processing was retained to allow for the generation of future proteins where a fusion partner could be placed N-terminal to the beta fragment (amino acids 148-406) and linked via an inter-domain disulphide bond.

### Generation of Hemopexin-Hu-Haptoglobin Beta Fusion Proteins

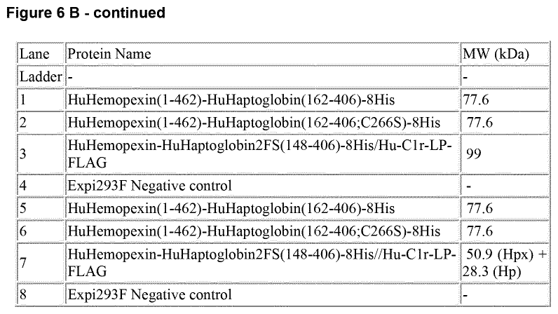

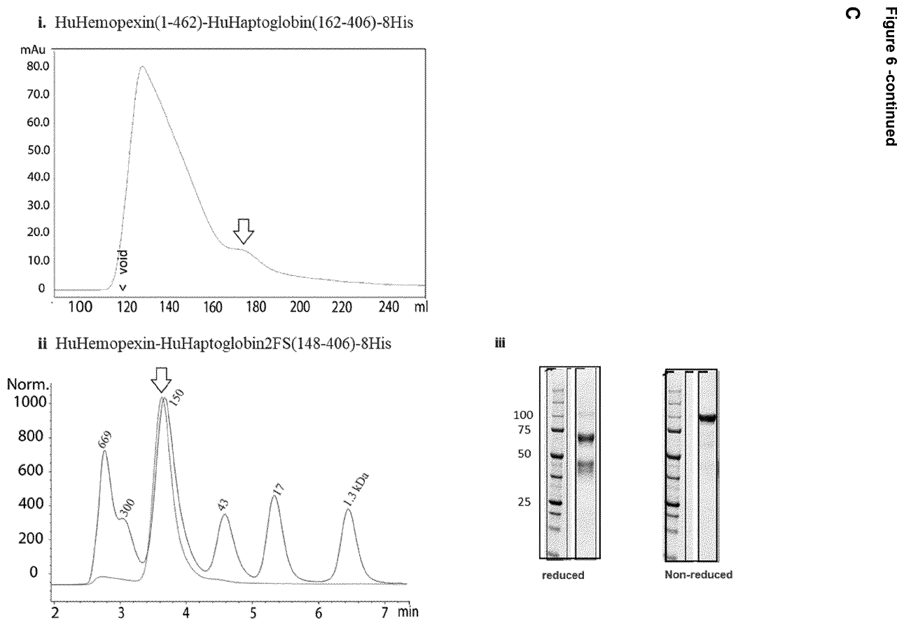

A series of constructs were generated containing human hemopexin (Hpx, amino acids 1-462; SEQ ID NO:12) at the N-terminus, followed by a Gly-Ser linker and then fused to: the human Hp beta fragment corresponding to amino acids 162-406 of SEQ ID NO:1 (FIG. 6Ai); the human Hp beta fragment corresponding to amino acids 162-406 of SEQ ID NO:1, where the unpaired cysteine at the position corresponding to amino acid residue 266 of SEQ ID NO:1 was mutated to alanine (FIG. 6Aii); and the human Hp beta fragment corresponding to amino acids 148-406 of SEQ ID NO:1 that retains the C1r-LP cleavage site (SEQ ID NO:4) and the cysteine required for intra-chain disulphide bond formation (6Aiii). Constructs containing amino acids 148-406 of haptoglobin were co-transfected into Expi293F cells in a 90:10 ratio with a construct encoding C1r-LP to enable processing at the junction of the Hp alpha chain and the Hp beta chain. FIG. 6B shows that, in contrast to Hpx constructs containing HuHaptoglobin(162-406) or HuHaptoglobin(162-406,C266A), robust expression and proteolytic cleavage at the expected site by C1r-LP of construct HuHemopexin-HuHaptoglobin2FS(148-406)-His was observed. Analytical SEC of the purified culture supernatant (nickel affinity followed by desalting) indicated that in comparison to the broad peak observed for HuHemopexin(1-462)-HuHaptoglobin(162-406)-8His (SEQ ID NO: 18) (FIG. 6Ci), HuHemopexin-HuHaptoglobin2FS(148-406)-His was produced as a homogenous protein of the expected size with dramatically reduced aggregation (FIG. 6Cii). The purity and correct processing of the protein was verified by reducing and non-reducing SDS-PAGE analysis (FIG. 6Ciii).

### Generation of HSA-Hu-Haptoglobin Beta Fusion Proteins

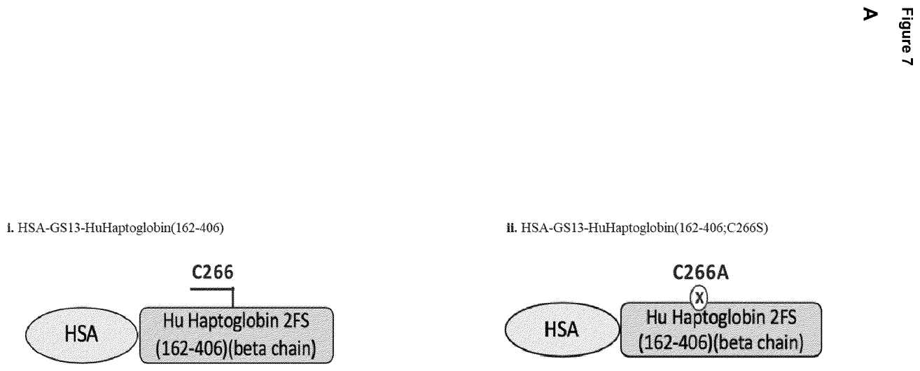

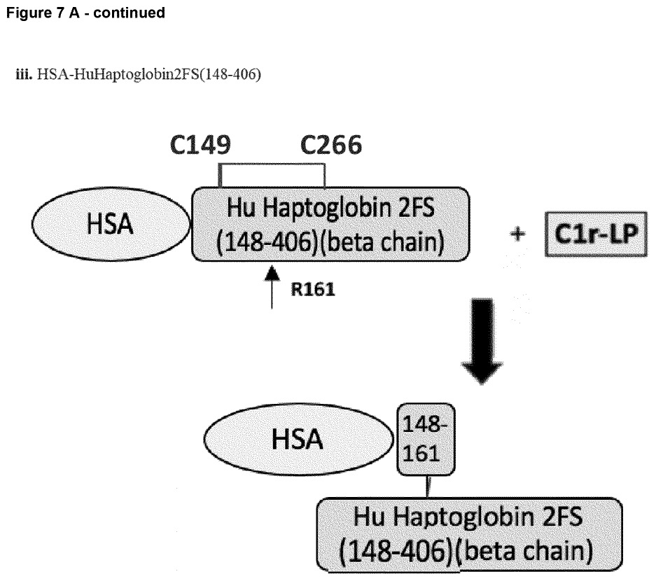

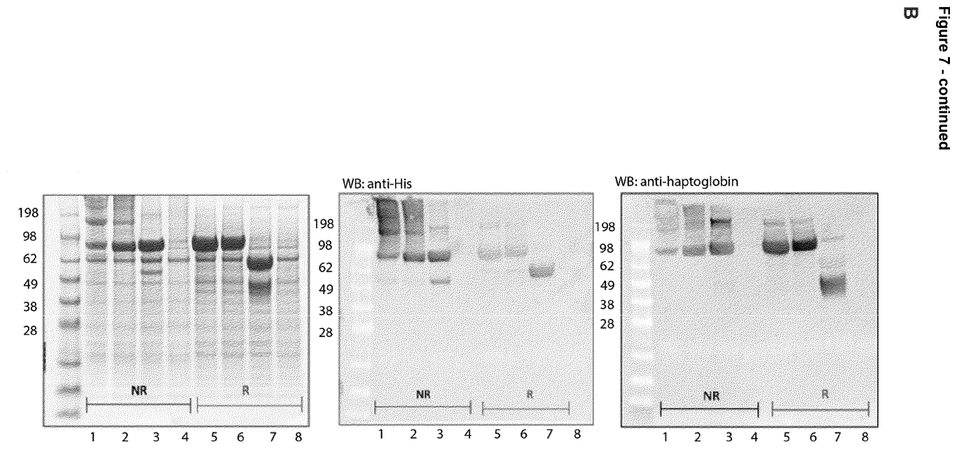

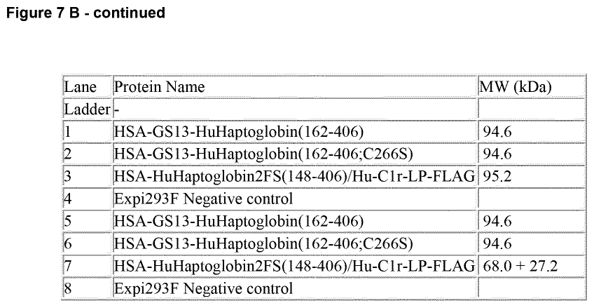

A series of constructs containing human serum albumin (HSA) at the N-terminus linker and then fused to: the human Hp beta fragment encoding amino acids 162-406 with an intervening 13×Gly-Ser linker (SEQ ID NO: 19) (FIG. 7Ai); the human Hp beta fragment encoding amino acids 162-406, where the unpaired cysteine at amino acid 266 was mutated to alanine and an intervening 13×Gly-Ser linker (SEQ ID NO: 19) (FIG. 7Aii); the human Hp beta fragment encoding amino acids 148-406 that retains the C1r-LP cleavage site and the cysteine required for the intra-chain disulphide bond was generated (FIG. 7Aiii); and transfected into Expi293F cells together with a construct encoding C1r-LP to allow for processing of the remaining N-terminal amino acids of the α chain for constructs containing this site. FIG. 7B shows that, in contrast to HSA constructs containing HuHaptoglobin(162-406) or HuHaptoglobin(162-406,C266A), robust expression and proteolytic cleavage at the expected site by C1R-LP of construct HSA-HuHaptoglobin2FS(148-406) was observed. Analytical SEC analysis using a Superdex 200 Increase 5/150 column of the purified culture supernatant (nickel affinity chromatography combined with an additional desalting step) indicated that HSA-HuHaptoglobin2FS(148-406) was homogenous with very neglible aggregation (FIG. 7Cii). In contrast, the preparative SEC chromatogram shows that there was very little HSA-GS13-HuHaptoglobin(162-406) of the expected size (as indicated by the arrow) and that the majority of the material produced was multimerised or aggregated (FIG. 7Ci). The purity and correct processing of the protein was verified by reducing and non-reducing SDS-PAGE analysis (FIG. 7Ciii).

### Generation of Fc-Hu-Haptoglobin Beta Fusion Proteins

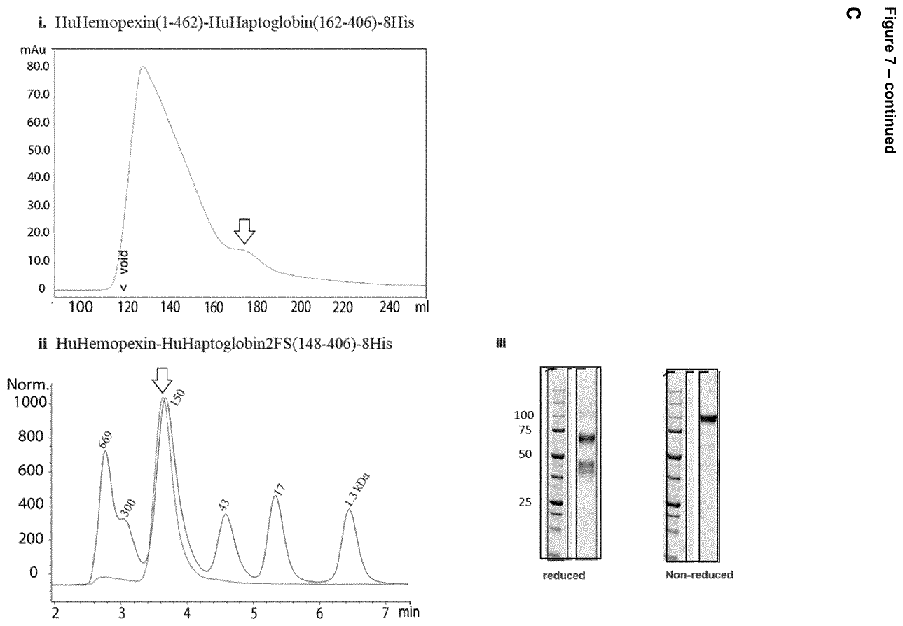

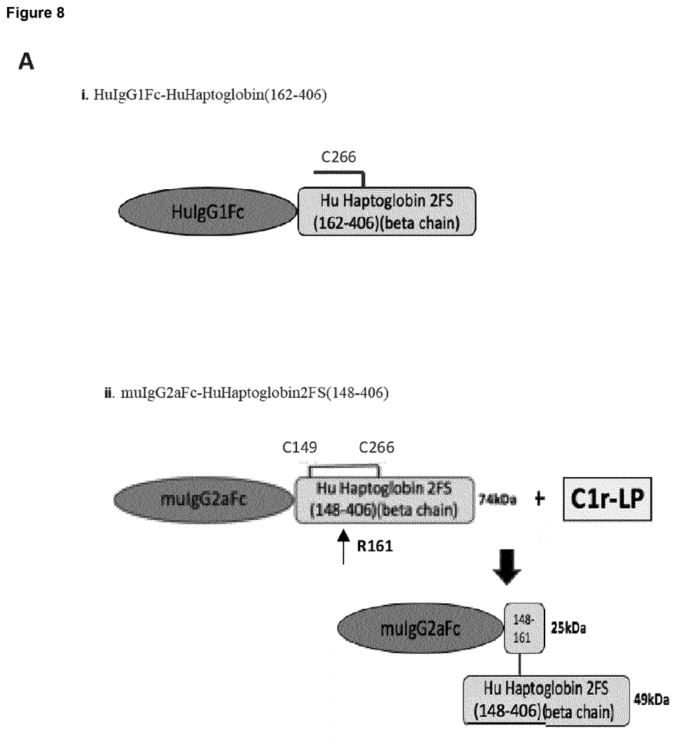

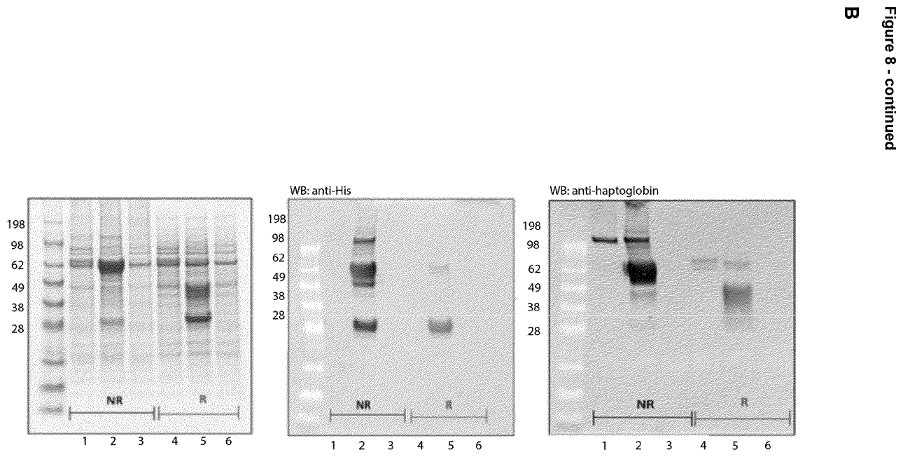

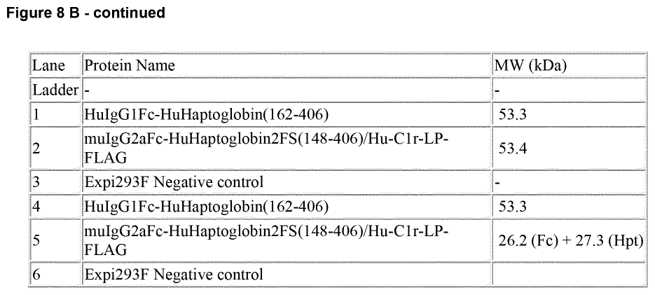

A construct containing human IgG1Fc at the N-terminus fused to the human Hp beta fragment encoding amino acids 162-406 (FIG. 8Ai); and one containing mouse IgG2aFcfused to the human Hp beta fragment encoding amino acids 148-406 that retains the C1r-LP cleavage site and the cysteine required for the intra-chain disulphide bond were generated 406 (FIG. 8Aii); and transfected into Expi293F cells together with a construct encoding C1r-LP to allow for processing of the remaining N-terminal amino acids of the α chain for constructs containing this site. FIG. 8B shows that, in contrast to the Fc construct containing HuHaptoglobin(162-406) expression and proteolytic cleavage at the expected site by C1r-LP of construct HSA-HuHaptoglobin2FS(148-406) was observed. No protein could be expressed and purified from the HuIgG1 Fc-HuHaptoglobin(162-406) construct. In contrast, muIgG2aFc-HuHaptoglobin2FS(148-406) was expressed robustly, processed correctly by C1r-LP with analytical SEC of affinity purified material revealing a dominant peak of the expected size for a Fc dimer. The fusion proteins expressed, however, were not homogenous and contained both higher and lower molecular weight species (FIG. 8Cii).

### Generation of Hemopexin-MSA-Hu-Haptoglobin Beta Fusion Proteins

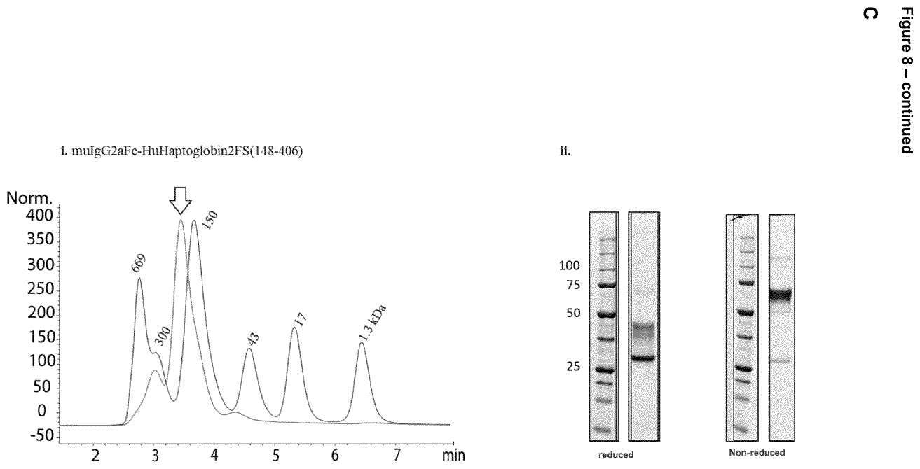

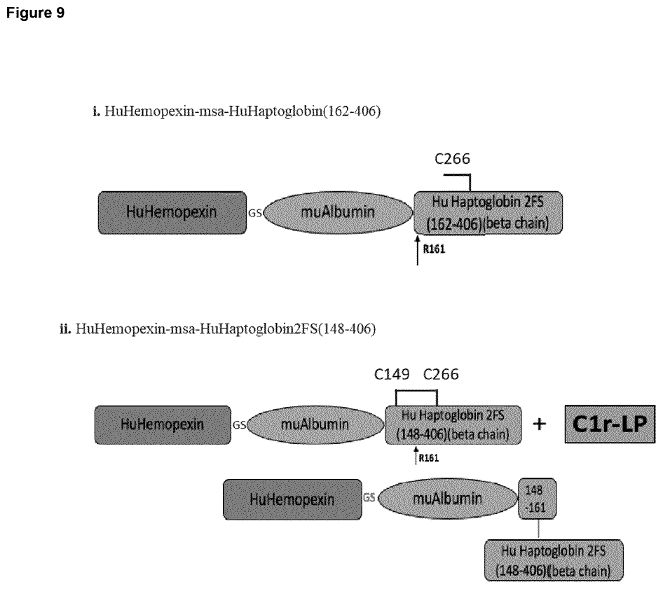

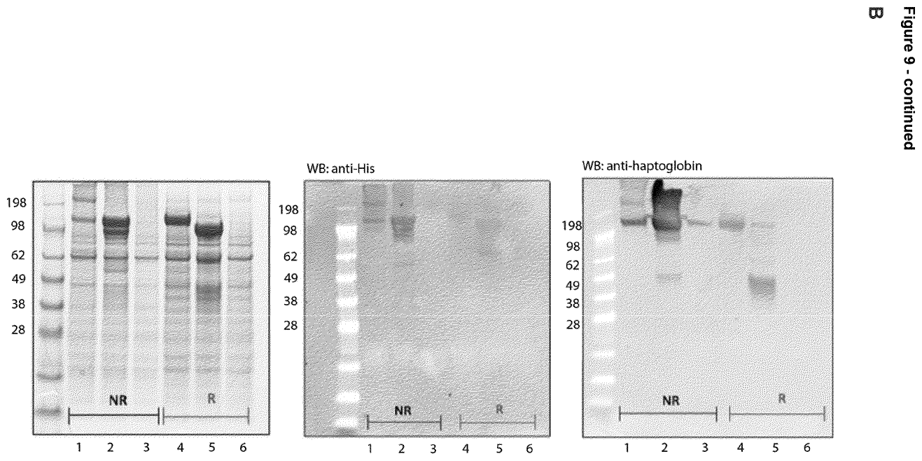

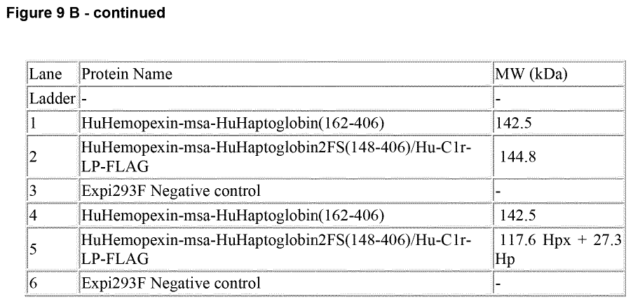

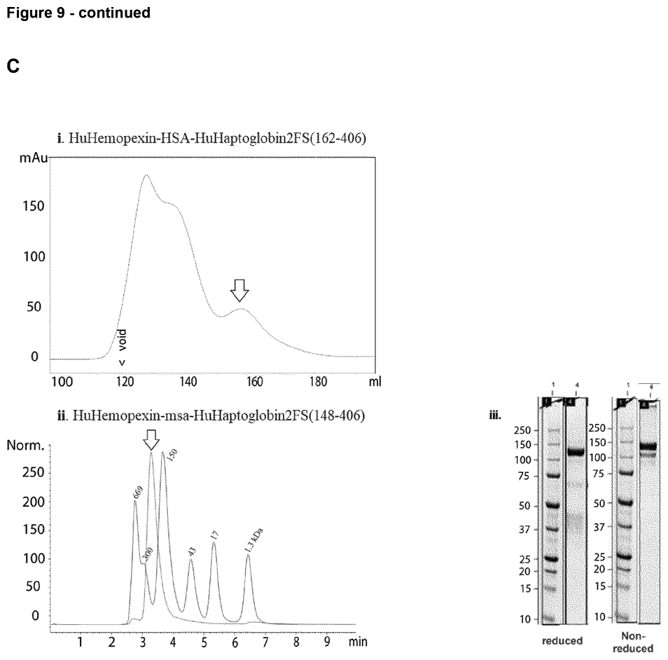

Constructs containing human hemopexin (Hpx, amino acids 1-462) at the N-terminus, followed by mouse serum albumin (MSA) and then fused to: the human Hp beta fragment encoding amino acids 162-406 (FIG. 9Ai); the human Hp beta fragment encoding amino acids 162-406 or the human Hp beta fragment encoding amino acids 148-406 that retains the C1r-LP cleavage site and the cysteine required for the intra-chain disulphide bond was generated (FIG. 9Aii); and transfected into Expi293F cells together with a construct encoding C1r-LP to allow for processing of the remaining N-terminal amino acids of the α chain for constructs containing this site. FIG. 9B shows that, in contrast to Hpx constructs containing HuHaptoglobin(162-406) robust expression and proteolytic cleavage at the expected site by C1r-LP of construct HuHemopexin-msa-HuHaptoglobin2FS(148-406) was observed. Preparative SEC of HuHemopexin-msa-HuHaptoglobin(162-406) showed it to be very low yielding with a large proportion of higher order species (FIG. 9Ci). In contrast, analytical SEC of the purified culture supernatant (Mimetic Blue affinity followed by desalting) indicated that HuHemopexin-msa-HuHaptoglobin2FS(148-406) was produced as a homogenous protein of the expected size (FIG. 9Cii). The purity and processing of the purified HuHemopexin-msa-HuHaptoglobin2FS(148-406) was verified by reducing and non-reducing SDS-PAGE analysis (FIG. 9Ciii).

### Generation of Hemopexin-Fc-Hu-Haptoglobin Beta Fusion Proteins

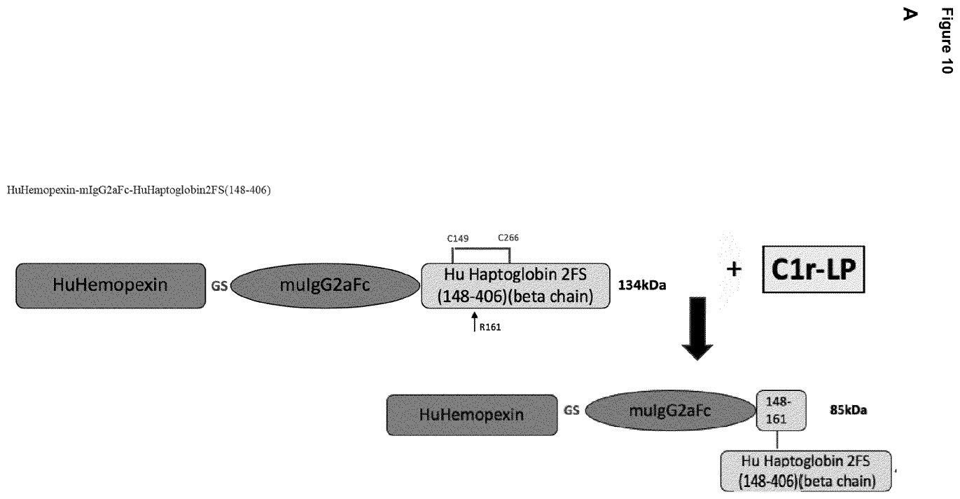

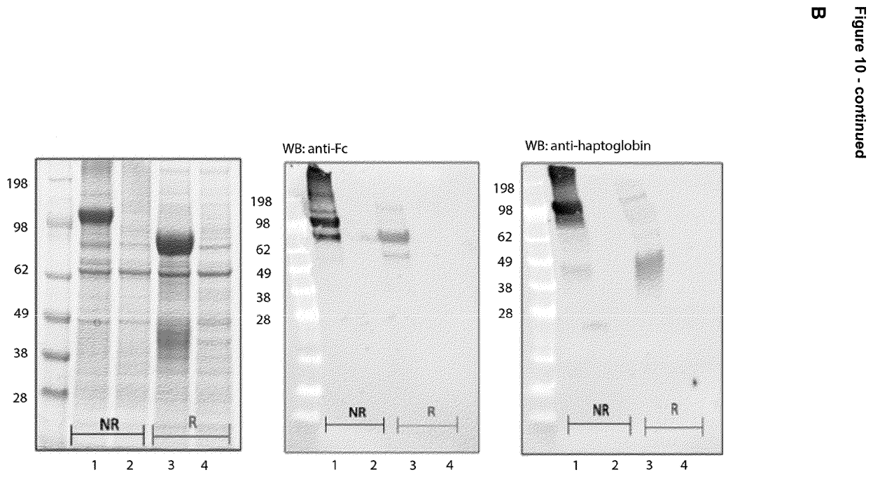

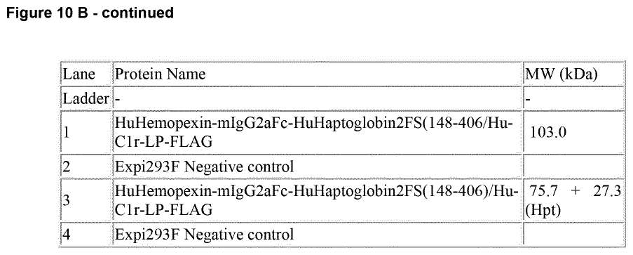

A construct containing human hemopexin (Hpx, amino acids 1-462) at the N-terminus, followed by a Gly-Ser linker, mouse IgG2aFc and then fused to the human Hp beta fragment encoding amino acids 148-406 that retains the C1r-LP cleavage site and the cysteine required for the intra-chain disulphide bond was generated (FIG. 10A) and transfected into Expi293F cells together with a construct encoding C1r-LP to allow for processing of the remaining N-terminal amino acids of the α chain for constructs containing this site. FIG. 10B shows that this constructs containing HuHaptoglobin(162-406) demonstrated high expression and proteolytic cleavage at the expected site by C1r-LP. Size exclusion chromatography analysis using a Superdex 200 Increase 5/150 column of the purified culture supernatant (MabSelect SuRe PCC affinity chromatography combined with an additional desalting step) indicated that the protein produced from HuHemopexin-mIgG2aFc-HuHaptoglobin2FS(148-406) was not homogeneous and contained a large proportion of aggregate (FIG. 10Ci), which is also visible in the Western blot (FIG. 10B). The purity and processing of the purified HuHemopexin-mIgG2aFc-HuHaptoglobin2FS(148-406) verified by reducing and non-reducing SDS-PAGE analysis (FIG. 10Cii).

### Example 4. Measurement of Hemoglobin Binding

Variants encoding Hp beta fragment amino acids 148-406 were assessed for hemoglobin binding (due to poor expression and protein aggregation none of the variants containing the wild type beta fragment (amino acid residues 162-406 of SEQ ID NO:1) were tested in terms of hemoglobin binding).

### Qualitative Hemoglobin Binding by SEC

The following variants were analysed in terms of hemoglobin binding ability in a qualitative manner: HuHaptoglobin2FS(148-406)-8His (SEQ ID NO: 18); HuHemopexin-HuHaptoglobin2FS(148-406)-8His (SEQ ID NO: 18); HSA-HuHaptoglobin2FS(148-406)-8His (SEQ ID NO: 18); muIgG2aFc-HuHaptoglobin2FS(148-406)-8His (SEQ ID NO: 18); HuHemopexin-msa-HuHaptoglobin2FS(148-406)-8His (SEQ ID NO: 18); and HuHemopexin-mIgG2aFc-HuHaptoglobin2FS(148-406)-His.

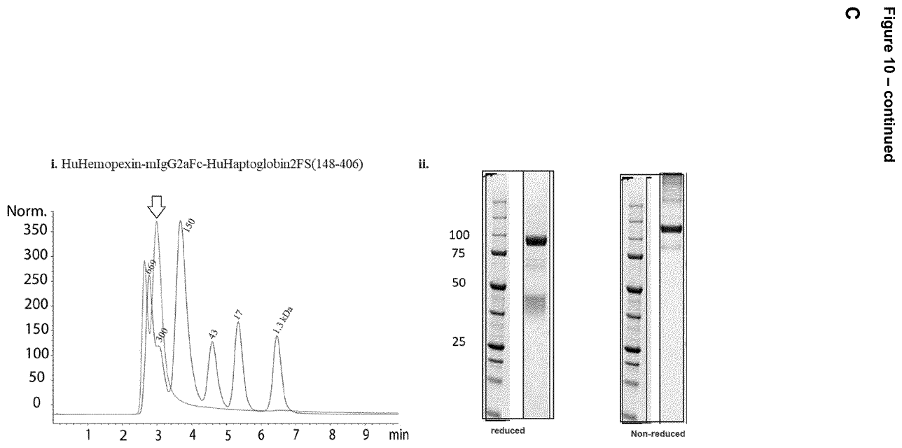

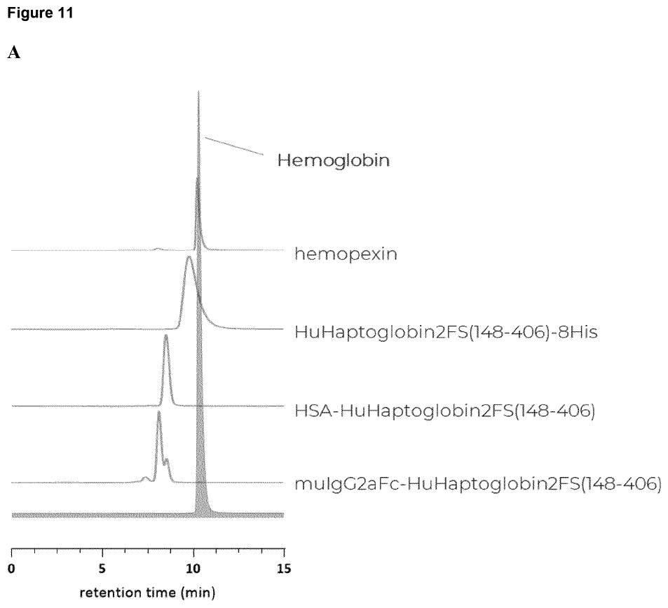

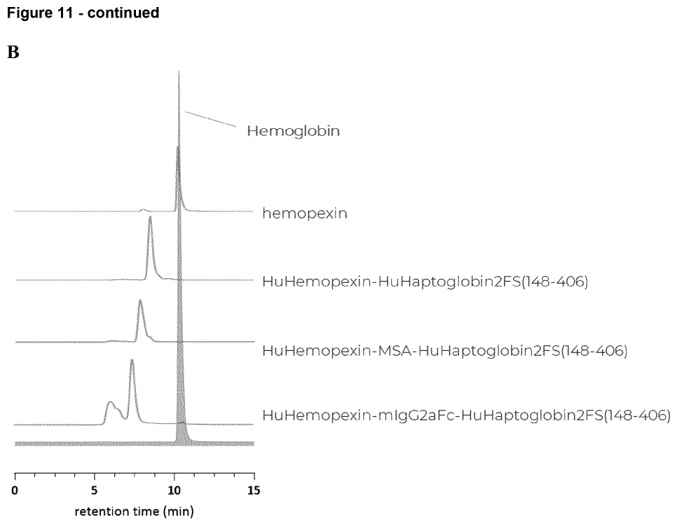

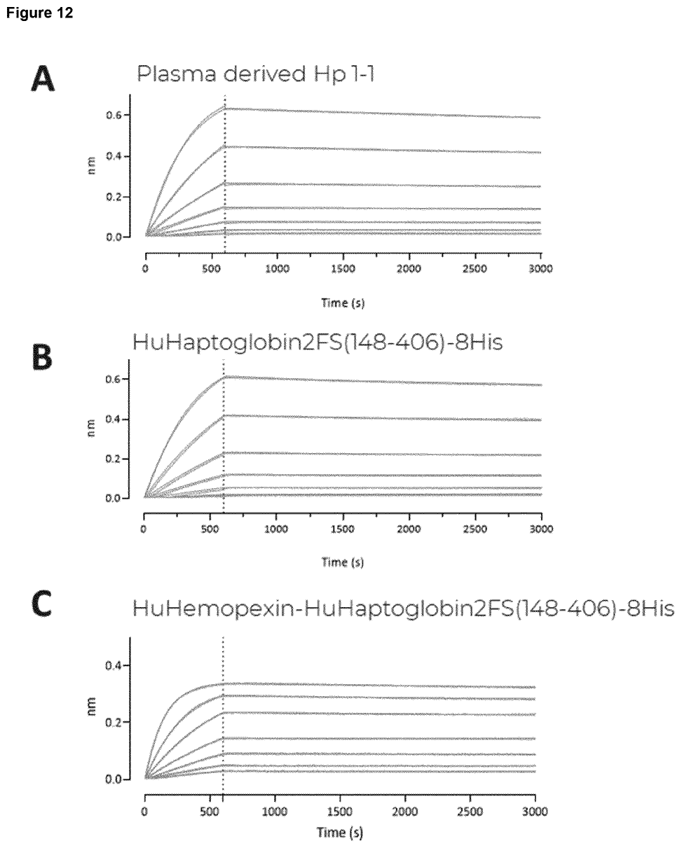

In a first step, all above recombinant variants were qualitatively analysed in terms of Hb binding. Briefly, recombinant variants were incubated with human hemoglobin at different concentration for 1 hour at 37° C. The samples were separated on a SEC column (Diol-300; 3 μm, 300×8.0 mm) and the absorbance at 405 nm was recorded. As shown in FIG. 11 HPLC traces of hemoglobin (blue lines), once incubated with a Hb binding variant, were shifted to the left indicating an increased size compared to the HPLC trace for Hb alone (red line). Based on this qualitative binding assessment, all Hp beta fragments (148-406) are able to bind hemoglobin independent of the fusion protein.

### Quantitative Hemoglobin Binding by BLI

In humans, plasma haptoglobin (Hp) binds hemoglobin with high affinity. For quantitative evaluation, the Hp mediated Hb binding was determined for the following Hp variants and the data was compared with human plasma derived Hp1-1: HuHaptoglobin2FS(148-406)-8His (SEQ ID NO: 18); HuHemopexin-HuHaptoglobin2FS(148-406)-8His (SEQ ID NO: 18); and HuHaptoglobin 1-1 (plasma derived).

Biotinylated variants were immobilized on streptavidin coated biosensors and Hb binding was assessed. As shown in Table 6, binding to human Hp1-1 (plasma derived) results in a high affinity interaction (144 μM±39). Identical binding behaviour was observed with only the Hp beta fragment (HuHaptoglobin2FS(148-406)-8His (SEQ ID NO: 18)) with a KD of 188 μM±42. Interestingly, immobilized bi-functional superscavenger (HuHemopexin-HuHaptoglobin2FS(148-406)-8His (SEQ ID NO: 18)) showed an increased binding affinity attributed by almost 2 times faster on rate (kₒₙ 4.4 10⁵ 1/Ms) and slightly slower off rate (k_{off} 1.9⁻⁵ 1/s) compared to the other two variants tested.

TABLE 6
Kinetic rates and fitting parameters (global
fit, 1:1 binding model) - Hp variants

| | Hb | K_{D} | K_{D} Error | | |
|---|---|---|---|---|---|
| Variant | [nM] | [pM] | [pM] | kₒₙ(1/Ms) | k_{off}(1/s) |
| HuHaptoglobin | 15-0.23 | 144.0 | 39.0 | 2.09 × 10⁵ | 2.92 × 10⁻⁵ |
| 1-1 (plasma | | | | | |
| derived) | | | | | |
| HuHaptoglobin2 | 15-0.23 | 188.0 | 42.0 | 1.56 × 10⁵ | 2.94 × 10⁻⁵ |
| FS(148-406)- | | | | | |
| 8His (SEQ ID | | | | | |
| NO: 18) | | | | | |
| HuHemopexin- | | | | | |
| HuHaptoglobin2 | 15-0.23 | 43.8 | 9.6 | 4.42 × 10⁵ | 1.92 × 10⁻⁵ |
| FS(148-406)- | | | | | |
| 8His (SEQ ID | | | | | |
| NO: 18) | | | | | |

### Example 5. Measurement of Heme Binding

Variants containing a hemopexin domain (heme binding domain) were assessed for their heme binding potential and compared to wild type hemopexin (plasma derived hemopexin). Briefly and as described above each variant was incubated with heme-albumin, acting as a heme donor with lower affinity as hemopexin. Spectra was recorded continuously over a time period of five hours and data was deconvoluted against reference spectra consisting of heme-albumin and hemopexin:heme. Data was fitted with a biexponential model (described in methods) using R Studio and plotted as shown in FIG. 13. Table 7 summarizes the heme binding capacity of the three variants tested in comparison to plasma derived hemopexin. All 3 variants seem to bind heme transferred from heme-albumin as indicated by the red curve, which shows the concentration of heme bound to hemopexin at the timepoint indicated. The binding to heme is described as a biexponential function due to a very rapid binding within the first few minutes followed by a much slower binding behaviour around saturation. This is the case for all variants and very comparable to plasma derived hemopexin. In terms of activity, interestingly, all variants bind around hundred percent except the fusion protein containing a mIg2aFc (around 80% activity). Rate constants are summarized in Table.

TABLE 7
Heme transferred to Hp variants containing heme
binding site and rate constants obtained from biexponential
fits of recorded absorbance signals

| | Transferred | Activity | k₁ | k₂ |
|---|---|---|---|---|
| Hp variant | heme [μM] | (%) | [min⁻¹] | [min⁻¹] |
| Plasma derived hemopexin | 5.09 | 101.9 | 0.237 | 0.008 |
| HuHemopexin- | 5.67 | 113.4 | 0.171 | 0.004 |
| HuHaptoglobin2FS(148- | | | | |
| 406) | | | | |
| HuHemopexin-msa- | 4.22 | 105.5 | 0.314 | 0.007 |
| HuHaptoglobin2FS(148- | | | | |
| 406) | | | | |
| HuHemopexin-mIgG2aFc- | 4.03 | 80.6 | 0.335 | 0.007 |
| HuHaptoglobin2FS(148- | | | | |
| 406) | | | | |

### Example 6. Measurement of Binding to CD163

Human CD163 (Hb scavenger receptor) is a 130 kDa glycoprotein almost exclusively expressed in cells of the monocyte lineage, with the highest expression detected in mature tissue macrophages, including Kupffer cells and red pulp macrophages. Structurally, CD163 belongs to the scavenger receptor cysteine-rich (SRCR) family of proteins characterized by the presence of SRCR domains in the extracellular region. CD163 is the natural high affinity scavenger receptor for the hemoglobin-haptoglobin complex and mainly expressed on monocytes and macrophages at high levels and therefore also used as a marker of cells from the monocyte/macrophage lineage. Under normal physiologic conditions, Hp counteracts the Hb toxicity by capturing the released Hb and directing it to CD163-expressing macrophages, which internalize the complex.

As a consequence of very similar Hb binding behaviour in all Hp variants tested, complexes with hemolgobin were generated and their ability to bind immobilized CD163 receptors in comparison with haptoglobin 1-1 (plasma derived):hemoglobin complex was investigated.

The following variants, complexed with human hemoglobin, were analysed in terms of CD163 receptor binding ability: HuHaptoglobin2FS(148-406)-8His (SEQ ID NO: 18); HuHemopexin-HuHaptoglobin2FS(148-406)-8His (SEQ ID NO: 18); and HuHaptoglobin 1-1 (plasma derived).

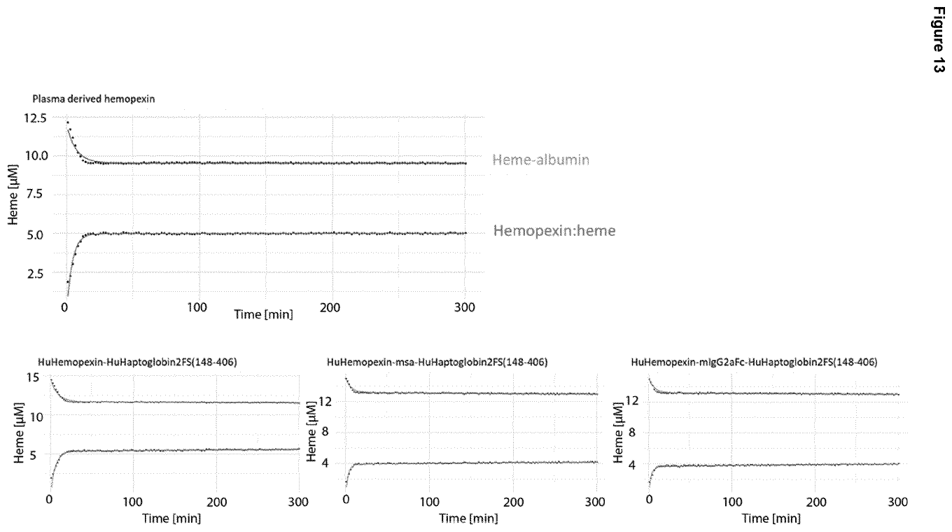

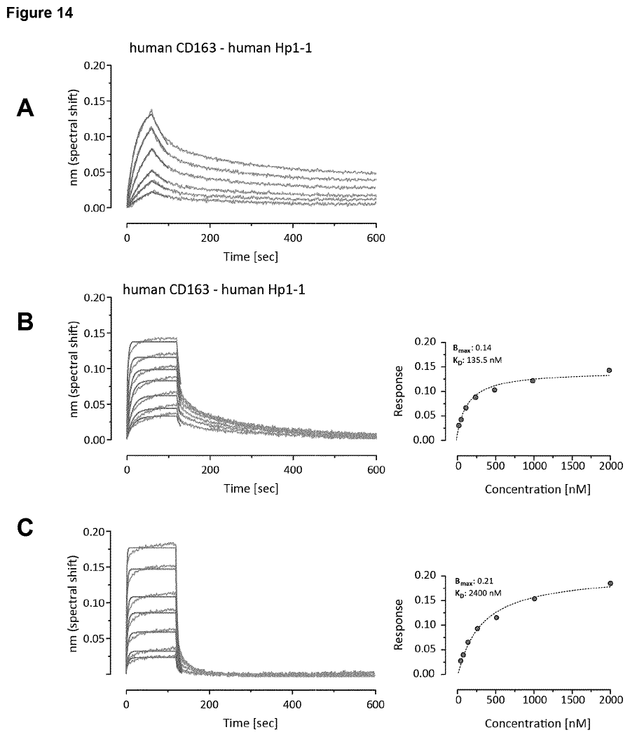

As shown in FIG. 14, both recombinant Hp variants were found to bind to immobilized CD163, although with different affinity as compared to wild type plasma derived haptoglobin 1-1:hemoglobin complexes. The binding behaviour of both recombinant variants show an increased dissociation compared to Hp1-1:Hb and determination of a proper KD was challenging by a 1:1 kinetic fit model. Therefore, a steady state analysis was performed to estimate the kinetic constant (KD). As summarized in the Table 8 below, HuHaptoglobin2FS(148-406) and HuHemopexin-HuHaptoglobin2FS(148-406), both complexed to hemoglobin have an approximately 7 times and 50 times lower affinity compared to haptoglobin:hemoglobin complexes generated with Hp1-1. This observation can potentially be explained by the presence of only one binding site (within the beta chain of Hp) compared to Hp1-1 purified from plasma.

TABLE 8
Kinetic rates and fitting parameters (global
fit, 1:1 binding model) and Steady State

| | Complex | | K_{D} | | | |
|---|---|---|---|---|---|---|
| | (Hp:Hb) | K_{D} | Error | | | Calculation |
| Variant | [nM] | [nM] | [nM] | kₒₙ(1/Ms) | k_{off}(1/s) | method |
| HuHaptoglobin | 50-1.56 | 14.0 | 1.6 | 6.8 × 10⁵ | 9.5 × 10⁻³ | Kinetic fit |
| 1-1 (plasma | | | | | | |
| derived) | | | | | | |
| HuHaptoglobin | 2000-31.25 | 143.3 | 5.7 | n/a | n/a | Steady state |
| 2FS(148-406)- | | | | | | |
| 8His (SEQ ID | | | | | | |
| NO: 18) | | | | | | |
| HuHemopexin- | 1500-234.4 | 2400.0 | 100 | n/a | n/a | Steady state |
| HuHaptoglobin | | | | | | |
| 2FS(148-406)- | | | | | | |
| 8His (SEQ ID | | | | | | |
| NO: 18) | | | | | | |

### Example 7. Preservation of Vascular Nitric Oxide Signaling in the Presence of Hemoglobin

### A. Materials and Methods

### Vascular Function Assay

The vascular function assay was performed using fresh porcine basilar arteries obtained from a local abattoir (n=20) as described in Hugelshofer et al., 2019, J Clin Invest.; 129(12):5219-5235). Briefly, after removal of the basilar artery, it was cut into 2 mm long segments. The vascular ring segments were then mounted onto the pin of a Multi-Channel Myograph System 620 M (Danish Myo Technology) and immersed in Krebs-Henseleit-Buffer. After stretching of the vessels to reach the optimal passive pre-tension (IC1 with factor k=0.80) as previously described in Hugelshofer et al., 2020, J Vasc Res; 57:106-112). 10 μM prostaglandin F2a (PGF2α; Sigma, Buchs, Switzerland) was added as a pre-contracting agent. This was followed by aNO-dependent dilation of the vessels induced by the addition of MAHMA-NONOate (ENZO Life Sciences). For all vessels, the experiment consisted of three phases: First, a NO-dip in KHB in the absence of Hb; second, a dip after the addition of 10 μM Hb; and third, a dip after the addition of an equimolar amount of haptoglobin (10 μM). The recorded dilatatory responses for each vessel were normalized to the maximum NO dilatation without Hb exposure (first dip, equal to 100%) and the level of tonic contraction before the addition of MAHMA-NONOate (equal to 0%).

Hb and Reconstituted Lipoprotein (rLP)

Hb for use in ex vivo experiments was purified from expired human blood concentrates as previously described (Elmer et al., 2011, J Chromatogr B Analyt Technol Biomed Life Sci.; 879(2): 131-138). Hb concentrations were determined by spectrophotometry with spectral deconvolution and are given as molar concentrations of total heme, which is equivalent to the single chain subunits of Hb (α- or β-chain; 1 M Hb tetramer is equivalent to 4 M heme). For the scavenger proteins (Hp, recombinant Hp-constructs and hemopexin), one mole was considered the binding capacity equivalent for one mole of heme. For all Hb used in these studies, the fraction of ferrous Hb (HbFe²⁺O₂) was always greater than 98%, as determined by spectrophotometry. Reconstituted lipoprotein (rLP) was obtained from CSL Behring, Bern, Switzerland.

### Lipoprotein Peroxidation Assay

The effect of the haptoglobin variants to prevent the oxidative Hb reactions was quantified by measuring the formation of malondialdehyde (MDA), the final product of lipid peroxidation, after incubation of Hb-haptoglobin complexes with rLP. In a 96-well plate 30 μL containing rLP (2 mg/mL) and Hb, or Hb in complex with a haptoglobin-variant (10 μM) was incubated at 37° C. for 4 hours. Subsequently, the concentration of MDA was measured using a TBARS assay (Deuel et al., 2015, Free Radical Biology and Medicine, 89:931-943). Briefly, 125 μL of 750 mM trichloroacetic acid in 1 M HCl was added to the samples, followed by vortexing (5 seconds) and the subsequent addition of 100 μL of 25 mM 2-thiobarbituric acid in 1 M NaOH. After an incubation period of 60 minutes at 80° C., the TBARS in the supernatant was quantified. To achieve more sensitive but relative quantification, the fluorescence emission was measured at 550 nm with 510 nm used as the excitation wavelength.

### B. Results

### Preservation of Vascular Nitric Oxide Signaling in Ex Vivo Vascular Function Experiments Depends on the Size and Fusion Partner of Hp

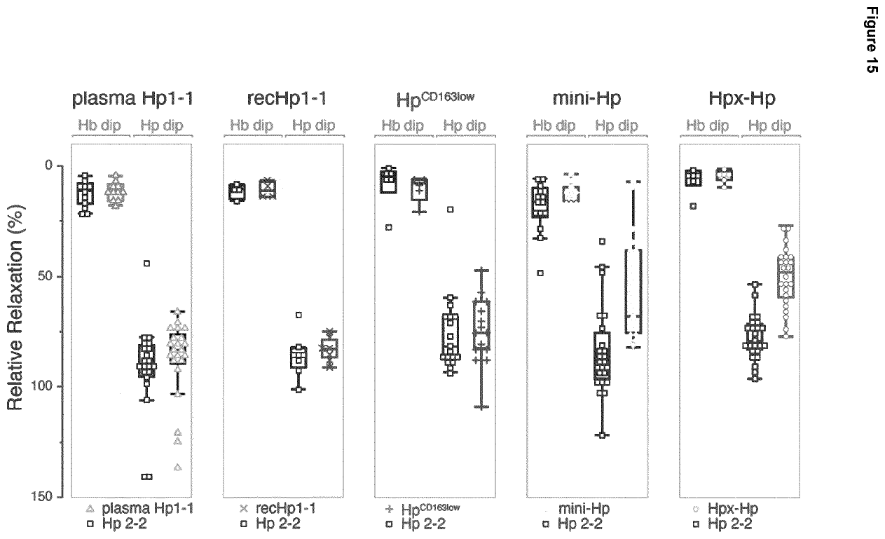

To assess the NO-sparing ability of the different Hp-variants, an established ex vivo vascular function assay was used, where the rescue to an NO-dependent vasodilatory response after addition of a Hb-scavenger was measured. In all experiments, the addition of 10 μM Hb into KHB resulted in a suppression of the vascular relaxation below 10% of the control dip without Hb (median relative relaxation: 9.7%). The subsequent addition of the Hb scavengers resulted in a clear recovery of vasodilation upon the NO-donor for all Hp-variants (FIG. 15). In all experiments, human plasma-derived Hp2-2 was used as a benchmark/gold-standard for comparison. In the groups with plasma Hp1-1, recHp1-1, and recHpCD163low, the rescue was similar compared to Hp2-2 without evidence for a difference (Table 9). For the miniHp with a smaller molecular weight, the rescue was less pronounced than with Hp2-2 (median Hp2-2: 87.93%, median miniHp 67.99%, p-value <0.0001). the bifunctional construct (SuperScavenger, Hpx-Hp-construct) showed an intermediate rescue (median 48.13%). This is likely related to the size of the Hb(αβ)₁-Hp-Hpx complex, which is smaller than the Hb(αβ)₂ Hp 1-1 complex, but larger than the Hb(a3)1.miniHp complex.

TABLE 9
Summary of the vascular function experiments comparing
the rescue of the NO-response after the addition of
different Hp-variants to our benchmark Hp2-2.
A two sided Wilcoxon signed-rank test was used to compare
between Hp2-2 and the test compound (i.e. Hp-variant).
median relative relaxation

| Hp-variant | Hp2-2 | Hp-variant | p-value |
|---|---|---|---|
| plasmaHp1-1 | 90.21% | 83.18% | 7.64E−02 |
| recHp1-1 | 86.13% | 82.95% | 2.70E−01 |
| Hp^{CD163low} | 81.68% | 75.40% | 3.76E−01 |
| miniHp | 87.93% | 67.99% | 7.19E−05 |
| superScavenger | 78.93% | 48.13% | 1.10E−07 |

### Prevention of Lipid Peroxidation is Independent of the Hp Variant

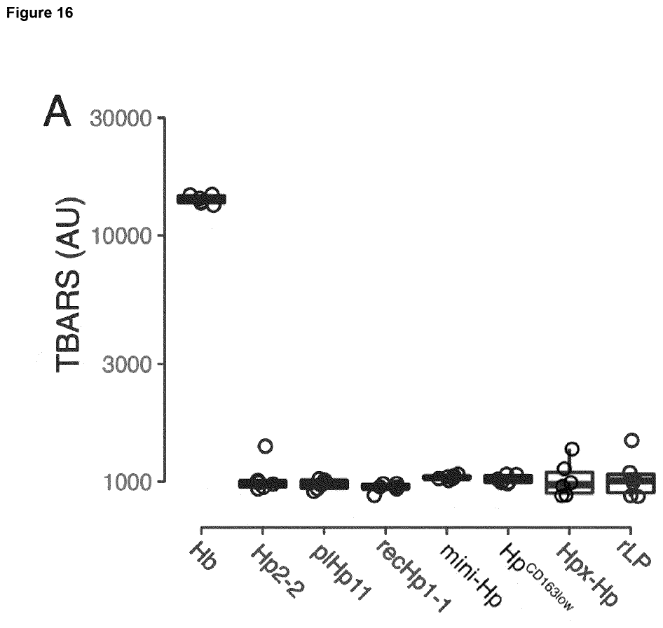

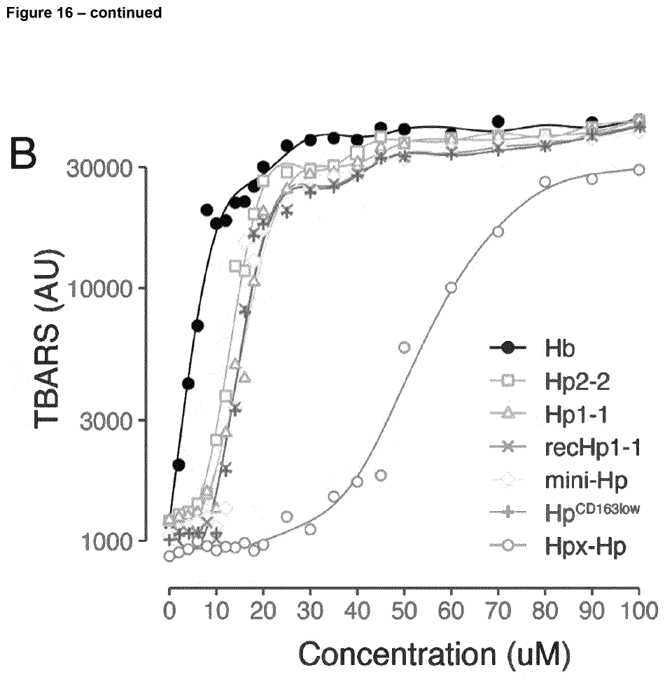

To evaluate the antioxidant potential of the recombinant haptoglobin-variants, we measured the generation of MDA in a mixture of Hb and rLP containing unsaturated phosphatidylcholine, which is the main physiological lipid substrate for Hb peroxidation reactions in vivo (Deuel et al. 2015; Free Radic Biol Med.;89:931-43). No MDA was detectable after incubation over 4 hours at 37° C. when Hb was mixed with an equimolar amount of Hp, regardless of the Hp-variant evaluated (FIG. 16A). We then repeated the experiment with increasing concentrations of Hb, ranging from sub-stoichiometric up to supra-stoichiometric concentrations in relation to Hp (FIG. 16B). In this experiment we found that recombinant Hp-variants prevent lipid peroxidation up to a Hb concentration equimolar to the Hpβ-chain. At excess concentrations of Hb over Hp, the concentration-oxidation relationship followed an identical shape as Hb alone. The only exception was observed with the Hp-Hpx superScavenger, which showed significantly lower MDA generation even at supra-stoichiometric Hb concentrations. This observation was consistent with the heme-directed antioxidant function of Hpx providing synergistic protection with Hp (Deuel et al. 2015).

LRP1 Binding of Heme:Hx Complexes with Plasma Derived Hx and Hx-Hp Fusion Protein

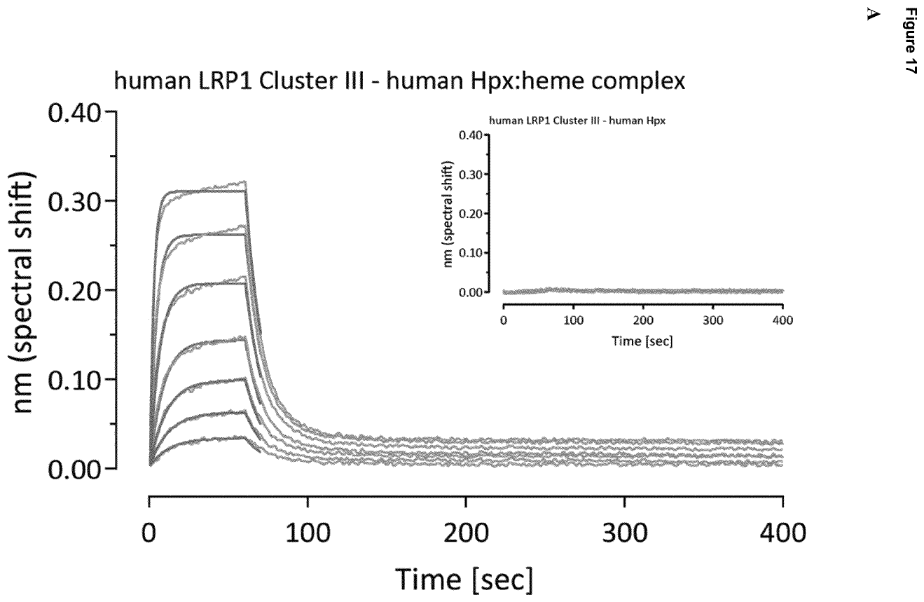

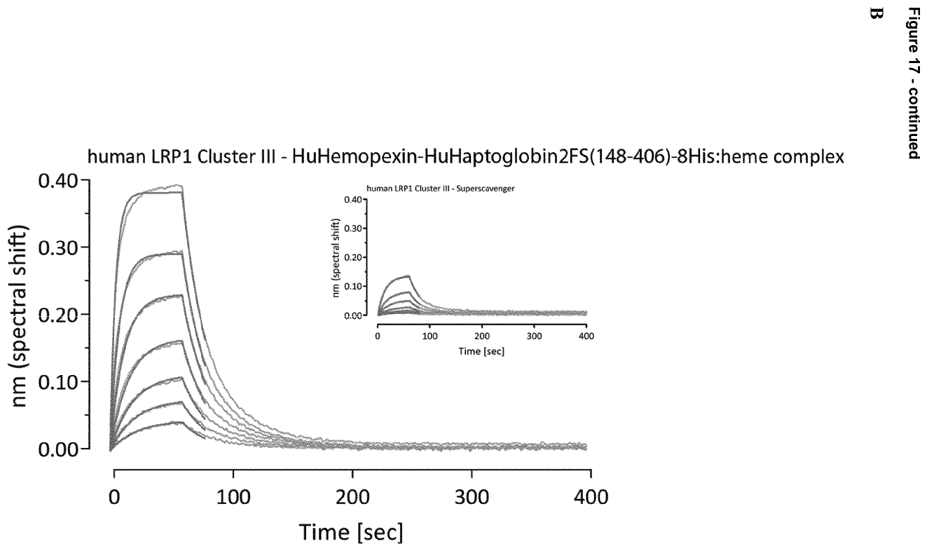

Complex formation of Hx with heme leads to association to its scavenging receptor CD91/LRP1 (Hvidberg et al. 2005 Blood 106(7):2572-9). Therefore and in a last set of binding experiments, we investigated the ability of the Hx-Hp fusion protein to bind to CD91/LRP1 in comparison to heme complexed plasma derived Hx. Since the full length receptor is challenging to express recombiantly, we immobilized only a fragment of CD91/LRP1 (cluster III). In previous work we identified that of the four clusters, cluster Ill contains the binding site for heme:Hx (unpublished data). We found that both heme complexes bound the immobilized receptor fragment in a very comparable manner with a KD in the high nano molar range as shown in Table 10. In contrast to Hx, which does not bind in absence of heme to LRP1, as illustrated in FIG. 17, uncomplexed Hx-Hp did show a very low affinity binding to immobilized LRP1. Whether this is due to the artificial scaffold of the fusion protein remains to be investigated.

TABLE 10
Kinetic rates and fitting parameters (global fit, 1:1 binding model)

| | Complex | | K_{D} | | | |
|---|---|---|---|---|---|---|
| | (Hb:Hp) | K_{D} | Error | | | Calculation |
| Variant | [nM] | [nM] | [nM] | kₒₙ(1/Ms) | k_{off}(1/s) | method |
| heme:Hx | 2000-31.25 | 392.2 | 22.5 | 1.7 × 10⁵ | 6.8 × 10⁻² | Kinetic fit |
| heme:Hx-Hp | 2000-31.25 | 479.6 | 5.9 | 9.1 × 10⁴ | 4.4 × 10⁻² | Kinetic fit |
| Hx-Hp | 2000-31.25 | 2700.5 | 1057.1 | 1.5 × 10⁴ | 3.9 × 10⁻² | Kinetic fit |

### Discussion

The present inventors have previously shown that Hp can be expressed at high yield as functional protein in a transient eukaryotic expression system yielding a molecule of the appropriate size, structure and function (Schaer, Owczarek et al. 2018, BMC Biotechnol.; 18:15).

The major functions of Hp—binding of Hb and binding to CD163—are mediated by the Hp β-chain (see Melamed-Frank 2001 Blood; 98(13):3693-8 and Alayash, Andersen et al. 2013; In: Trends in Biotechnology, 31(1):2-3). In order to further characterise the minimal domain of haptoglobin required for Hb binding, a construct was generated where the β-chain of human Hp commenced immediately after the C1r-LP cleavage site in the Hp polypeptide chain. An additional variant, where an unpaired cysteine at amino acid 266 was mutated to alanine, was also generated. However, transient transfections of these expression constructs failed to generate any protein, indicating that the structure of the R-chain had been disrupted and was therefore unstable in mammalian cells. Unexpectedly, an alternatively designed construct, where the human Hp β-chain further comprises an additional 14 contiguous N-terminal amino acids of the Hp α-chain, and thus retaining the C1r-LP cleavage site and the cysteine required for the intra-chain disulphide bond, resulted in robust and stable expression of a functional Hp β-chain protein. The modified construct also allows for the generation of fusion proteins, where a fusion partner (e.g., an additional functional moiety) may be placed, for example, at the N-terminus of the N-terminal truncated Hp alpha chain linked to the Hp β-chain fragment via an inter-domain disulphide bond. This advantageously allows for the production of dual-targeting therapeutic molecules, an illustrative example of which includes a haptoglobin-hemopexin (Hp-Hpx) conjugates, which may be used as a scavenger of both cell-free haemoglobin and cell-free heme.

1. An expression system for producing a recombinant haptoglobin beta chain, or a haemoglobin-binding fragment thereof, in a mammalian cell, the expression system comprising:
(a) a first nucleic acid sequence encoding an N-terminal truncated pro-haptoglobin (proHp), wherein the N-terminal truncated proHp comprises (i) at least 14 contiguous C-terminal amino acid residues of a haptoglobin alpha chain and (ii) a haptoglobin beta chain, or a haemoglobin-binding fragment thereof, and wherein the N-terminal truncated proHp comprises an internal enzymatic cleavage site between the at least 14 contiguous C-terminal amino acid residues of a haptoglobin alpha chain and the haptoglobin beta chain, or haemoglobin-binding fragment thereof, and
(b) a second nucleic acid sequence encoding an enzyme capable of cleaving the N-terminal truncated proHp at the enzymatic cleavage site;
wherein, upon introduction of the first nucleic acid sequence and the second nucleic acid sequence into a mammalian cell, and subsequent expression of the N-terminal truncated proHp and the enzyme in the cell, the enzyme is capable of cleaving the N-terminal truncated proHp at the internal enzymatic cleavage site, thereby releasing the haptoglobin beta chain, or haemoglobin-binding fragment thereof, from the N-terminal truncated proHp.

2. The expression system of claim 1, wherein the proHp is a human proHp.

3. The expression system of claim 2, wherein the human proHp comprises an amino acid sequence having at least 80% sequence identity to SEQ ID NO:1.

4. The expression system of claim 2, wherein the N-terminal truncated proHp comprises an amino acid sequence having at least 80%, at least 90%, or at least 95%, or 100% sequence identity to amino acid residues 148 to 406 of SEQ ID NO:1.

5. (canceled)

6. (canceled)

7. (canceled)

8. The expression system of claim 1, wherein the internal enzymatic cleavage site is selected from the group consisting of a furin cleavage site, a serine protease cleavage site, a cysteine protease cleavage site, an aspartic protease cleavage site, a metalloprotease cleavage site, and a threonine protease cleavage site.

9. (canceled)

10. (canceled)

11. (canceled)

12. (canceled)

13. The expression system of claim 1, wherein the N-terminal truncated proHp comprises a disulphide bond between the 14 contiguous C-terminal amino acid residues of the Hp α-chain and the Hp β-chain.

14. The expression system of claim 13, wherein (a) the N-terminal truncated proHp comprises a disulphide bond between a cysteine residue within the at least 14 contiguous C-terminal amino acid residues of the haptoglobin alpha chain and at a position corresponding to amino acid position 266 of SEQ ID NO:1: or (b) the N-terminal truncated proHp comprises a disulphide bond between cysteine residues at positions corresponding to amino acid positions 149 and 266 of SEQ ID NO:1.

15. (canceled)

16. The expression system of claim 1, wherein the N-terminal truncated proHp encoded by the first nucleic acid sequence comprises an additional functional moiety optionally comprising albumin, an Fc domain of an immunoglobulin or an FcRn-binding fragment thereof, or hemopexin or a heme-binding fragment thereof.

17. (canceled)

18. (canceled)

19. (canceled)

20. (canceled)

21. (canceled)

22. (canceled)

23. (canceled)

24. The expression system of claim 18, wherein expression of the N-terminal truncated proHp in the mammalian cell is driven by a first mammalian regulatory sequence operably linked to the first nucleic acid sequence and expression of the serine protease in the mammalian cell is driven by a second mammalian regulatory sequence operably linked to the second nucleic acid sequence.

25. (canceled)

26. The expression system of claim 18, wherein expression of the polypeptide in the mammalian cell is driven by a first mammalian regulatory sequence operably linked to the first nucleic acid sequence and expression of the serine protease in the mammalian cell is driven by a second mammalian regulatory sequence operably linked to the second nucleic acid sequence.

27. (canceled)

28. An expression vector for producing a recombinant haptoglobin beta chain, or a haemoglobin-binding fragment thereof, in a mammalian cell, wherein the vector comprises:
(a) the first nucleic acid sequence according to claim 1; and
(b) the second nucleic acid sequence according to claim 1.

29. The expression vector of claim 28, wherein (a) the first nucleic acid sequence and the second nucleic acid sequence are operably linked to a common mammalian regulatory sequence, or (b) the first nucleic acid sequence is operably linked to a first mammalian regulatory sequence and the second nucleic acid sequence is operably linked to a second mammalian regulatory sequence and wherein the first mammalian regulatory sequence is different to the second mammalian regulatory sequence.

30. (canceled)

31. A mammalian cell transfected or transduced with the expression system of claim 1.

32. The mammalian cell of claim 31, wherein the cell is a Chinese hamster ovary (CHO) cell or a human embryonic kidney (HEK) cell.

33. (canceled)

34. A method of producing a recombinant haptoglobin beta chain, or a haemoglobin-binding fragment thereof, the method comprising:
(a) introducing the expression system of claim 1 into a mammalian cell to produce a transfected mammalian cell;
(b) culturing the transfected mammalian cell of step (a) under conditions and for a period of time sufficient to allow production of the recombinant haptoglobin beta chain, or the haemoglobin-binding fragment thereof by the transfected mammalian cell; and
(c) collecting the recombinant haptoglobin beta chain, or the haemoglobin-binding fragment thereof produced in step (b).

35. The method of claim 34, wherein the cell is a CHO cell or a human embryonic kidney (HEK) cell.

36. (canceled)

37. A recombinant haemoglobin-binding molecule comprising (i) a haptoglobin beta chain, or a haemoglobin-binding fragment thereof, and (ii) an N-terminal truncated haptoglobin alpha chain, wherein the N-terminal truncated haptoglobin alpha chain comprises at least 14 contiguous C-terminal amino acid residues of the haptoglobin alpha chain, wherein the at least 14 contiguous C-terminal amino acid residues of the haptoglobin alpha chain is non-contiguous to the haptoglobin beta chain, or the haemoglobin-binding fragment thereof, and wherein the N-terminal truncated haptoglobin alpha chain is attached to the haptoglobin beta chain, or the haemoglobin-binding fragment thereof.

38. The recombinant haemoglobin-binding molecule of claim 37, wherein the N-terminal truncated haptoglobin alpha chain is attached to the haptoglobin beta chain, or the haemoglobin-binding fragment thereof, by a disulphide bond between a first cysteine residue in the haptoglobin beta chain, or the haemoglobin-binding fragment thereof, and a second cysteine residue in the at least 14 contiguous C-terminal amino acid residues of the haptoglobin alpha chain.

39. The recombinant haemoglobin-binding molecule of claim 37, wherein the haemoglobin-binding molecule further comprises an additional functional moiety optionally comprising albumin, an Fc domain of an immunoglobulin or an FcRn-binding fragment thereof, or hemopexin or a heme-binding fragment thereof.

40. (canceled)

41. (canceled)

42. (canceled)

43. (canceled)

44. The recombinant haemoglobin-binding molecule of claim 37, wherein the haptoglobin beta chain comprises an amino acid sequence having at least 80%, at least 90%, at least 95% or 100% sequence identity to amino acid residues 162 to 406 of SEQ ID NO:1.

45. A recombinant haptoglobin beta chain, or a haemoglobin-binding fragment thereof, produced by the method according to claim 34.

46. A pharmaceutical composition comprising a therapeutically effective amount of the recombinant haptoglobin beta chain, or haemoglobin-binding fragment thereof, of claim 45, and a pharmaceutically acceptable carrier.

47. A method of treating or preventing a condition associated with cell-free haemoglobin (Hb) in a subject, the method comprising administering to a subject in need thereof a therapeutically effective amount of the recombinant haptoglobin beta chain, or haemoglobin-binding fragment thereof, of claim 45 for a period of time sufficient to allow the haptoglobin beta chain, or haemoglobin-binding fragment thereof, to form a complex with, and thereby neutralise, the cell-free Hb.

48. (canceled)

49. (canceled)

50. (canceled)

51. The method of claim 47, wherein the condition is a haemorrhagic stroke or a hemoglobinopathy.

52. (canceled)

53. (canceled)

54. (canceled)

55. (canceled)

56. (canceled)

57. (canceled)

58. (canceled)

59. A method of treating or preventing a condition associated with cell-free haemoglobin (Hb) in a subject, the method comprising administering to a subject in need thereof a therapeutically effective amount of the recombinant haemoglobin-binding molecule of claim 37 for a period of time sufficient to allow the haptoglobin beta chain, or haemoglobin-binding fragment thereof, to form a complex with, and thereby neutralise, the cell-free Hb.


SEQUENCE LISTING (21 sequences)

SEQ ID NO: 1 - protein, 406 aa, Homo sapiens
MSALGAVIALLLWGQLFAVDSGNDVTDIADDGCPKPPEIAHGYVEHSVRYQCKNYYKLRTEGDGVYTLNDKKQWINKAVGDKLPECEADDGCPKPPEIAHGYVEHSVRYQCKNYYKLRTEGDGVYTLNNEKQWINKAVGDKLPECEAVCGKPKNPANPVQRILGGHLDAKGSFPWQAKMVSHHNLTTGATLINEQWLLTTAKNLFLNHSENATAKDIAPTLTLYVGKKQLVEIEKVVLHPNYSQVDIGLIKLKQKVSVNERVMPICLPSKDYAEVGRVGYVSGWGRNANFKFTDHLKYVMLPVADQDQCIRHYEGSTVPEKKTPKSPVGVQPILNEHTFCAGMSKYQEDTCYGDAGSAFAVHDLEEDTWYATGILSFDKSCAVAEYGVYVKVTSIQDWVQKTIAEN

SEQ ID NO: 2 - protein, 347 aa, Homo sapiens
MSALGAVIALLLWGQLFAVDSGNDVTDIADDGCPKPPEIAHGYVEHSVRYQCKNYYKLRTEGDGVYTLNNEKQWINKAVGDKLPECEAVCGKPKNPANPVQRILGGHLDAKGSFPWQAKMVSHHNLTTGATLINEQWLLTTAKNLFLNHSENATAKDIAPTLTLYVGKKQLVEIEKVVLHPNYSQVDIGLIKLKQKVSVNERVMPICLPSKDYAEVGRVGYVSGWGRNANFKFTDHLKYVMLPVADQDQCIRHYEGSTVPEKKTPKSPVGVQPILNEHTFCAGMSKYQEDTCYGDAGSAFAVHDLEEDTWYATGILSFDKSCAVAEYGVYVKVTSIQDWVQKTIAEN

SEQ ID NO: 3 - protein, 347 aa, Homo sapiens
MSALGAVIALLLWGQLFAVDSGNDVTDIADDGCPKPPEIAHGYVEHSVRYQCKNYYKLRTEGDGVYTLNDKKQWINKAVGDKLPECEAVCGKPKNPANPVQRILGGHLDAKGSFPWQAKMVSHHNLTTGATLINEQWLLTTAKNLFLNHSENATAKDIAPTLTLYVGKKQLVEIEKVVLHPNYSQVDIGLIKLKQKVSVNERVMPICLPSKDYAEVGRVGYVSGWGRNANFKFTDHLKYVMLPVADQDQCIRHYEGSTVPEKKTPKSPVGVQPILNEHTFCAGMSKYQEDTCYGDAGSAFAVHDLEEDTWYATGILSFDKSCAVAEYGVYVKVTSIQDWVQKTIAEN

SEQ ID NO: 4 - protein, 487 aa, Homo sapiens
MPGPRVWGKYLWRSPHSKGCPGAMWWLLLWGVLQACPTRGSVLLAQELPQQLTSPGYPEPYGKGQESSTDIKAPEGFAVRLVFQDFDLEPSQDCAGDSVTISFVGSDPSQFCGQQGSPLGRPPGQREFVSSGRSLRLTFRTQPSSENKTAHLHKGFLALYQTVAVNYSQPISEASRGSEAINAPGDNPAKVQNHCQEPYYQAAAAGALTCATPGTWKDRQDGEEVLQCMPVCGRPVTPIAQNQTTLGSSRAKLGNFPWQAFTSIHGRGGGALLGDRWILTAAHTIYPKDSVSLRKNQSVNVFLGHTAIDEMLKLGNHPVHRVVVHPDYRQNESHNFSGDIALLELQHSIPLGPNVLPVCLPDNETLYRSGLLGYVSGFGMEMGWLTTELKYSRLPVAPREACNAWLQKRQRPEVFSDNMFCVGDETQRHSVCQGDSGSVYVVWDNHAHHWVATGIVSWGIGCGEGYDFYTKVLSYVDWIKGVMNGKN

SEQ ID NO: 5 - protein, 445 aa, Homo sapiens
MPGPRVWGKYLWRSPHSKGCPGAMWWLLLWGVLQACPTRGSVLLAQELPQQLTSPGYPEPYGKGQESSTDIKAPEGFAVRLVFQDFDLEPSQDCAGDSVTISFVGSDPSQFCGQQGSPLGRPPGQREFVSSGRSLRLTFRTQPSSENKTAHLHKGFLALYQTVGALTCATPGTWKDRQDGEEVLQCMPVCGRPVTPIAQNQTTLGSSRAKLGNFPWQAFTSIHGRGGGALLGDRWILTAAHTIYPKDSVSLRKNQSVNVFLGHTAIDEMLKLGNHPVHRVVVHPDYRQNESHNFSGDIALLELQHSIPLGPNVLPVCLPDNETLYRSGLLGYVSGFGMEMGWLTTELKYSRLPVAPREACNAWLQKRQRPEVFSDNMFCVGDETQRHSVCQGDSGSVYVVWDNHAHHWVATGIVSWGIGCGEGYDFYTKVLSYVDWIKGVMNGKN

SEQ ID NO: 6 - protein, 314 aa, Homo sapiens
MPGPRVWGKYLWRSPHSKGCPGAMWWLLLWGVLQACPTRGSVLLAQELPQQLTSPGYPEPYGKGQESSTDIKAPEGFAVRLVFQDFDLEPSQDCAGDSVTISFVGSDPSQFCGQQGSPLGRPPGQREFVSSGRSLRLTFRTQPSSENKTAHLHKGFLALYQTVAVNYSQPISEASRGSEAINAPGDNPAKVQNHCQEPYYQAAAAASTPSLFLCLSSFTPQGHSPVQPQGPGKTDRMGRRFFSVCLSADGQSPPLPRIRRPSVLPEPSWATSPGKPSPVSTAVGAGPCWGTDGSSLLPTPSTPRTVFLSGRTRV

SEQ ID NO: 7 - protein, 187 aa, Homo sapiens
MPGPRVWGKYLWRSPHSKGCPGAMWWLLLWGVLQACPTRGSVLLAQELPQQLTSPGYPEPYGKGQESSTDIKAPEGFAVRLVFQDFDLEPSQDCAGDSVTISFVGSDPSQFCGQQGSPLGRPPGQREFVSSGRSLRLTFRTQPSSENKTAHLHKGFLALYQTVGECPSWGCREGASVPSHDPGIFKP

SEQ ID NO: 8 - protein, 14 aa, Homo sapiens
VCGKPKNPANPVQR

SEQ ID NO: 9 - protein, 609 aa, Homo sapiens [sequence omitted]

SEQ ID NO: 10 - protein, 1121 aa, Homo sapiens
MSKLRMVLLEDSGSADFRRHFVNLSPFTITVVLLLSACFVTSSLGGTDKELRLVDGENKCSGRVEVKVQEEWGTVCNNGWSMEAVSVICNQLGCPTAIKAPGWANSSAGSGRIWMDHVSCRGNESALWDCKHDGWGKHSNCTHQQDAGVTCSDGSNLEMRLTRGGNMCSGRIEIKFQGRWGTVCDDNFNIDHASVICRQLECGSAVSFSGSSNFGEGSGPIWFDDLICNGNESALWNCKHQGWGKHNCDHAEDAGVICSKGADLSLRLVDGVTECSGRLEVRFQGEWGTICDDGWDSYDAAVACKQLGCPTAVTAIGRVNASKGFGHIWLDSVSCQGHEPAIWQCKHHEWGKHYCNHNEDAGVTCSDGSDLELRLRGGGSRCAGTVEVEIQRLLGKVCDRGWGLKEADVVCRQLGCGSALKTSYQVYSKIQATNTWLFLSSCNGNETSLWDCKNWQWGGLTCDHYEEAKITCSAHREPRLVGGDIPCSGRVEVKHGDTWGSICDSDFSLEAASVLCRELQCGTVVSILGGAHFGEGNGQIWAEEFQCEGHESHLSLCPVAPRPEGTCSHSRDVGVVCSRYTEIRLVNGKTPCEGRVELKTLGAWGSLCNSHWDIEDAHVLCQQLKCGVALSTPGGARFGKGNGQIWRHMFHCTGTEQHMGDCPVTALGASLCPSEQVASVICSGNQSQTLSSCNSSSLGPTRPTIPEESAVACIESGQLRLVNGGGRCAGRVEIYHEGSWGTICDDSWDLSDAHVVCRQLGCGEAINATGSAHFGEGTGPIWLDEMKCNGKESRIWQCHSHGWGQQNCRHKEDAGVICSEFMSLRLTSEASREACAGRLEVFYNGAWGTVGKSSMSETTVGVVCRQLGCADKGKINPASLDKAMSIPMWVDNVQCPKGPDTLWQCPSSPWEKRLASPSEETWITCDNKIRLQEGPTSCSGRVEIWHGGSWGTVCDDSWDLDDAQVVCQQLGCGPALKAFKEAEFGQGTGPIWLNEVKCKGNESSLWDCPARRWGHSECGHKEDAAVNCTDISVQKTPQKATTGRSSRQSSFIAVGILGVVLLAIFVALFFLTKKRRQRQRLAVSSRGENLVHQIQYREMNSCLNADDLDLMNSSGGHSEPH

SEQ ID NO: 11 - protein, 4544 aa, Homo sapiens
MLTPPLLLLLPLLSALVAAAIDAPKTCSPKQFACRDQITCISKGWRCDGERDCPDGSDEAPEICPQSKAQRCQPNEHNCLGTELCVPMSRLCNGVQDCMDGSDEGPHCRELQGNCSRLGCQHHCVPTLDGPTCYCNSSFQLQADGKTCKDFDECSVYGTCSQLCTNTDGSFICGCVEGYLLQPDNRSCKAKNEPVDRPPVLLIANSQNILATYLSGAQVSTITPTSTRQTTAMDFSYANETVCWVHVGDSAAQTQLKCARMPGLKGFVDEHTINISLSLHHVEQMAIDWLTGNFYFVDDIDDRIFVCNRNGDTCVTLLDLELYNPKGIALDPAMGKVFFTDYGQIPKVERCDMDGQNRTKLVDSKIVFPHGITLDLVSRLVYWADAYLDYIEVVDYEGKGRQTIIQGILIEHLYGLTVFENYLYATNSDNANAQQKTSVIRVNRFNSTEYQVVTRVDKGGALHIYHQRRQPRVRSHACENDQYGKPGGCSDICLLANSHKARTCRCRSGFSLGSDGKSCKKPEHELFLVYGKGRPGIIRGMDMGAKVPDEHMIPIENLMNPRALDFHAETGFIYFADTTSYLIGRQKIDGTERETILKDGIHNVEGVAVDWMGDNLYWTDDGPKKTISVARLEKAAQTRKTLIEGKMTHPRAIVVDPLNGWMYWTDWEEDPKDSRRGRLERAWMDGSHRDIFVTSKTVLWPNGLSLDIPAGRLYWVDAFYDRIETILLNGTDRKIVYEGPELNHAFGLCHHGNYLFWTEYRSGSVYRLERGVGGAPPTVTLLRSERPPIFEIRMYDAQQQQVGTNKCRVNNGGCSSLCLATPGSRQCACAEDQVLDADGVTCLANPSYVPPPQCQPGEFACANSRCIQERWKCDGDNDCLDNSDEAPALCHQHTCPSDRFKCENNRCIPNRWLCDGDNDCGNSEDESNATCSARTCPPNQFSCASGRCIPISWTCDLDDDCGDRSDESASCAYPTCFPLTQFTCNNGRCININWRCDNDNDCGDNSDEAGCSHSCSSTQFKCNSGRCIPEHWTCDGDNDCGDYSDETHANCTNQATRPPGGCHTDEFQCRLDGLCIPLRWRCDGDTDCMDSSDEKSCEGVTHVCDPSVKFGCKDSARCISKAWVCDGDNDCEDNSDEENCESLACRPPSHPCANNTSVCLPPDKLCDGNDDCGDGSDEGELCDQCSLNNGGCSHNCSVAPGEGIVCSCPLGMELGPDNHTCQIQSYCAKHLKCSQKCDQNKFSVKCSCYEGWVLEPDGESCRSLDPFKPFIIFSNRHEIRRIDLHKGDYSVLVPGLRNTIALDFHLSQSALYWTDVVEDKIYRGKLLDNGALTSFEVVIQYGLATPEGLAVDWIAGNIYWVESNLDQIEVAKLDGTLRTTLLAGDIEHPRAIALDPRDGILFWTDWDASLPRIEAASMSGAGRRTVHRETGSGGWPNGLTVDYLEKRILWIDARSDAIYSARYDGSGHMEVLRGHEFLSHPFAVTLYGGEVYWTDWRTNTLAKANKWTGHNVTVVQRTNTQPFDLQVYHPSRQPMAPNPCEANGGQGPCSHLCLINYNRTVSCACPHLMKLHKDNTTCYEFKKFLLYARQMEIRGVDLDAPYYNYIISFTVPDIDNVTVLDYDAREQRVYWSDVRTQAIKRAFINGTGVETVVSADLPNAHGLAVDWVSRNLFWTSYDTNKKQINVARLDGSFKNAVVQGLEQPHGLVVHPLRGKLYWTDGDNISMANMDGSNRTLLFSGQKGPVGLAIDFPESKLYWISSGNHTINRCNLDGSGLEVIDAMRSQLGKATALAIMGDKLWWADQVSEKMGTCSKADGSGSVVLRNSTTLVMHMKVYDESIQLDHKGTNPCSVNNGDCSQLCLPTSETTRSCMCTAGYSLRSGQQACEGVGSFLLYSVHEGIRGIPLDPNDKSDALVPVSGTSLAVGIDFHAENDTIYWVDMGLSTISRAKRDQTWREDVVTNGIGRVEGIAVDWIAGNIYWTDQGFDVIEVARLNGSFRYVVISQGLDKPRAITVHPEKGYLFWTEWGQYPRIERSRLDGTERVVLVNVSISWPNGISVDYQDGKLYWCDARTDKIERIDLETGENREVVLSSNNMDMFSVSVFEDFIYWSDRTHANGSIKRGSKDNATDSVPLRTGIGVQLKDIKVFNRDRQKGTNVCAVANGGCQQLCLYRGRGQRACACAHGMLAEDGASCREYAGYLLYSERTILKSIHLSDERNLNAPVQPFEDPEHMKNVIALAFDYRAGTSPGTPNRIFFSDIHFGNIQQINDDGSRRITIVENVGSVEGLAYHRGWDTLYWTSYTTSTITRHTVDQTRPGAFERETVITMSGDDHPRAFVLDECQNLMFWTNWNEQHPSIMRAALSGANVLTLIEKDIRTPNGLAIDHRAEKLYFSDATLDKIERCEYDGSHRYVILKSEPVHPFGLAVYGEHIFWTDWVRRAVQRANKHVGSNMKLLRVDIPQQPMGIIAVANDTNSCELSPCRINNGGCQDLCLLTHQGHVNCSCRGGRILQDDLTCRAVNSSCRAQDEFECANGECINFSLTCDGVPHCKDKSDEKPSYCNSRRCKKTFRQCSNGRCVSNMLWCNGADDCGDGSDEIPCNKTACGVGEFRCRDGTCIGNSSRCNQFVDCEDASDEMNCSATDCSSYFRLGVKGVLFQPCERTSLCYAPSWVCDGANDCGDYSDERDCPGVKRPRCPLNYFACPSGRCIPMSWTCDKEDDCEHGEDETHCNKFCSEAQFECQNHRCISKQWLCDGSDDCGDGSDEAAHCEGKTCGPSSFSCPGTHVCVPERWLCDGDKDCADGADESIAAGCLYNSTCDDREFMCQNRQCIPKHFVCDHDRDCADGSDESPECEYPTCGPSEFRCANGRCLSSRQWECDGENDCHDQSDEAPKNPHCTSQEHKCNASSQFLCSSGRCVAEALLCNGQDDCGDSSDERGCHINECLSRKLSGCSQDCEDLKIGFKCRCRPGFRLKDDGRTCADVDECSTTFPCSQRCINTHGSYKCLCVEGYAPRGGDPHSCKAVTDEEPFLIFANRYYLRKLNLDGSNYTLLKQGLNNAVALDFDYREQMIYWTDVTTQGSMIRRMHLNGSNVQVLHRTGLSNPDGLAVDWVGGNLYWCDKGRDTIEVSKLNGAYRTVLVSSGLREPRALVVDVQNGYLYWTDWGDHSLIGRIGMDGSSRSVIVDTKITWPNGLTLDYVTERIYWADAREDYIEFASLDGSNRHVVLSQDIPHIFALTLFEDYVYWTDWETKSINRAHKTTGTNKTLLISTLHRPMDLHVFHALRQPDVPNHPCKVNNGGCSNLCLLSPGGGHKCACPTNFYLGSDGRTCVSNCTASQFVCKNDKCIPFWWKCDTEDDCGDHSDEPPDCPEFKCRPGQFQCSTGICTNPAFICDGDNDCQDNSDEANCDIHVCLPSQFKCTNTNRCIPGIFRCNGQDNCGDGEDERDCPEVTCAPNQFQCSITKRCIPRVWVCDRDNDCVDGSDEPANCTQMTCGVDEFRCKDSGRCIPARWKCDGEDDCGDGSDEPKEECDERTCEPYQFRCKNNRCVPGRWQCDYDNDCGDNSDEESCTPRPCSESEFSCANGRCIAGRWKCDGDHDCADGSDEKDCTPRCDMDQFQCKSGHCIPLRWRCDADADCMDGSDEEACGTGVRTCPLDEFQCNNTLCKPLAWKCDGEDDCGDNSDENPEECARFVCPPNRPFRCKNDRVCLWIGRQCDGTDNCGDGTDEEDCEPPTAHTTHCKDKKEFLCRNQRCLSSSLRCNMFDDCGDGSDEEDCSIDPKLTSCATNASICGDEARCVRTEKAAYCACRSGFHTVPGQPGCQDINECLRFGTCSQLCNNTKGGHLCSCARNFMKTHNTCKAEGSEYQVLYIADDNEIRSLFPGHPHSAYEQAFQGDESVRIDAMDVHVKAGRVYWTNWHTGTISYRSLPPAAPPTTSNRHRRQIDRGVTHLNISGLKMPRGIAIDWVAGNVYWTDSGRDVIEVAQMKGENRKTLISGMIDEPHAIVVDPLRGTMYWSDWGNHPKIETAAMDGTLRETLVQDNIQWPTGLAVDYHNERLYWADAKLSVIGSIRLNGTDPIVAADSKRGLSHPFSIDVFEDYIYGVTYINNRVFKIHKFGHSPLVNLTGGLSHASDVVLYHQHKQPEVTNPCDRKKCEWLCLLSPSGPVCTCPNGKRLDNGTCVPVPSPTPPPDAPRPGTCNLQCFNGGSCFLNARRQPKCRCQPRYTGDKCELDQCWEHCRNGGTCAASPSGMPTCRCPTGFTGPKCTQQVCAGYCANNSTCTVNQGNQPQCRCLPGFLGDRCQYRQCSGYCENFGTCQMAADGSRQCRCTAYFEGSRCEVNKCSRCLEGACVVNKQSGDVTCNCTDGRVAPSCLTCVGHCSNGGSCTMNSKMMPECQCPPHMTGPRCEEHVFSQQQPGHIASILIPLLLLLLLVLVAGVVFWYKRRVQGAKGFQHQRMTNGAMNVEIGNPTYKMYEGGEPDDVGGLLDADFALDPDKPTNFTNPVYATLYMGGHGSRHSLASTDEKRELLGRGPEDEIGDPLA

SEQ ID NO: 12 - protein, 462 aa, Homo sapiens
MARVLGAPVALGLWSLCWSLAIATPLPPTSAHGNVAEGETKPDPDVTERCSDGWSFDATTLDDNGTMLFFKGEFVWKSHKWDRELISERWKNFPSPVDAAFRQGHNSVFLIKGDKVWVYPPEKKEKGYPKLLQDEFPGIPSPLDAAVECHRGECQAEGVLFFQGDREWFWDLATGTMKERSWPAVGNCSSALRWLGRYYCFQGNQFLRFDPVRGEVPPRYPRDVRDYFMPCPGRGHGHRNGTGHGNSTHHGPEYMRCSPHLVLSALTSDNHGATYAFSGTHYWRLDTSRDGWHSWPIAHQWPQGPSAVDAAFSWEEKLYLVQGTQVYVFLTKGGYTLVSGYPKRLEKEVGTPHGIILDSVDAAFICPGSSRLHIMAGRRLWWLDLKSGAQATWTELPWPHEKVDGALCMEKSLGPNSCSANGPGLYLIHGPNLYCYSDVEKLNAAKALPQPQNVTSLLGCTH

SEQ ID NO: 13 - protein, 357 aa, Homo sapiens
MAPRRVRSFLRGLPALLLLLLFLGPWPAASHGGKYSREKNQPKPSPKRESGEEFRMEKLNQLWEKAQRLHLPPVRLAELHADLKIQERDELAWKKLKLDGLDEDGEKEARLIRNLNVILAKYGLDGKKDARQVTSNSLSGTQEDGLDDPRLEKLWHKAKTSGKFSGEELDKLWREFLHHKEKVHEYNVLLETLSRTEEIHENVISPSDLSDIKGSVLHSRHTELKEKLRSINQGLDRLRRVSHQGYSTEAEFEEPRVIDLWDLAQSANLTDKELEAFREELKHFEAKIEKHNHYQKQLEIAHEKLRHAESVGDGERVSRSREKHALLEGRTKELGYTVKKHLQDLSGRISRARHNEL

SEQ ID NO: 14 - protein, 51 aa, Homo sapiens
VDSGNDVTDIADDGCPKPPEIAHGYVEHSVRYQCKNYYKLRTEGDGVYTLN

SEQ ID NO: 15 - protein, 229 aa, Homo sapiens
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 16 - protein, 217 aa, Mus musculus
APNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 17 - protein, 33 aa, Homo sapiens
NEKQWINKAVGDKLPECEAVCGKPKNPANPVQR

SEQ ID NO: 18 - protein, 8 aa, Artificial sequence
  Histidine tag
HHHHHHHH

SEQ ID NO: 19 - protein, 26 aa, Artificial sequence
  13xGly-Ser linker
GSGSGSGSGSGSGSGSGSGSGSGSGS

SEQ ID NO: 20 - protein, 84 aa, Homo sapiens
VDSGNDVTDIADDGCPKPPEIAHGYVEHSVRYQCKNYYKLRTEGDGVYTLNNEKQWINKAVGDKLPECEAVCGKPKNPANPVQR

SEQ ID NO: 21 - protein, 143 aa, Homo sapiens
VDSGNDVTDIADDGCPKPPEIAHGYVEHSVRYQCKNYYKLRTEGDGVYTLNDKKQWINKAVGDKLPECEADDGCPKPPEIAHGYVEHSVRYQCKNYYKLRTEGDGVYTLNNEKQWINKAVGDKLPECEAVCGKPKNPANPVQR